(19) United States
(12) Patent Application Publication
(10) Pub. No.: US 2025/0177974 A1
(43) Pub. Date: Jun. 5, 2025

(54) ARTICLES AND METHODS FOR CELL TRANSPORT

(71) Applicant: Trustees of Tufts College, Medford, MA (US)

(72) Inventors: Charles R. Mace, Winchester, MA (US); Lara P. Murray, Somerville, MA (US)

(73) Assignee: Trustees of Tufts College, Medford, MA (US)

(21) Appl. No.: 18/842,268

(22) PCT Filed: Mar. 2, 2023
(86) PCT No.: PCT/US2023/014369

§ 371 Date: Aug. 28, 2024

Related U.S. Application Data
(60) Provisional application No. 63/316,348, filed on Mar. 3, 2022.

(51) Int. Cl. B01L 3/00

(52) U.S. Cl. CPC B01L 3/502715; B01L 2300/0681; B01L 2300/069; B01L 2300/0861

(57) ABSTRACT

Aspects of the disclosure are directed toward fluidic devices that allow lateral and/or vertical transport of a fluid comprising cells, and possibly at least a portion of the cells disposed in the fluid, through one or more layers therein. For example, some fluidic devices comprise a layer comprising a channel that allows for lateral transport of a fluid comprising cells and/or cells disposed in such a fluid. As another example, some fluidic devices comprise a layer comprising a vertical transport region that allows for vertical transport of a fluid comprising cells and/or cells disposed in such a fluid. The fluidic devices described herein may also comprise one or more layers that filter cells and/or do not allow for transport of cells therethrough. The use of such layers in combination with layers allowing for lateral and/or vertical cell transport may advantageously allow cells to be transported through one or more portions of a device and retained at another portion of the device.

### RELATED APPLICATIONS

This application claims priority under 35 U.S.C. § 119 (e) to U.S. Provisional Application No. 63/316,348, filed Mar. 3, 2022, and entitled “Articles and Methods for Cell Transport,” which is incorporated herein by reference in its entirety.

### FIELD

Articles and methods related to cell transport are generally provided.

### BACKGROUND

Fluidic devices are sometimes employed to transport and/or analyze biological fluids. However, many fluidic devices are incapable of transporting cells or transport cells in a manner that has one or more drawbacks.

Accordingly, improved devices and methods are needed.

### SUMMARY

Articles and methods for cell transport are generally provided.

In some embodiments, the disclosure describes a fluidic device, comprising a first layer comprising a porous, absorbent material having a median pore size of greater than or equal to 15 microns and a mode pore size of greater than or equal to 15 microns, wherein the first layer comprises a channel, a first sample reception region, and a second sample reception region, and wherein the channel places the first sample reception region in fluidic communication with the second sample reception region; and a second layer wherein the second layer comprises a vertical transport region in fluidic communication with the first sample reception region and/or a sample collection region in fluidic communication with the second sample reception region.

In some embodiments, the disclosure describes a fluidic device, comprising a first layer comprising a first porous, absorbent material, wherein the first layer comprises a channel, a first sample reception region, and a second sample reception region, and wherein the channel places the first sample reception region in fluidic communication with the second sample reception region; and a second layer comprising a second porous, absorbent material having a median pore size of greater than or equal to 15 microns and a mode pore size of greater than or equal to 15 microns, wherein the second layer comprises a vertical transport region in fluidic communication with the first sample reception region.

In some embodiments, the disclosure describes a method, comprising laterally transporting a fluid comprising a plurality of cells through a channel, wherein the channel is positioned in a first layer comprising a porous, absorbent material having a median pore size of greater than or equal to 15 microns and a mode pore size of greater than or equal to 15 microns, the first layer further comprises a first sample reception region and a second sample reception region, the channel places the first sample reception region in fluidic communication with the second sample reception region, the cells are transported from the first sample reception region to the second sample reception region through the channel, the first layer is positioned in a fluidic device further comprising a second layer, and the second layer comprises a vertical transport region in fluidic communication with the first sample reception region and/or a sample collection region in fluidic communication with the second sample reception region.

In some embodiments, the disclosure describes a method, comprising vertically transporting a fluid comprising a plurality of cells through a vertical transport region, wherein the vertical transport region is in fluidic communication with a first sample reception region, the first sample reception region is positioned in a first layer of a device, the first layer of the device comprises a porous, absorbent material having a median pore size of greater than or equal to 15 microns and a mode pore size of greater than or equal to 15 microns, the first layer further comprises a channel and a second sample reception region, the channel places the first sample reception region in fluidic communication with the second sample reception region, the cells are transported to the first sample reception region to the second sample reception region through the vertical transport region, and the vertical transport region is positioned in a second layer of the fluidic device.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows a schematic depiction of a device comprising a lateral transport layer including a channel, a first sample reception region, and a second sample reception region, according to some embodiments;

FIG. 2 shows a schematic depiction of a device comprising a vertical transport layer disposed on a lateral transport layer, according to some embodiments;

FIG. 3 shows a schematic depiction of a device comprising a lateral transport layer disposed on a sample collection layer, according to some embodiments;

FIG. 4 shows a schematic depiction of a device comprising a vertical transport layer, a lateral transport layer, and a sample collection layer, according to some embodiments;

FIG. 5 shows a schematic depiction of a device comprising a lateral transport layer, a sample collection layer, and a wash layer, according to some embodiments;

FIG. 6 shows a schematic depiction of a device comprising a lateral transport layer, a sample collection layer, a wash layer, and a blotting layer, according to some embodiments;

FIG. 7 shows a schematic depiction of a device comprising a filter disposed between a vertical transport layer and a lateral transport layer, according to some embodiments;

FIG. 8 shows a schematic depiction of a device comprising a filter disposed between a lateral transport layer and a sample collection layer, according to some embodiments;

FIGS. 9-11 show schematic depictions devices comprising splitting layers disposed on vertical transport layers, according to some embodiments;

FIGS. 12A-12D show fluorescent images of various porous materials in which channels have been formed: Kimwipes (FIG. 12A), Clever coffee filters (FIG. 12B), Technicloth synthetic wipers (FIG. 12C), and TX1109 (FIG. 12D), according to some embodiments;

FIGS. 13A-13I show fluorescent images of various porous materials in which channels have been formed: Kimwipes (FIG. 13A), Clever coffee filters (FIG. 13B), Technicloth synthetic wipers (FIG. 13C), TX1109 (FIG. 13D), Professional WypAll X60 Wipers (FIG. 13E), WypAll X70 wipers (FIG. 13F), DURX 670 non-woven polyester/cellulose wipers (FIG. 13G), TX612 Technicloth nonwoven wipers (FIG. 13H), and Ahlstrom 55 filter paper (FIG. 13I) according to some embodiments;

FIG. 14A shows a schematic depiction of a device employed to perform affinity-based labeling and enumeration of cells from a fluid sample, according to some embodiments;

FIG. 14B shows a legend indicating treatments applied to the vertical transport layer depicted in FIG. 14A, according to some embodiments;

FIG. 14C shows a schematic of a detection strategy that used to detect the cells, according to some embodiments;

FIGS. 15A-15B show flow cytometry results for immunophenotyping of Jurkat D1.1 (FIG. 15A) and MAVER-1 cells (FIG. 15B), according to some embodiments;

FIG. 15C depicts the legend for the charts shown in FIGS. 15A and B, according to some embodiments;

FIGS. 16A-16B illustrates the thresholding settings using in ImageJ (FIG. 16A) and the corresponding masks generated (FIG. 16B) for image analysis of scanned images of the sample collection region, according to some embodiments;

FIGS. 17A-17D show representative scans of stained sample collection regions following affinity-based separation of Jurkat D1.1 (CD3+/CD19−) cells (FIG. 17A) and MAVER-1 (CD3−/CD19+) cells (FIG. 17C) and the accompanying calibration curves generated for CD3+/CD19− cells (FIG. 17B) and CD3−/CD19+ cells (FIG. 17D), according to some embodiments;

FIGS. 18A-18B show quantitative analysis of the CD3+/CD19− cells shown in FIG. 17B (FIG. 18A) and FIG. 17D (FIG. 18B), according to some embodiments;

FIG. 19 shows the pore-size distribution of Clever coffee filters, according to some embodiments;

FIGS. 20A and 20C show the ability of devices with anti-CD3-HRP embedded in the vertical transport region to specifically label Jurkat D1.1 (CD3+/CD19−) cells (FIG. 20A) but not MAVER-1 (CD3−/CD19+) cells (FIG. 20C), according to some embodiments;

FIGS. 20B and 20D show the ability of devices with anti-CD19-HRP embedded in the vertical transport region to specifically label MAVER-1 (CD3+/CD19−) cells (FIG. 20 D), but not Jurkat D1.1 (CD3+/CD19−) cells (FIG. 20B), according to some embodiments;

FIG. 21 shows a schematic of a device with PCTE filters for size exclusion filtration, according to some embodiments;

FIGS. 22A-22D show optical and fluorescent micrographs of anti-CD14-PE labeled monocytes being captured within a 10 micron PCTE filter (FIG. 22A-B);

FIGS. 23A-23D show optical and fluorescent micrographs of anti-CD14-PE labeled monocytes being captured within a 12 micron PCTE filter (FIG. 23A-B);

FIGS. 24A-24D show optical and fluorescent micrographs of anti-CD14-PE labeled monocytes being captured within a 14 micron PCTE filter (FIG. 24A-B);

FIGS. 25A-25D show optical and fluorescent micrographs of a sample collection layer following passage of a solution containing a mixture of anti-CD4-PE labeled T-cells and monocytes through a 10 micron PCTE membrane disposed thereon, according to some embodiments;

FIGS. 26A-26D show optical and fluorescent micrographs of a sample collection layer following passage of a solution containing a mixture of anti-CD4-PE labeled T-cells and monocytes through a 12 micron PCTE membrane disposed thereon, according to some embodiments;

FIGS. 27A-27D show optical and fluorescent micrographs of a sample collection layer following passage of a solution containing a mixture of anti-CD4-PE labeled T-cells and monocytes through a 14 micron PCTE membrane disposed thereon, according to some embodiments;

FIGS. 28A-28D show optical and fluorescent micrographs of a sample collection layer following passage of a solution containing PBMCs labeled with either PE-anti-CD4 or PE-anti-CD14 through a device lacking a PCTE membranes, according to some embodiments;

FIGS. 29-31 show schematic depictions of devices comprising splitting layers, according to some embodiments;

FIGS. 32A-32D show fluorescent micrographs of the sample collection region following passage of either anti-CD4 labeled CEMs or unlabeled CEMs through a device shown in FIG. 31, according to some embodiments;

FIG. 33 shows the quantification of the fluorescent intensity from the images shown in FIG. 32A-D, according to some embodiments;

FIG. 34 shows a schematic depicting the use of a protein-A-cellulose binding domain bound to a cellulose-based material to capture a cell of interest, according to some embodiments;

FIGS. 35A-35H show fluorescent micrographs following addition of CEM (CD4+/CD20−) or MAVER-1 (CD4−/CD20+) cells to a device comprising a vertical transport layer comprising a polyester material functionalized with either anti-CD4 or anti-CD20, according to some embodiments;

FIG. 36 is a schematic illustration of a multiplexed device comprising a 2-channel splitting layer, a lateral transport layer, and a sample collection layer, in some embodiments;

FIG. 37 includes fluorescent micrographs of the splitting layer, the lateral transport layer, and the sample collection layer of the device shown in FIG. 37 following addition of fluorescently-labeled MAVER-1 cells;

FIG. 38A is a schematic illustration of a multiplexed device comprising a 2-channel splitting layer, a sample collection layer, and a blotting layer, according to some embodiments;

FIG. 38B includes fluorescent micrographs of the 2-channel splitting layer shown in FIG. 38A and the sample collection layer following addition of fluorescently-labeled MAVER-1 cells, according to some embodiments;

FIG. 39A is a schematic illustration of a multiplexed device comprising a 3-channel splitting layer, a sample collection layer, and a blotting layer, according to some embodiments;

FIG. 39B includes fluorescent micrographs of the 3-channel splitting layer and the sample collection layer shown in FIG. 39A following addition of fluorescently-labeled MAVER-1 cells, according to some embodiments;

FIG. 40A is a schematic illustration of a multiplexed device comprising a 4-channel splitting layer, a sample collection layer, and a blotting layer, according to some embodiments;

FIG. 40B includes fluorescent micrographs of the 4-channel splitting layer and the sample collection layer shown in FIG. 40B following addition of fluorescently-labeled MAVER-1 cells, according to some embodiments;

FIGS. 41A-41E show pore size distributions for a variety of porous materials, in accordance with some embodiments;

FIG. 42 compares the size of MAVER-1 cells to pores of various sizes, in accordance with some embodiments;

FIG. 43 shows the percent area covered by cells as a function of the volume of cells added to a fluidic device, in accordance with some embodiments;

FIG. 44 shows CD4 calibration curves, in accordance with some embodiments;

FIG. 45 shows representative scans of a sample collection layer of a fluidic device, in accordance with some embodiments;

FIG. 46 demonstrates an assay that provides a semi-quantitative readout, in accordance with some embodiments;

FIGS. 47A-47D show exemplary calibration curves and associated optical micrographs, in accordance with some embodiments;

FIGS. 48A-48C show exemplary calibration curves and various features of a fluidic device for detecting Jurkat D1.1 and MAVER-1 cells, in accordance with some embodiments;

FIGS. 49A and 49B show exemplary calibration curves and associated optical micrographs, in accordance with some embodiments; and

FIGS. 50A and 50B show fluorescence micrographs, in accordance with some embodiments.

### DETAILED DESCRIPTION

Aspects of the disclosure are directed toward fluidic devices that allow lateral and/or vertical transport of a fluid comprising cells, and possibly at least a portion of the cells disposed in the fluid, through one or more layers therein. For example, some fluidic devices comprise a layer comprising a channel that allows for lateral transport of a fluid comprising cells and/or cells disposed in such a fluid. As another example, some fluidic devices comprise a layer comprising a vertical transport region that allows for vertical transport of a fluid comprising cells and/or cells disposed in such a fluid. The fluidic devices described herein may also comprise one or more layers that filter cells and/or do not allow for transport of cells therethrough. The use of such layers in combination with layers allowing for lateral and/or vertical cell transport may advantageously allow cells to be transported through one or more portions of a device and retained at another portion of the device.

As such, the fluidic devices described herein may be advantageous for one or more reasons. For example, some fluidic devices may be capable of labeling a specific population of cells in a fluid (e.g., during immunophenotyping) at one or more locations in a fluidic device, such as one or more locations to which a fluid comprising cells can laterally and/or vertically flow. This may allow for cell labeling in a manner that is particularly facile. Cell labeling may be achieved, for example, by transporting a fluid comprising cells through a porous, absorbent material onto which an affinity agent is adsorbed. Upon contact of the fluid with the affinity agent, the affinity agent may be solubilized. The solubilized affinity agent may mix with the cells and label the cells as they are transported through the porous, absorbent material. After cell labeling, the cells may undergo further lateral and/or vertical flow. For instance, they may be further transported to a portion of the fluidic device where they can be trapped.

In some instances, a fluidic device allows for lateral and/or vertical flow of a fluid across one or more distances that are particularly advantageous. As one example, in some fluidic devices, the distance between a location at which cells are exposed to an affinity agent and a location at which cells are trapped may be sufficiently long to allow for adequate labeling of the cells with the affinity agent prior to cell trapping. Such adequate labeling may enhance the efficiency with which cells are trapped and/or the signal generated by trapped cells.

As another example of an advantage that may be present in some of the fluidic devices described herein, some fluidic devices may be capable of running multiple analyses on a single fluid introduced to the device (e.g., multiplexing). This can be achieved, for example, by incorporating a lateral transport layer having multiple channels emanating from a central sample addition region, such that, upon addition of a fluid, each channel transports an aliquot of the sample laterally away from the sample addition region. Once the fluid has been split, different analyses may be performed on each of the aliquots, such as for example, different affinity-based labeling processes (e.g., identification of two or more different cell populations within a fluid sample). Beneficially, performing different analyses on different aliquots of a common fluid may allow for multiple features of a single fluid to be determined in a single fluidic device and/or may reduce the labor and/or waste associated with dividing a fluid into multiple aliquots prior to analysis. For example, multiplexing may reduce the sample volume needed to perform multiple analyses, relative to the sample volume required to run each analysis separately. This may be beneficial in devices designed for point of care diagnostics, as it may allow multiple tests to be run from a single drop of blood (e.g., obtained from a fingerstick), which may desirably reduce the number of finger sticks that need to be performed.

Some fluidic devices described herein may advantageously be capable of performing complex sample processing in situ. For instance, some fluidic devices may be capable of performing multiple steps in sequence that result in the capturing of a relatively pure sample of labeled white blood cells from a fluid initially also comprising red blood cells (e.g., whole blood). Such devices may be advantageous for use with fluids comprising an appreciable number of red blood cells (e.g., whole blood, which is believed to include red blood cells in an amount that is 600 times higher than white blood cells). The multiple steps may comprise both trapping the red blood cells and labelling the white blood cells. In some fluidic devices, the red blood cells may be trapped prior to the labelling of the white blood cells. As red blood cells are larger than white blood cells, trapping the red blood cells prior to labelling the white blood cells may facilitate transport of the resultant fluid through portions of the fluidic device comprising pores large enough to allow for white blood cell transport therethrough but too small to allow for red blood cell transport therethrough.

Additionally, some fluidic devices described herein may comprise one or more features that yield further benefits. For example, some fluidic devices may be capable of being employed as point of care diagnostics in resource-limited regions. The devices described herein may be suitable for such applications because they may be capable of being employed without the use of equipment that such regions may lack, such as external pumps and complicated optical equipment.

Various designs for fluidic devices, and accompanying FIGs., are discussed below. It should be understood that embodiments may encompass fluidic devices having some or all of the features depicted in one or more of the FIGs. It is also possible for a fluidic device to comprise combinations of features depicted in two or more FIGs. and/or to comprise one or more features not depicted in any FIG. Similarly, it should be understood that the numbering of layers within the devices described herein is arbitrary. Any of the layers described below and elsewhere herein may be a first layer of a fluidic device described herein, a second layer of a fluidic device described herein, a third layer of a fluidic device described herein, a fourth layer of a fluidic device described herein, and/or a fifth layer of a fluidic device described herein.

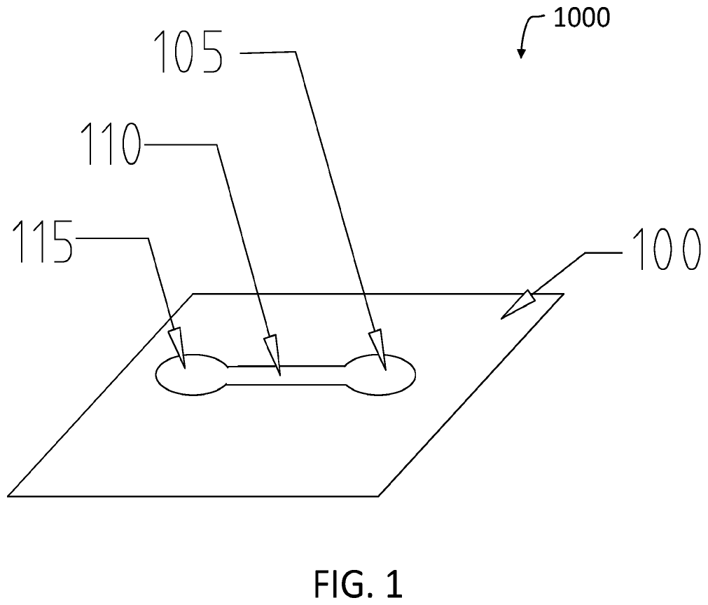

FIG. 1 shows one non-limiting embodiment of a fluidic device comprising a layer comprising a channel and two sample reception regions. In FIG. 1, a fluidic device 1000 comprises a lateral transport layer 100. The lateral transport layer may be referred to elsewhere herein as a first layer, a second layer, a third layer, a fourth layer, a fifth layer, and/or a sixth layer. The lateral transport layer 100 shown in FIG. 1 comprises a channel 110, a first sample reception region 115 and a second sample reception region 105. The channel 110 places the first sample reception region 115 in fluidic communication with the second sample reception region 105. In other words, the channel 110 allows fluid to flow from the first sample reception region 115 to the second sample reception region 105 (and vice versa). In some embodiments, the first sample reception region 115 is configured to receive a fluid comprising cells (e.g., from the environment external to the fluidic device). The sample received by the first sample reception region 115 may be transported to the second sample reception region 125. This may be accomplished by flowing from the first sample reception region, through the channel 110 (e.g., laterally and/or vertically), to the second sample reception region 105. In some embodiments, and as shown illustratively in FIG. 1, the channel 110 connects the first sample reception region 115 to the second sample reception region 105. As described above, this design may be advantageous for transporting a fluid comprising cells and/or cells disposed in such a fluid from the first sample reception region to the second sample reception region.

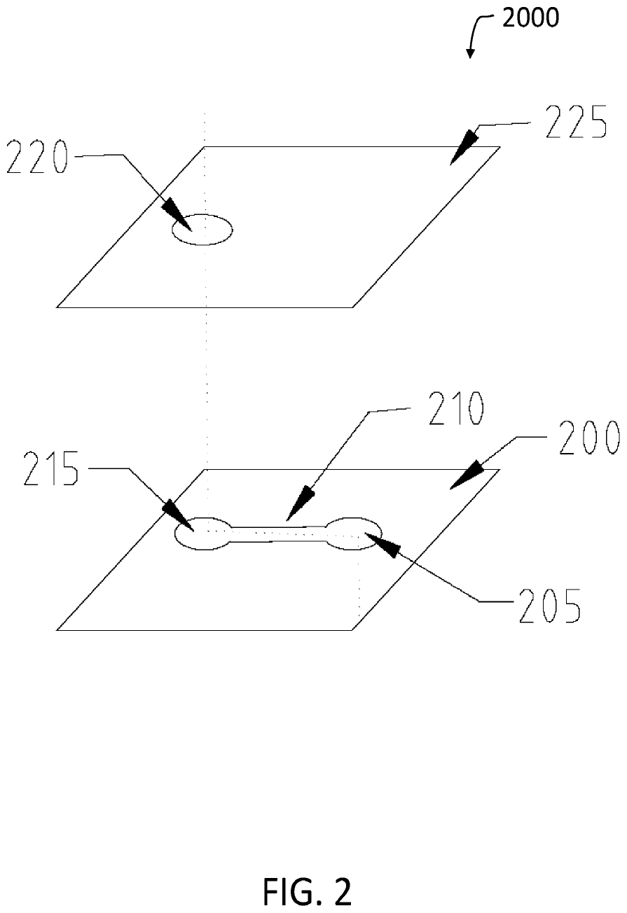

FIG. 2 shows one non-limiting embodiment of a fluidic device comprising two layers. In FIG. 2, a fluidic device 2000 comprises a lateral transport layer 200 and a vertical transport layer 225. The vertical transport layer may be referred to elsewhere herein as a first layer, a second layer, a third layer, a fourth layer, a fifth layer, and/or a sixth layer. The lateral transport layer 200 shown in FIG. 2 comprises a channel 210, a first sample reception region 215 and a second sample reception region 205. The channel 210 places the first sample reception region 215 in fluidic communication with the second sample reception region 205. The vertical transport layer 225 comprises a vertical transport region 220. In some embodiments, the vertical transport region 220 is in fluidic communication with the first sample reception region 215. For instance, the vertical transport region 220 may contact the first sample reception region 215. As another example, the vertical transport region 220 may be disposed above or below the first sample reception region 215 and at least partially laterally overlap the first sample reception region 215.

In some embodiments, a vertical transport region is configured to receive a fluid comprising cells (e.g., from the environment external to the fluidic device). Some methods may comprise adding a fluid comprising cells to the vertical transport region. The fluid comprising cells received by the vertical transport region may flow therethrough (e.g., laterally and/or vertically). In some embodiments, the fluid comprising cells may be transported (e.g., it may flow) from the vertical transport region 220 to the first sample reception region. The fluid comprising cells received by the first sample reception region may flow from the first sample reception region, through the channel (e.g., laterally and/or vertically), to the second sample reception region.

In some embodiments, an antibody is positioned in a vertical transport region. Vertical transport regions may comprise an affinity agent (e.g., an antibody) that is capable of being removed (e.g., solubilized) by a fluid comprising cells added thereto and/or may comprise an affinity agent that is incapable of being removed by a fluid comprising cells exposed thereto. Fluidic devices comprising an affinity agent disposed in this location that is capable of being removed may be useful, for example, for labeling cells (e.g., a subpopulation of cells) within a fluid with the affinity agent. In some embodiments, such a design may permit transport of labeled cells (and, possibly, cells not labeled by the affinity agent) to one or more further locations in the fluidic device (e.g., the first sample reception region, the second sample reception region). Fluidic devices comprising an affinity agent that is incapable of being removed by a fluid exposed thereto may bind to cells (e.g., a subpopulation of cells, such as a subpopulation of immune cells) therein. This may, for example, prevent a specific cell population (e.g., the subpopulation of bound cells) from flowing into the first sample reception region, such that the fluid transported to the second sample reception region lacks such cells and/or comprises such cells in a reduced amount. If the fluid comprises a different, unbound subpopulation of cells, it may become enriched in that particular cell subpopulation.

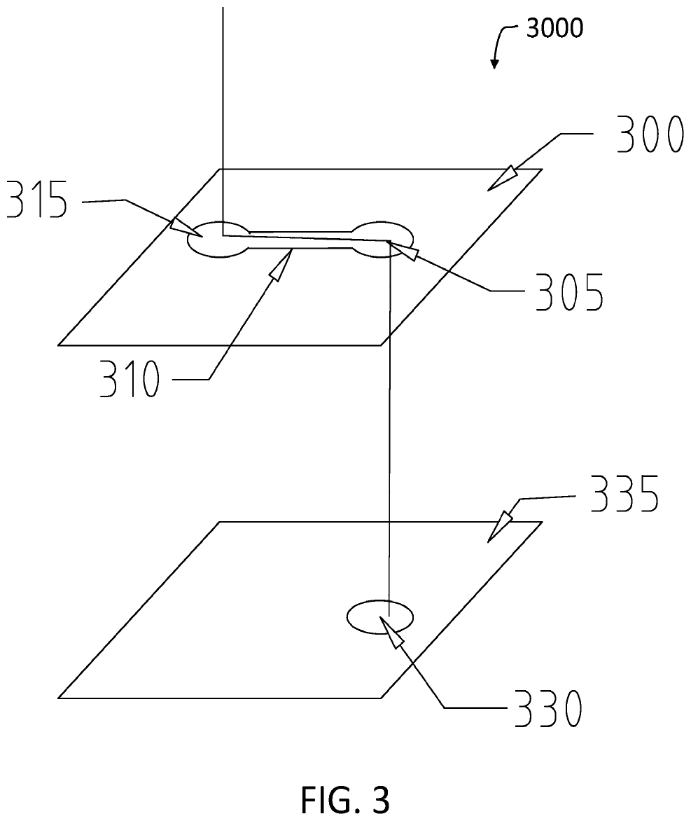

FIG. 3 shows another non-limiting embodiment of a fluidic device comprising two layers. In FIG. 3, a fluidic device 3000 comprises a lateral transport layer 300 and a sample collection layer 335. The sample collection layer may be referred to elsewhere herein as a first layer, a second layer, a third layer, a fourth layer, a fifth layer, and/or a sixth layer. The lateral transport layer 300 comprises a channel 310, a first sample reception region 315 and a second sample reception region 305. The channel 310 places the first sample reception region 315 in fluidic communication with the second sample reception region 305. The sample collection layer 335 comprises a sample collection region 330. In some embodiments, the sample collection region 330 is in fluidic communication with the second sample reception region 305. For instance, the sample collection region 330 may contact the second sample reception region 305. As another example, the sample collection region 330 may be disposed above or below the second sample reception region 305 and at least partially laterally overlap the second sample reception region 305.

In some embodiments, a fluidic device is designed such that a first sample reception region is configured to receive a fluid comprising cells (e.g., from the environment external to the fluidic device). The fluid comprising cells received by the first sample reception region may flow from the first sample reception region, through the channel (e.g., laterally and/or vertically), to the second sample reception region. The fluid comprising the cells may then be transported to the sample collection region. This may be accomplished by flow through the second sample reception region 305 (e.g., laterally and/or vertically) to the sample collection region. In some embodiments, the sample collection layer comprises pores that are small enough to trap, for example, cells within the fluid. Cells trapped in the sample collection layer may layer be recovered and/or analyzed. As such, in some embodiments, a sample collection layer is reversibly attached to a lateral transport layer, such that the sample collection layer can be removed from the lateral transport layer (e.g. by peeling), without the use of specialized tools, and/or without destroying the first layer.

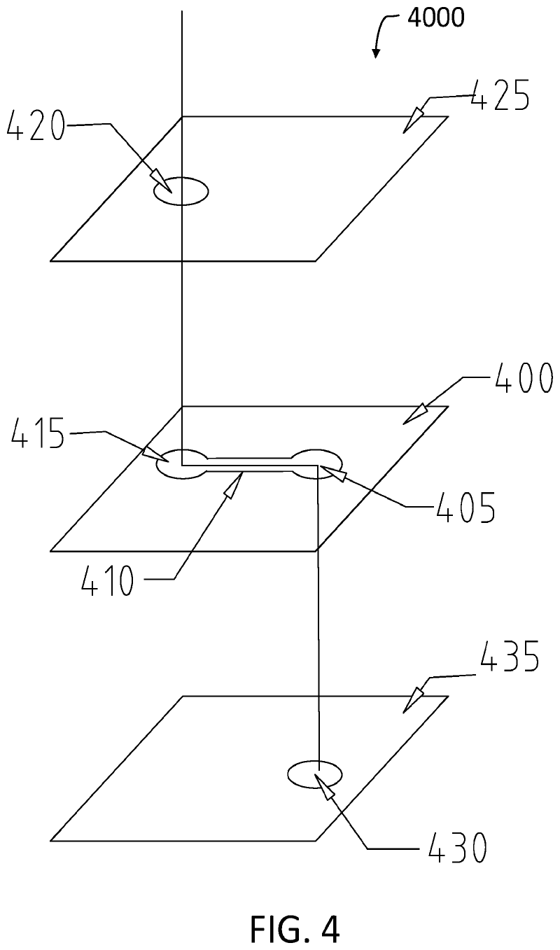

FIG. 4 shows one non-limiting embodiment of a fluidic device comprising three layers. In FIG. 4, a fluidic device 4000 comprises a vertical transport layer 425, a lateral transport layer 400, and a sample collection layer 435. In FIG. 4, the vertical transport layer and the sample collection layer are positioned on opposite sides of the lateral transport layer. Fluidic devices having the design shown in FIG. 4 may comprise a lateral transport layer as shown in any one of FIGS. 1-3, a vertical transport layer as shown in FIG. 2, and/or a sample collection layer as shown in FIG. 3.

In some embodiments, as described above, a fluidic device comprises a vertical transport region in which an affinity agent is positioned. As also described above, a fluid received by the vertical transport region may both solubilize the affinity agent and flow through the vertical transport region. The fluid in which the affinity agent is solubilized may then flow through and/or to other portions of the fluidic device, such as a lateral transport layer (e.g., through a first sample reception region therein, through a channel therein, through a second sample reception region therein) and/or a sample collection layer (e.g., to a sample collection region therein). During this flow, the affinity agent may mix with the fluid and/or label one or more cells in the fluid. Upon flow of the fluid to the sample collection layer, cells (e.g., cells labeled by the affinity agent, cells unlabeled by the affinity agent) may become trapped in the sample collection region. In some embodiments, a sample collection layer is treated with a detection reagent and used, for example, to quantify the concentration of labeled cells within the fluid sample. It is also possible for a sample collection layer to comprise a stored detection reagent.

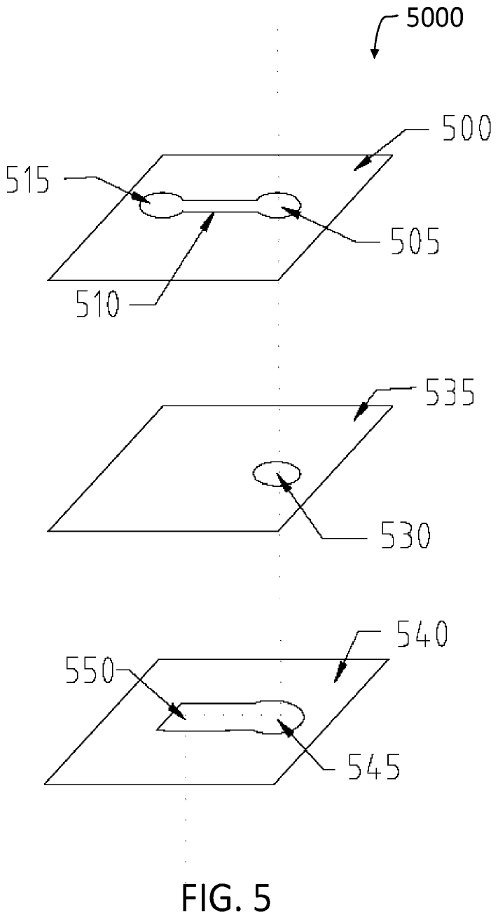

FIG. 5 shows another non-limiting embodiment of a fluidic device comprising three layers. In FIG. 5, a fluidic device 5000 comprises a lateral transport layer 500, a sample collection layer 535, and a wash layer 540. The wash layer may be referred to elsewhere herein as a first layer, a second layer, a third layer, a fourth layer, a fifth layer, and/or a sixth layer. Fluidic devices having the design shown in FIG. 5 may comprise a lateral transport layer as shown in any one of FIGS. 1-4, a vertical transport layer as shown in any one of FIGS. 2 and 4, and/or a sample collection layer as shown in any one of FIGS. 3-4.

In some embodiments, the wash layer shown in FIG. 5 comprises a porous material comprising a wash region 545 and a channel 550 in fluidic communication with the wash region 545. In some embodiments, the wash region is 545 is in fluidic communication with a sample collection region (e.g., the sample collection region 530 shown in FIG. 5). For instance, the wash region may contact the sample collection region. As another example, the wash region may be disposed below the sample collection region and at least partially laterally overlap the sample collection region.

In some embodiments, the device shown in FIG. 5 is configured to receive a fluid comprising cells (e.g., from the environment external to the fluidic device). The fluid comprising cells may flow through the fluidic device, and at least a portion of the fluid comprising cells (e.g., an aqueous fraction thereof) may flow to and/or through the wash region. In some embodiments, a fluid received by the fluidic device may be received by a vertical transport region, and sequentially flow through the vertical transport region, a first sample reception region, a channel, a second sample reception region. At this point, some or all of the cells in the fluid may be trapped within the sample collection region. One or more other portions of the fluid (e.g., an aqueous fraction thereof) may flow through the sample collection region and into the wash region. In this manner, an aqueous fraction of a fluid comprising cells may be transported to the wash region. In some embodiments, the aqueous fraction may be further transported to another portion of the wash layer (e.g., a channel in the wash layer).

Fluidic devices including a layer, like the wash layer, into which some, but not all, of a fluid may flow may be advantageous. Such fluidic devices may, for example, allow a portion of a fluid (e.g., an aqueous fraction thereof) to flow through and away from a region in which cells are trapped (e.g., a sample collection region). The flow of the portion of the fluid through and away from such regions may reduce background noise in a signal arising from the region in which the cells are trapped (and thereby may increase device sensitivity to such cells) by removing the other portion of the fluid therefrom. The portion of the fluid removed from the region in which the cells are trapped may comprise one or more components (e.g., unbound soluble antibodies) that would generate such background noise.

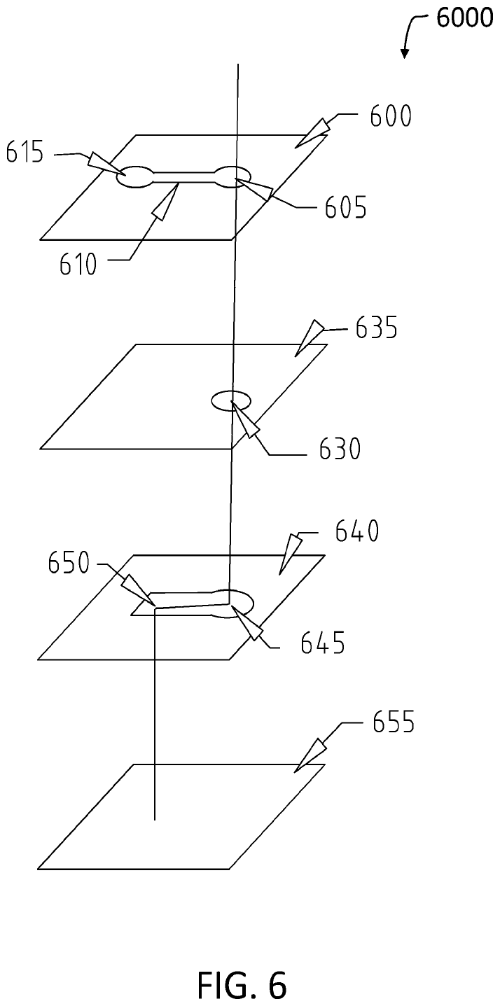

FIG. 6 shows non-limiting embodiment of a fluidic device comprising four layers. In FIG. 6, a fluidic device 6000 comprises a lateral transport layer 600, a sample collection layer 635, a wash layer 640, and a blotting layer 655. The blotting layer may be referred to elsewhere herein as a first layer, a second layer, a third layer, a fourth layer, a fifth layer, and/or a sixth layer. Fluidic devices having the design shown in FIG. 6 may comprise a lateral transport layer as shown in any one of FIGS. 1-5, a vertical transport layer as shown in any one of FIGS. 2 and 4-5, a sample collection layer as shown in any one of FIGS. 3-5, and/or a wash layer as shown in FIG. 5.

In some embodiments, the device shown in FIG. 6 is configured to receive a fluid comprising cells (e.g., from the environment external to the fluidic device). The fluid comprising cells may flow through the fluidic device, and at least a portion of the fluid comprising cells may flow to the blotting layer. In some embodiments, a fluid received by the fluidic device may be received by a vertical transport region, and sequentially flow through the vertical transport region, a first sample reception region, a channel, a second sample reception region. At this point, some or all of the cells in the fluid may be trapped within the sample collection region. One or more other portions of the fluid (e.g., an aqueous fraction thereof) may flow through the sample collection region and into the wash region. Some or all of these portions of the fluid may then flow through the wash region and into the blotting layer. Some or all of these portions of the fluid may then be absorbed by the blotting layer.

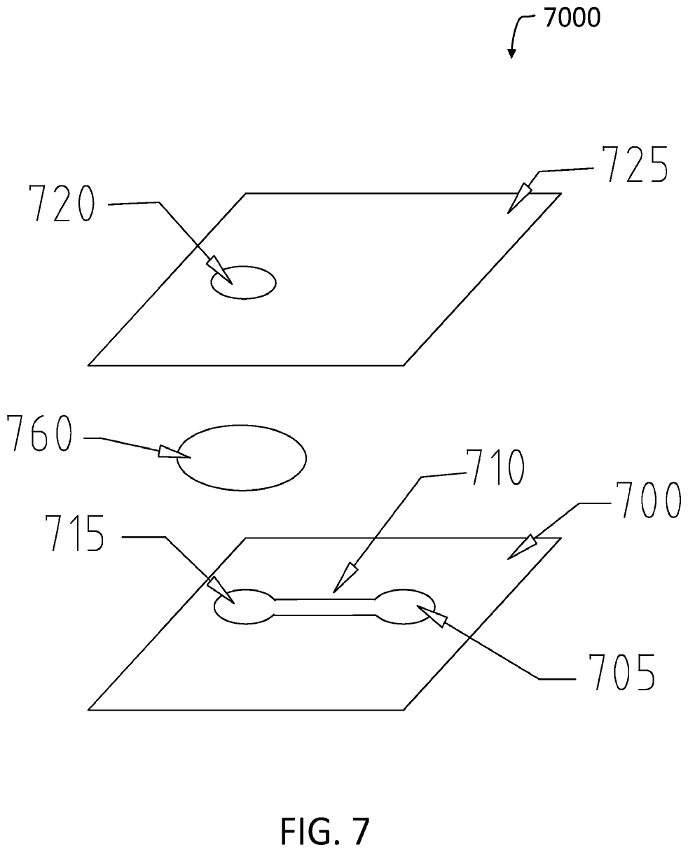

FIG. 7 shows one non-limiting embodiment of a fluidic device comprising a filter. In FIG. 7, a fluidic device 7000 comprises a vertical transport layer 725, a filter 760, and a lateral transport layer 700. Fluidic devices having the design shown in FIG. 7 may comprise a lateral transport layer as shown in any one of FIGS. 1-6 and/or a vertical transport layer as shown in any one of FIGS. 2 and 4-6. Such fluidic devices may also comprise one or more further layers described elsewhere herein but not depicted in FIG. 7 (e.g., a sample collection layer, a wash layer, a blotting layer). The filter 760 shown in FIG. 7 may comprise a porous material configured to exclude particulates larger than its median pore size.

In some embodiments, a fluidic device is configured to receive a fluid comprising a plurality of cells of different sizes. This fluid may be received in the vertical transport region 720. The fluid may then flow from the vertical transport region 720 into the filter 760. At least a portion of the fluid received by filter 760 may become trapped within the filter (e.g., the filter may trap cells having sizes larger than the median pore size of the filter). It is also possible for at least a portion of the fluid received by the filter 760 to flow to the first sample reception region 715 (e.g., cells having sizes smaller than the median pore size of the filter may pass through the filter, an aqueous fraction may pass through the filter). The fluid received by the first sample reception region may flow from the first sample reception region 715, through the channel 710, into the second sample reception region 705. Fluidic devices comprising a filter that traps at least a portion of a fluid to which it is exposed may be useful, for example, for separating a subpopulation of cells (e.g., monocytes) within a fluid sample (e.g., a whole blood sample).

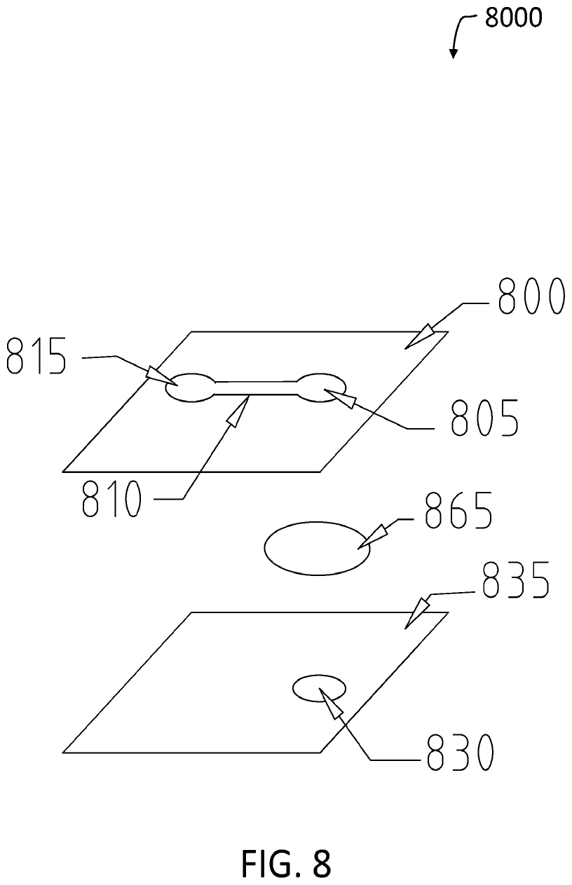

FIG. 8 shows another non-limiting embodiment of a fluidic device comprising a filter. In FIG. 8, a fluidic device 8000 comprises a lateral transport layer 800, a filter 865, and a sample collection layer 835. Fluidic devices having the design shown in FIG. 8 may comprise a lateral transport layer as shown in any one of FIGS. 1-7 and/or a sample collection layer as shown in any one of FIGS. 3-5 and 7. Such fluidic devices may also comprise one or more further layers described elsewhere herein but not depicted in FIG. 8 (e.g., a vertical transport layer, a wash layer, a blotting layer). The filter 865 shown in FIG. 8 comprises a porous material configured to exclude particulates larger than its median pore size.

As described above, in some embodiments, a fluidic device is configured to receive a fluid comprising a plurality of cells of different sizes. This fluid may be received by the first sample reception region 815. The fluid may then flow through the channel 810 until it reaches the second sample reception region 805. The sample received by the second sample reception region 805 may then flow vertically into the filter 865. At least a portion of the fluid received by filter 865 may become trapped within the filter (e.g., the filter may trap cells having sizes larger than the median pore size of the filter). It is also possible for at least a portion of the fluid received by the filter 865 to flow to the sample collection region (e.g., cells having sizes smaller than the median pore size of the filter may pass through the filter, an aqueous fraction may pass through the filter). Fluidic devices comprising a filter positioned between the second sample reception region and the sample collection region may be useful, for example, for separating two types of cells initially present in a common fluid. For instance, such a fluidic device may be capable of trapping monocytes on the and lymphocytes on the sample collection region.

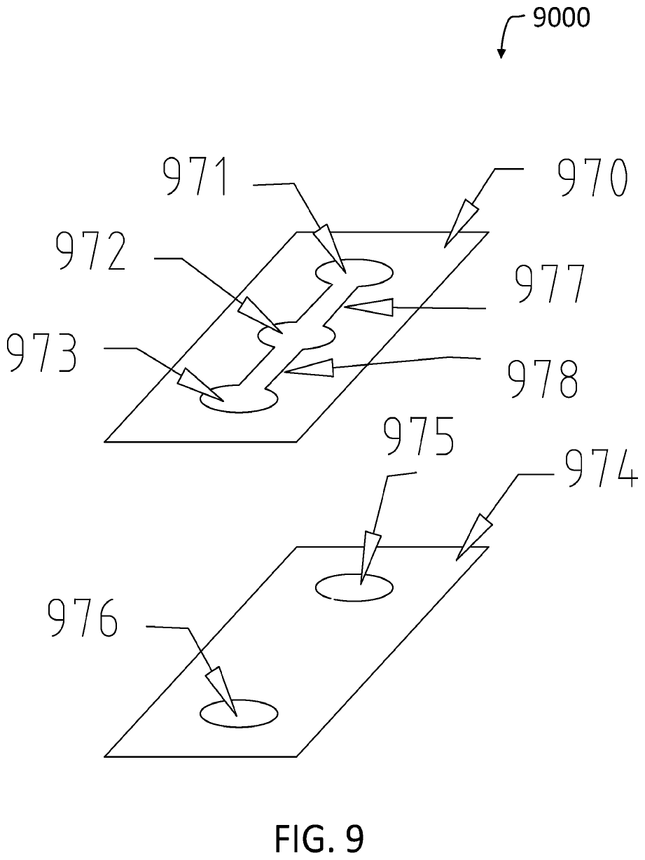

FIG. 9 shows a non-limiting embodiment of a fluidic device comprising a splitting layer. The splitting layer may be referred to elsewhere herein as a first layer, a second layer, a third layer, a fourth layer, a fifth layer, and/or a sixth layer. In FIG. 9, the fluidic device 9000 comprises a 2-channel splitting layer 970 and a vertical transport layer 974.

In the 2-channel splitting layer 970 shown in FIG. 9, the 2-channel splitting layer 970 comprises a central sample addition region 972, a first channel 977, a second channel 978, a first split sample region 971, and a second split sample region 973. The first channel 977 places the first split sample region 971 in fluidic communication with the central sample addition region 972, and the second channel 978 places the second split sample region 973 in fluidic communication with the central sample addition region 972.

The vertical transport layer 974 shown in FIG. 9 comprises a first vertical transport region 975 and a second vertical transport region 976. Fluidic devices having the design shown in FIG. 9 may comprise a vertical transport layer as shown in any one of FIGS. 2 and 4-7 further comprising a second vertical transport region. In some embodiments, each vertical transport region is in fluidic communication with a split sample region (e.g., the first vertical transport region may be in fluidic communication with the first split sample region, the second vertical transport region may be in fluidic communication with the second split sample region). Fluidic communication may be accomplished by contact between the split sample region and the vertical transport region. It is also possible for a split sample region and a vertical transport region to be in fluidic communication and for the split sample region to be disposed above the vertical transport region and at least partially laterally overlap the vertical transport region.

Fluidic devices comprising the layers shown in FIG. 9 may also comprise one or more further layers described elsewhere herein but not depicted in FIG. 9 (e.g., a lateral transport layer, a sample collection layer, a wash layer, a blotting layer).

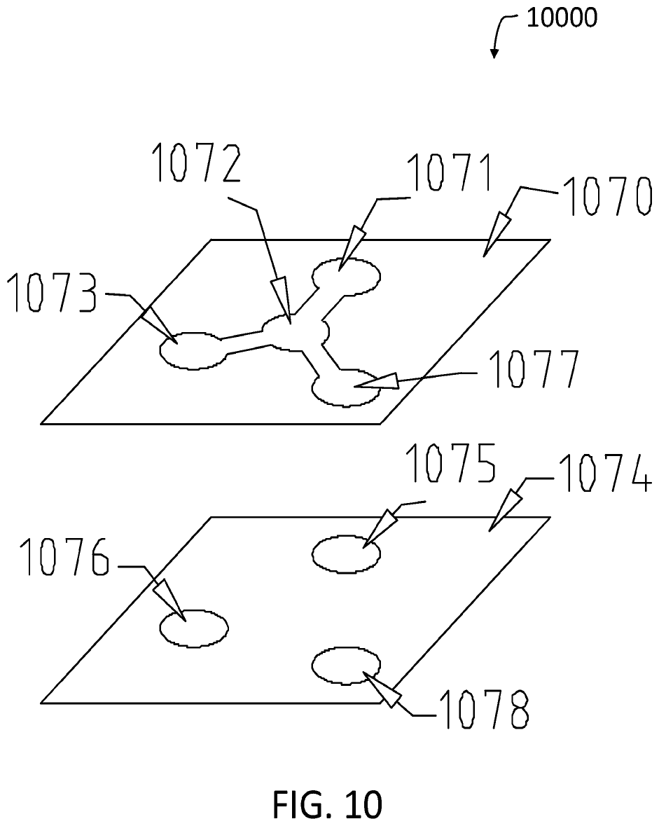

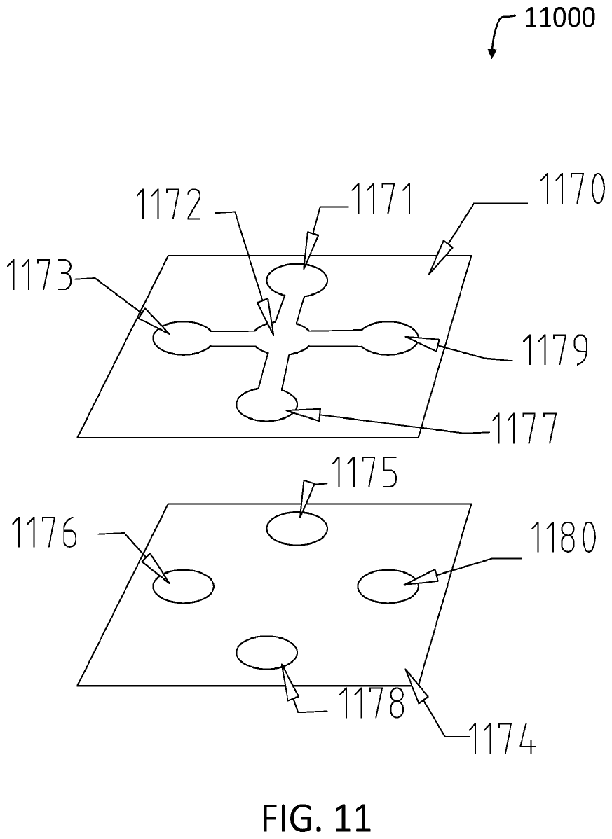

FIG. 10 illustrates a fluidic device 10000 comprising a 3-channel splitting layer 1070 and a vertical transport layer 1074. FIG. 11 illustrates a fluidic device 11000 comprising a 4-channel splitting layer 1170 and a vertical transport layer 1174. Fluidic devices having the designs shown in FIGS. 10 and 11 may comprise splitting layers as shown in FIG. 9 that further comprise additional channels and split sample regions. Similarly, fluidic devices having the designs shown in FIGS. 10 and 11 may comprise vertical transport layers as shown in any one of FIGS. 2 and 4-7 that further comprise additional vertical transport regions. Fluidic devices comprising the layers shown in FIG. 9 may also comprise one or more further layers described elsewhere herein but not depicted in FIG. 9 (e.g., a lateral transport layer, a sample collection layer, a wash layer, a blotting layer).

In some embodiments, the devices in FIGS. 9-11 may be configured to receive a fluid comprising a plurality of cells in the central addition region. The sample received by the central addition region may flow through the channels in the splitting layer to the split sample regions. The fluid sample received at each split sample region may flow vertically through the split sample region to an associated vertical transport region in the vertical transport layer positioned therebeneath (e.g., a vertical transport region disposed beneath the split sample region, a vertical transport region in fluidic communication with the split sample region).

The fluidic devices depicted in FIGS. 9-11 may be advantageous, for example, for performing multiplexing in a fluidic device. For example, such fluidic devices could be employed to split a fluid introduced into the fluidic device into multiple different aliquots and then to perform different operations (e.g., different types of cell labeling, cell capture in different locations) on different aliquots.

As described above, in some embodiments, a fluidic device comprises one or more layers that comprise a porous, absorbent material. For instance, a fluidic device may comprise a vertical transport layer that comprises a porous, absorbent material, a lateral transport layer that comprises a porous, absorbent material, a sample collection layer that comprises a porous, absorbent material, a wash layer that comprises a porous, absorbent material, a blotting layer that comprises a porous, absorbent material, and/or a splitting layer that comprises a porous, absorbent material. When two or more layers comprise porous, absorbent materials, the porous, absorbent materials positioned in the different layers may be identical to each other or may differ from each other in one or more ways. For instance, some fluidic devices may comprise two layers that comprise porous, absorbent materials having differing median and/or mode pore sizes. Additionally, each layer comprising a porous, absorbent material may independently comprise a porous, absorbent material having one or more of the properties described below and elsewhere herein as possibly characterizing porous, absorbent materials.

Some porous, absorbent materials comprise pores that are interconnected. The interconnection may be two-dimensional (e.g., in the lateral directions only) and/or may be three-dimensional. Some porous, absorbent materials are configured to allow lateral and/or vertical transport of cells in a fluid through the material. This transport may occur through interconnected pores. Some porous, non-absorbent materials do not allow for lateral and/or vertical transport of cells through the material. As an example, some porous, non-absorbent materials may be configured to allow the flow of an aqueous fraction of a fluid therethrough, but not allow the flow of cells positioned in the fluid therethrough.

Porous, absorbent materials described herein may have a variety of designs. In some embodiments, a fluidic device comprises a porous, absorbent material that is a fibrous material. The fibrous material may be a non-woven material, or may be a woven material. The fibers may have a variety of suitable diameters and distributions of diameters, and, if woven, may be woven in a variety of suitable weaves. One example of a suitable non-woven material is a paper.

In some embodiments, a fluidic device comprises a porous, absorbent material that is a cellulose-based material. The cellulose-based material may comprise cellulose derived from wood (e.g., it may be a wood-based material), cellulose derived from cotton (e.g., it may be a cotton-based material), and/or nitrocellulose. The cellulose-based material may comprise fibers formed from a cellulose-based material. For instance, the porous, absorbent material be a cellulose-based paper. A wide variety of commercially available cellulose-based papers may be employed, such as those manufactured by Whatman (e.g., Whatman CF12, Whatman 3 MM), those manufactured by Ahlstrom (e.g., 226, Munktell TFN). Further non-limiting examples of cellulose-based papers include WypAll X70 Wipers, WypAll L40 General-Purpose Wipers, WypAll L10 Utility Wipers, Professional Wypall X60 Wipers, Bounty Basic, Clever coffee paper filters, Viva Strong and Soft, and Bounty Duratowel.

In some embodiments, a porous, absorbent material comprises a synthetic material, glass, and/or a ceramic. It is also possible for a porous, absorbent material to comprise fibers comprising comprises a synthetic material, glass, and/or a ceramic. Non-limiting examples of suitable synthetic materials include poly(ether sulfone), polyesters, polysulfone, polycarbonate, polyvinylidene fluoride, polyamide, cellulose acetate, polytetrafluoroethylene (Teflon), polypropylene, polyethylene, and nylons. In some embodiments, a second porous, absorbent material comprises a synthetic material. Further non-limiting examples of suitable synthetic porous, absorbent materials include TX409 absorbond polyester wipers, Vectra Premium Polyester Wipers, ValuSeal 100% Polyester Knit Wipe, and Leukosorb membranes.

In some embodiments, a porous, absorbent material comprises both natural and synthetic materials, for example, of cellulose and polyester. Non-limiting examples of porous, absorbent materials comprising both natural and synthetic materials include TX1109 TechniCloth II nonwoven wipers, Professional Wypall X90 cloth wipers, and ProWipe750 non-woven polypropylene/cellulose.

In some embodiments, a porous, absorbent material may be chemically modified. The chemical modification may modulate the physiochemical properties of the porous, absorbent material. For example, in some instances an affinity agent may be chemically bonded to the porous, absorbent material (e.g., to fibers present in the porous, absorbent material). A variety of suitable techniques may be employed to bond the affinity agent to the porous, absorbent material. For example, polymerizable agents, such as amine monomers (e.g., 2-aminoethyl methacrylate) can be chemically bound to polymers present in a porous, absorbent material (e.g., polypropylene, polytetrafluoroethylene, etc.) using an electron-beam. As another example, amine-containing and/or hydroxyl-containing compounds can be bound to some porous, absorbent materials comprising polyester by performing a conjugation reaction (e.g., EDC/NHS coupling to form an amide, isothiocyanate coupling to form a thiourea, etc.). The conjugation reaction may be performed after a plasma treatment of the polyester material, a process that is believed to release reactive surface groups (e.g., hydroxyl groups and/or carboxylic acid groups).

In some embodiments, a porous, absorbent material may have a variety of suitable porosities. The porosity of the porous, absorbent material may be greater than or equal to 40 vol %, greater than or equal to 50 vol %, greater than or equal to 60 vol %, greater than or equal to 70 vol %, greater than or equal to 80 vol %, or greater than or equal to 90 vol %. The porosity of the porous, absorbent material may be less than or equal to 95 vol %, less than or equal to 90 vol %, less than or equal to 80 vol %, less than or equal to 70 vol %, less than or equal to 60 vol %, or less than or equal to 50 vol %. Combinations of the above-referenced ranges are also possible (e.g., greater than or equal to 40 vol % and less than or equal to 95 vol %, greater than or equal to 60 vol % and less than or equal to 95 vol %, or greater than or equal to 80 vol % and less than or equal to 95 vol %). Other ranges are also possible. The porosity of a porous, absorbent material may be determined by mercury intrusion porosimetry.

In some embodiments, a porous, absorbent material comprises pores having a median pore size that is advantageous for one or more reasons. As one example, in some embodiments, a porous, absorbent material comprises pores having a median pore size that is sufficiently large to allow for lateral and/or vertical transport of some cells therethrough. For instance, the median pore size may be larger than the size of the cells to be laterally and/or vertically transported through the porous, absorbent material. It is also possible for a porous, absorbent material to comprise pores having a median pore size that is too small to allow for lateral and/or vertical transport of some cells therethrough. As an example, the median pore size may be smaller than the size of the cells that are not to be laterally and/or vertically transported through the porous, absorbent material. In some embodiments, a porous, absorbent material comprises pores having a median pore size large enough to allow some types of cells to be laterally and/or vertically transported therethrough and small enough to prevent some types of cells from being laterally and/or vertically transported therethrough. In some embodiments, a porous, absorbent material comprises pores having a median pore size too small to allow the lateral or vertical transport of any cells therethrough. Such porous, absorbent materials may be suitable for trapping cells.

In some embodiments, a porous, absorbent material has a median pore size of greater than or equal to 0.8 microns, greater than or equal to 0.9 microns, greater than or equal to 1 micron, greater than or equal to 2 microns, greater than or equal to 3 microns, greater than or equal to 5 microns, greater than or equal to 7.5 microns, greater than or equal to 10 microns, greater than or equal to 12 microns, greater than or equal to 15 microns, greater than or equal to 18 microns, greater or equal to 20 microns, greater than or equal to 30 microns, greater than or equal to 50 microns, greater than or equal to 75 microns, greater than or equal to 100 microns, greater than or equal to 200 microns, greater than or equal to 400 microns, greater than or equal to 500 microns, or greater than or equal to 750 microns. In some embodiments, a porous, absorbent material comprises pores with a median pore size of less than or equal to 1 mm, less than or equal to 750 microns, less than or equal to 500 microns, less than or equal to 400 microns, less than or equal to 200 microns, less than or equal to 100 microns, less than or equal to 75 microns, less than or equal to 50 microns, less than or equal to 30 microns, less than or equal to 20 microns, less than or equal to 18 microns, less than or equal to 15 microns, less than or equal to 12 microns, less than or equal to 10 microns, less than or equal to 7.5 microns, less than or equal to 5 microns, less than or equal to 3 microns, less than or equal to 2 microns, less than or equal to 1 micron, or less than or equal to 0.9 microns. Combinations of the above-referenced ranges are also possible (e.g., greater than or equal to 0.8 microns and less than or equal to 1 mm, greater than or equal to 0.8 microns and less than or equal to 12 microns, greater than or equal to 15 microns and less than or equal to 1 mm). Other ranges are also possible.

The median pore size of a porous, absorbent material may be determined by mercury intrusion porosimetry.

When a fluidic device comprises two or more porous, absorbent materials, each porous, absorbent material may independently have a median pore size in one or more of the above-referenced ranges.

In some embodiments, a porous, absorbent material comprises pores having a mode pore size that is advantageous for one or more reasons. As one example, in some embodiments, a porous, absorbent material comprises pores having a mode pore size that is sufficiently large to allow for lateral and/or vertical transport of some cells therethrough. For instance, the mode pore size may be larger than the size of the cells to be laterally and/or vertically transported through the porous, absorbent material. It is also possible for a porous, absorbent material to comprise pores having a mode pore size that is too small to allow for lateral and/or vertical transport of some cells therethrough. As an example, the mode pore size may be smaller than the size of the cells that are not to be laterally and/or vertically transported through the porous, absorbent material. In some embodiments, a porous, absorbent material comprises pores having a mode pore size large enough to allow some types of cells to be laterally and/or vertically transported therethrough and small enough to prevent some types of cells from being laterally and/or vertically transported therethrough. In some embodiments, a porous, absorbent material comprises pores having a mode pore size too small to allow the lateral or vertical transport of any cells therethrough. Such porous, absorbent materials may be suitable for trapping cells.

In some embodiments, a porous, absorbent material comprises pores with a mode pore size of greater than or equal to greater than or equal to 0.8 microns, greater than or equal to 0.9 microns, greater than or equal to 1 micron, greater than or equal to 2 microns, greater than or equal to 3 microns, greater than or equal to 5 microns, greater than or equal to 7.5 microns, greater than or equal to 10 microns, greater than or equal to 12 microns, greater than or equal to 15 microns, greater than or equal to 18 microns, greater or equal to 20 microns, greater than or equal to 30 microns, greater than or equal to 50 microns, greater than or equal to 75 microns, greater than or equal to 100 microns, greater than or equal to 200 microns, greater than or equal to 400 microns, greater than or equal to 500 microns, or greater than or equal to 750 microns. In some embodiments, a porous, absorbent material comprises pores with a mode pore size of less than or equal to 1 mm, less than or equal to 750 microns, less than or equal to 500 microns, less than or equal to 400 microns, less than or equal to 200 microns, less than or equal to 100 microns, less than or equal to 75 microns, less than or equal to 50 microns, less than or equal to 30 microns, less than or equal to 20 microns, less than or equal to 18 microns, less than or equal to 15 microns, less than or equal to 12 microns, less than or equal to 10 microns, less than or equal to 7.5 microns, less than or equal to 5 microns, less than or equal to 3 microns, less than or equal to 2 microns, less than or equal to 1 micron, or less than or equal to 0.9 microns. Combinations of the above-referenced ranges are also possible (e.g., greater than or equal to 0.8 microns and less than or equal to 1 mm, greater than or equal to 0.8 microns and less than or equal to 12 microns, greater than or equal to 15 microns and less than or equal to 1 mm). Other ranges are also possible.

The median pore size of a porous, absorbent material may be determined by mercury intrusion porosimetry.

When a fluidic device comprises two or more porous, absorbent materials, each porous, absorbent material may independently have a mode pore size in one or more of the above-referenced ranges.

In some embodiments, a porous, absorbent material has a pore size distribution that is advantageous for one or more reasons. As one example, in some embodiments, a porous, absorbent material comprises a relatively low number of pores having a size that is too small to allow for lateral and/or vertical transport of some cells therethrough. For instance, the porous, absorbent material may comprise a relatively low number of pores that are smaller than the size of the cells to be laterally and/or vertically transported through the porous, absorbent material. It is also possible for a porous, absorbent material to comprise a relatively large number of pores having a size that is too small to allow for lateral and/or vertical transport of some cells therethrough. As an example, the porous, absorbent material may comprise an appreciable number of pores having a size that is smaller than the size of the cells that are not to be laterally and/or vertically transported through the porous, absorbent material. In some embodiments, a porous, absorbent material comprises pores having a relatively large number of pores having a size large enough to allow some types of cells to be laterally and/or vertically transported therethrough and small enough to prevent some types of cells from being laterally and/or vertically transported therethrough. In some embodiments, a porous, absorbent material comprises pores having a relatively large number of pores having a size too small to allow the lateral or vertical transport of any cells therethrough. Such porous, absorbent materials may be suitable for trapping cells.

In some embodiments, a porous, absorbent material has a relatively low number of pores having a size of less than 10 microns. In some embodiments, less than or equal to 2%, less than or equal to 1.75%, less than or equal to 1.5%, less than or equal to 1.25%, less than or equal to 1%, less than or equal to 0.75%, less than or equal to 0.5%, less than or equal to 0.25%, less than or equal to 0.1%, less than or equal to 0.01%, or less than or equal to 0.001% of the pores in a porous, absorbent material have a size of less than 10 microns. In some embodiments, greater than or equal to 0%, greater than or equal to 0.001%, greater than or equal to 0.01%, greater than or equal to 0.1%, greater than or equal to 0.25%, greater than or equal to 0.5%, greater than or equal to 0.75%, greater than or equal to 1%, greater than or equal to 1.25%, greater than or equal to 1.5%, or greater than or equal to 1.75% of the pores in a porous, absorbent material have a size of less than 10 microns. Combinations of the above-referenced ranges are also possible (e.g., greater than or equal to 0% and less than or equal to 2%). Other ranges are also possible.

The percentage of pores having a size of less than 10 microns in a porous, absorbent material may be determined by mercury intrusion porosimetry.

When a fluidic device comprises two or more porous, absorbent materials, each porous, absorbent material may independently have a percentage of pores having a pore size of less than 10 microns in one or more of the above-referenced ranges.

In some embodiments, a porous, absorbent material has a relatively low number of pores having a size of less than 20 microns. In some embodiments, less than or equal to 5%, less than or equal to 4%, less than or equal to 3%, less than or equal to 2%, less than or equal to 1.75%, less than or equal to 1.5%, less than or equal to 1.25%, less than or equal to 1%, less than or equal to 0.75%, less than or equal to 0.5%, less than or equal to 0.25%, less than or equal to 0.1%, less than or equal to 0.01%, or less than or equal to 0.001% of the pores in a porous, absorbent material have a size of less than 20 microns. In some embodiments, greater than or equal to 0%, greater than or equal to 0.001%, greater than or equal to 0.01%, greater than or equal to 0.1%, greater than or equal to 0.25%, greater than or equal to 0.5%, greater than or equal to 0.75%, greater than or equal to 1%, greater than or equal to 1.25%, greater than or equal to 1.5%, greater than or equal to 1.75%, greater than or equal to 2%, greater than or equal to 3%, or greater than or equal to 4% of the pores in a porous, absorbent material have a size of less than 20 microns. Combinations of the above-referenced ranges are also possible (e.g., greater than or equal to 0% and less than or equal to 5%). Other ranges are also possible.

The percentage of pores having a size of less than 20 microns in a porous, absorbent material may be determined by mercury intrusion porosimetry.

When a fluidic device comprises two or more porous, absorbent materials, each porous, absorbent material may independently have a percentage of pores having a pore size of less than 20 microns in one or more of the above-referenced ranges.

In some embodiments, a porous, absorbent material has a relatively low number of pores having a size of less than 30 microns. In some embodiments, less than or equal to 10%, less than or equal to 7.5%, less than or equal to 5%, less than or equal to 4%, less than or equal to 3%, less than or equal to 2%, less than or equal to 1.75%, less than or equal to 1.5%, less than or equal to 1.25%, less than or equal to 1%, less than or equal to 0.75%, less than or equal to 0.5%, less than or equal to 0.25%, less than or equal to 0.1%, less than or equal to 0.01%, or less than or equal to 0.001% of the pores in a porous, absorbent material have a size of less than 30 microns. In some embodiments, greater than or equal to 0%, greater than or equal to 0.001%, greater than or equal to 0.01%, greater than or equal to 0.1%, greater than or equal to 0.25%, greater than or equal to 0.5%, greater than or equal to 0.75%, greater than or equal to 1%, greater than or equal to 1.25%, greater than or equal to 1.5%, greater than or equal to 1.75%, greater than or equal to 2%, greater than or equal to 3%, greater than or equal to 4%, greater than or equal to 5%, or greater than or equal to 7.5% of the pores in a porous, absorbent material have a size of less than 30 microns. Combinations of the above-referenced ranges are also possible (e.g., greater than or equal to 0% and less than or equal to 10%). Other ranges are also possible.

The percentage of pores having a size of less than 30 microns in a porous, absorbent material may be determined by mercury intrusion porosimetry.

When a fluidic device comprises two or more porous, absorbent materials, each porous, absorbent material may independently have a percentage of pores having a pore size of less than 30 microns in one or more of the above-referenced ranges.

In some embodiments, a porous, absorbent material may, upon exposure to a fluid (e.g., a fluid comprising cells), wick the fluid (and, possibly cells therein) into the porous, absorbent material and/or wick the fluid through the porous, absorbent material. When layers comprising channels comprise a porous, absorbent material positioned in the channels, the porous, absorbent material may wick fluid (and, possibly cells therein) into the channels therein and/or through the channels therein. In some embodiments, a fluid may flow (and, possibly cells therein) into and/or through a porous, absorbent material due to capillarity (capillary action) and/or by wicking. In some embodiments, a fluid (and, possibly cells therein) may flow into and/or through a porous, absorbent material due to capillarity. In some embodiments, a porous, absorbent material will, upon exposure to a fluid (e.g., a fluid comprising cells), transport the fluid (and, possibly cells therein) into the interior of the porous, absorbent material (i.e., the fluid may penetrate into the interior of the material in which the pores are positioned, such as into the interior of fibers making up a porous, absorbent material that comprises fibers). In some embodiments, a porous, absorbent material will, upon exposure to a fluid, experience an increase in mass due to the fluid (and, possibly cells therein) absorbed therein. It should be understood that some layers comprising porous absorbent materials may have one or more of the properties described above with respect to porous, absorbent materials.

In some embodiments, a method described herein, and/or one or more steps of a method described herein is passive. For example, in some embodiments, a method is done solely with the use of gravity and/or capillary action. In some embodiments, a method is done without the use of centrifugation, electricity, and/or an external field (e.g., acoustic, electric, and/or magnetic). For example, in some embodiments, a fluid (e.g., a fluid comprising cells) is added to a fluidic device and then the fluidic device transports the fluid (and, possibly cells therein) without further action (that is, the fluid and/or cells within the fluid are transported purely from gravity and capillary action).

In some embodiments, a fluidic device comprises a layer that comprises a material that is porous but not absorbent. For instance, in some embodiments, a sample collection layer comprises a material that is porous but not absorbent. When two or more layers comprise porous, non-absorbent materials, the porous, non-absorbent materials positioned in the different layers may be identical to each other or may differ from each other in one or more ways. Additionally, each layer comprising a porous, non-absorbent material may independently comprise a porous, non-absorbent material having one or more of the properties described below and elsewhere herein as possibly characterizing porous, non-absorbent materials.

Some porous, non-absorbent materials comprise pores that are interconnected. The interconnection may be two-dimensional (e.g., in the lateral directions only) and/or may be three-dimensional. Some porous, non-absorbent materials are configured to allow lateral and/or vertical transport of cells in a fluid through the material. This transport may occur through interconnected pores. Some porous, non-absorbent materials do not allow for lateral and/or vertical transport of cells through the material. As an example, some porous, non-absorbent materials may be configured to allow the flow of an aqueous fraction of a fluid therethrough, but not allow the flow of cells positioned in the fluid therethrough.

It is also possible for a porous, non-absorbent material to comprise pores that are laterally isolated pores. For instance, in some embodiments, a sample collection layer comprises a porous, non-absorbent material that comprises laterally isolated pores. Such pores may allow for vertical transport of a fluid (and, possibly cells therein) vertically through the porous, non-absorbent layer (e.g., through one or more vertical transport regions therein) while not allowing for lateral transport of the fluid.

In some embodiments, a non-absorbent porous material comprises a hydrophobic polymer membrane and/or a phase inversion membrane (e.g., a water-insoluble polymer that has processed into a solid porous material). Such materials can be made, for example, by precipitation from the vapor phase, precipitation by controlled evaporation, thermally induced phase separation, and/or immersion precipitation.

In some embodiments, a porous, non-absorbent material comprises pores having a median pore size that is advantageous for one or more reasons. As one example, a porous, non-absorbent material may comprise pores having a median pore size too small to allow the lateral or vertical transport of any cells therethrough. Such porous, non-absorbent materials may be suitable for trapping cells.

In some embodiments, a porous, non-absorbent material has a median pore size is at least 0.8 microns, at least 0.9 microns, at least 1 micron, at least 2 microns, at least 3 microns, at least 4 microns, at least 5 microns, at least 6 microns, at least 7 microns, at least 8 microns, at least 9 microns, at least 10 microns, or at least 11 microns. In some embodiments, a porous, non-absorbent material has a median pore size is at most 12 microns, at most 11 microns, at most 10 microns, at most 9 microns, at most 8 microns, at most 7 microns, at most 6 microns, at most 5 microns, at most 4 microns, at most 3 microns, at most 2 microns, at most 1 micron, or at most 0.9 microns. Combinations of the above-referenced ranges are also possible (e.g., at least 0.9 microns and at most 12 microns). Other ranges are also possible.

The median pore size of a porous, non-absorbent material may be determined by mercury intrusion porosimetry.

When a fluidic device comprises two or more porous, non-absorbent materials, each porous, absorbent material may independently have a median pore size in one or more of the above-referenced ranges.

In some embodiments, a porous, non-absorbent material comprises pores having a mode pore size that is advantageous for one or more reasons. As one example, a porous, non-absorbent material may comprise pores having a mode pore size too small to allow the lateral or vertical transport of any cells therethrough. Such porous, non-absorbent materials may be suitable for trapping cells.

In some embodiments, a porous, non-absorbent material has a mode pore size is at least 0.8 microns, at least 0.9 microns, at least 1 micron, at least 2 microns, at least 3 microns, at least 4 microns, at least 5 microns, at least 6 microns, at least 7 microns, at least 8 microns, at least 9 microns, at least 10 microns, at least 11 microns, or at least 12 microns. In some embodiments, a porous, non-absorbent material has a mode pore size is at most 12 microns, at most 11 microns, at most 10 microns, at most 9 microns, at most 8 microns, at most 7 microns, at most 6 microns, at most 5 microns, at most 4 microns, at most 3 microns, at most 2 microns, at most 1 micron, or at most 0.9 microns. Combinations of the above-referenced ranges are also possible (e.g., at least 0.9 microns and at most 12 microns). Other ranges are also possible.

The mode pore size of a porous, non-absorbent material may be determined by mercury intrusion porosimetry.

When a fluidic device comprises two or more porous, non-absorbent materials, each porous, absorbent material may independently have a mode pore size in one or more of the above-referenced ranges.

In some embodiments, the device comprises a blotting layer. In such embodiments, the blotting layer may comprise an absorbent material (e.g., a porous, absorbent material described elsewhere herein) and/or may be relatively thick. Some blotting layers are configured to wick and/or absorb any excess fluid from a layer to which it is adjacent. Such a design may allow the blotting layer to wick excess fluid from a wash layer (or any layer beneath which the blotting layer is positioned). This may be beneficial, for instance, in the case where it is desirable to separate cells from a fluid. Wicking away the excess fluid may assist with this separation. As another example, wicking excess fluid from a wash layer may be desirable for fluidic devices in which affinity-labeling is performed. Solubilized affinity agents in a fluid may obscure any signal arising from labeled cells, which may reduce the device sensitivity. A blotting layer in fluidic communication with a wash region in a wash layer may wick fluid from a sample collection region through the wash region, which may affect the signal arising from the cells trapped in the sample collection layer.

In some embodiments, a fluidic device comprises a blotting layer that comprises a non-woven material, such as a paper. In some embodiments, a blotting layer comprises a Sham Wow.

The absorbency of a blotting layer may be greater than or equal to 300 microliters/cm, greater than or equal to 400 microliters/cm², or greater than or equal to 500 microliters/cm². The absorbency of a blotting layer may be less than or equal to 600 microliters/cm², less than or equal to 500 microliters/cm², or less than or equal to 400 microliters/cm². Combination of the above referenced ranges are also possible (e.g., greater than or equal to 300 microliters/cm² and less than or equal to 600 microliters/cm²). Other ranges are also possible.

As used herein, the absorbency of a blotting layer is determined by weighing the blotting layer, saturating it in DI water for 30 seconds, weighing it again, determining the difference between the second weight and the first weight (i.e., the weight of the DI water absorbed), and then converting this weight to a volume of water (e.g., microliters) using the density of DI water at room temperature. The volume of DI water absorbed is then normalized by dividing by the surface area (e.g., cm²) of the blotting layer.

When a fluidic device comprises two or more blotting layers, each blotting layer may independently have an absorbency in one or more of the above-referenced ranges.

In some embodiments, a fluidic device comprises a splitting layer. In some embodiments, a splitting layer comprises a central sample addition region. The central sample addition region may be configured to receive a fluid sample. The central sample addition region may also be in fluidic communication with two or more channels. In some embodiments, the central sample addition region is in fluidic communication with at least 2 channels, at least 3 channels, at least 4 channels, at least 5 channels, at least 6 channels, at least 7 channels, at least 8 channels, at least 9 channels, or at least 10 channels (and, in some embodiments, at most 11 channels, at most 10 channels, at most 9 channels, at most 8 channels, at most 7 channels, at most 6 channels, at most 5 channels, at most 4 channels, and/or at most 3 channels).

In some embodiments, a channel in a splitting layer may place a central sample addition region in fluidic communication with a split sample region. For example, a splitting layer may comprise a central sample addition region in fluidic communication with two or more channels, each of which places the central sample addition region in fluidic communication with a split sample region.

As described above, in some embodiments, a fluidic device comprises a splitting layer disposed on a vertical transport layer. In such embodiments, the vertical transport layer may comprise a plurality of vertical transport regions. For example, in some embodiments, the receiving layer may comprise a first vertical transport region, a second vertical transport region, a third vertical transport region, a fourth vertical transport region, and so on. In some embodiments, the vertical transport regions in a vertical transport layer are fluidically isolated from (i.e., they are not in fluidic communication with) each other through the vertical transport layer. In such embodiments, these vertical transport regions may be in fluidic communication with regions in adjacent layers. For example, a splitting layer comprising two split sample regions disposed on a vertical transport layer may comprise a first split sample region in fluidic communication with a first vertical transport region and a second split sample region in fluidic communication with a second vertical transport layer.

In some embodiments, the device comprises a filter. Some such filters are porous. In some embodiments, a fluidic device comprises a filter that is a track-etched membrane. Without wishing to be bound by theory, track-etched membranes may be formed by bombarding a polymer thin film with high energy particles. The high energy particles may cause “tracks” (i.e., pores) to be etched into the thin film. The resultant membranes may comprise pores having relatively uniform sizes and/or densities. Some suitable track-etched membranes comprise a polyester, such as for example, a polycarbonate or a polyethylene terephthalate. Further non-limiting examples of materials that may be included in track-etched membranes include poly(vinylidene fluoride) (PVDF) and poly(imide) (PI).

The fluidic devices described herein may comprise filters comprising laterally isolated pores and/or may comprise filters comprising interconnected pores. Some filters comprise pores configured to prevent lateral and/or vertical transport of cells within a fluid through the filter while permitting the lateral and/or vertical transport of another portion of the fluid (e.g., an aqueous fraction).

In some embodiments, a filter comprises pores with a median pore size of greater than or equal to 10 microns, greater than or equal to 12 microns, greater than or equal to 14 microns, greater than or equal to 16 microns, or greater than or equal to 18 microns. In some embodiments, a filter comprises pores with a median pore size of less than or equal to 20 microns, less than or equal to 18 microns, less than or equal to 16 microns, less than or equal to 14 microns, or less than or equal to 12 microns. Combinations of the above-referenced ranges are also possible (e.g., greater than or equal to 10 microns and less than or equal to 20 microns). Other ranges are also possible.

The median pore size of a filter may be determined by mercury intrusion porosimetry.

When a fluidic device comprises two or more filters, each filter may independently have a median pore size in one or more of the above-referenced ranges.

In some embodiments, a filter comprises pores with a mode pore size of greater than or equal to 10 microns, greater than or equal to 12 microns, greater than or equal to 14 microns, greater than or equal to 16 microns, or greater than or equal to 18 microns. In some embodiments, a filter comprises pores with a mode pore size of less than or equal to 20 microns, less than or equal to 18 microns, less than or equal to 16 microns, less than or equal to 14 microns, or less than or equal to 12 microns. Combinations of the above-referenced ranges are also possible (e.g., greater than or equal to 10 microns and less than or equal to 20 microns). Other ranges are also possible.

The mode pore size of a filter may be determined by mercury intrusion porosimetry.

When a fluidic device comprises two or more filters, each filter may independently have a mode pore size in one or more of the above-referenced ranges.

In some embodiments, a filter is positioned between a vertical transport layer and a lateral transport layer. Additionally or alternatively, a filter may be positioned a sample collection layer and a lateral transport layer.

Some aspects of the disclosure relate to transporting a fluid comprising a plurality of cells through one or more portions of a fluidic device. Non-limiting examples of fluids that may be so transported include fluids of biological origin, such as blood (e.g., whole blood) and fluids derived from blood (e.g., plasma), spinal fluid, cerebrospinal fluid, urine, bone marrow, tissue biopsies, and milk. A variety of cells may be present in the fluids described herein, such as blood cells (e.g., red blood cells, white blood cells), immune cells, cells that produce myeloperoxidase, and/or cells that produce leukocyte esterase.

In some embodiments, a fluid that is transported through one or more portions of a fluidic device is whole blood. In some embodiments, the fluid is a blood sample that is diluted with water and/or a buffer solution. In some embodiments, the fluid is a blood sample that is undiluted blood from a subject. In some embodiments, the subject is an animal, such as a mammal. In some embodiments, the subject is a human. In some embodiments, the fluid sample comprises peripheral blood mononuclear cells (PBMCs). In some embodiments, one or more portions of the fluidic device comprises an anti-coagulant (e.g., ethylenediaminetetraacetic acid (EDTA) and/or heparin), such as a dried anti-coagulant. In some embodiments, the anti-coagulant may be positioned in a vertical transport layer (e.g., in a vertical transport region) and/or in a lateral transport layer (e.g., in a channel therein).

Some embodiments are directed to fluidic devices in which one or more reagents are stored. Reagents stored in a fluidic device (e.g., in a vertical transport region, in a sample collection region) may be stored therein in a variety of ways. Non-limiting examples of ways that reagents may be stored in the fluidic device include being adsorbed onto a material present in the fluidic device (e.g., fibers in a vertical transport region, fibers in a sample collection region), absorbed into a material present in the fluidic device (e.g., fibers in a vertical transport region, fibers in a sample collection region), and/or in a gel present in the fluidic device (e.g., in a vertical transport region, in a sample collection region). In some embodiments, reagents may be deposited onto one or more fibers in the fluidic device (e.g., one or more fibers in a vertical transport region, a sample collection region, and/or a channel). Some reagents may be stored in the fluidic device as solids. The solids may be present in a matrix, such as a matrix comprising a protein (e.g., bovine serum albumin) and/or a sugar (e.g., sucralose, trehalose). In some embodiments, one or more reagents stored in a fluidic device (e.g., as solids) may be reconstituted and/or dissolved in a fluid flowing therethrough. For example, a fluid may flow through a vertical transport region comprising one or more reagents, and at least a portion of the one or more reagents may dissolve in the fluid. In some embodiments, cells trapped on a sample collection region may react with one or more reagents disposed in the sample collection region.

In some embodiments, a fluidic device comprises a reagent that is an affinity agent, such as, for example, an antibody. In some instances, the antibody may be configured to bind to human cells. Non-limiting examples of possible antibodies include anti-CD45, anti-CD11b, anti-CD16, anti-CD66b, anti-CD193, anti-FCeR1a, anti-CD63, anti-CD203c, anti-CD14, anti-CD15, anti-CD68, anti-CD83, anti-XCR1, anti-CLEC9A, anti-CD1c, SIRPa, anti-HLA-DR, anti-CD80, anti-iNOS, anti-CD163, anti-CD206, anti-CD3, anti-CD56, anti-CD4, anti-CD5, anti-CD8, anti-CD13, anti-CD20, anti-CD30, anti-CD34, anti-CD19, anti-human IgG1, anti-human IgG2, anti-human IgG3, anti-human IgG4, anti-human IgA1, anti-human IgA. It should be understood, however, that in other embodiments, antibodies derived from other species (e.g., mouse, rat, donkey, monkey, etc.) and/or of different immunoglobin classes (e.g., IgM, IgA, IgE, and IgD) are also possible.

In other embodiments, a fluidic device comprises an affinity agent other than an antibody. Non-limiting examples of such affinity agents include a single-chain antibody fragment, a Fab fragment, a protein, a peptide, an aptamer, an oligonucleotide, a carbohydrate, a nanobody, and a nanoparticle.

In some embodiments, an affinity agent comprises a cellulose binding domain (CBD). The cellulose binding domain may be capable of binding to a cellulose-based material, such as paper. In some embodiments, a fluidic device comprises a cellulose binding domain that is conjugated to streptavidin and/or avidin. It is also possible for a fluidic device to comprise a cellulose binding domain that is conjugated to a Protein A (ProA-CBD). Cellulose binding domains may be advantageous, for example, for binding multiple affinity agents with different cell targets to a porous, absorbent material that is cellulose-based.

In some embodiments, an affinity agent may be stored in a layer (e.g., vertical transport layer, lateral transport layer, sample collection layer, etc.). Affinity agents may be stored within one region in a layer or within two or more regions in a layer (e.g., vertical transport regions, sample reception regions, etc.). In some embodiments, the affinity agents may be stored within one channel or within two or more channels in a layer (e.g., a channel in a lateral transport layer).

In some embodiments, an affinity agent is conjugated to detection agent, such as a chromogen. In some cases, the detection agent may permit visualization of the cell(s) to which the affinity agent is attached. In some embodiments, a detection agent and/or a chromogen comprises an enzyme, such as horseradish peroxidase, alkaline phosphatase, and/or glucose oxidase. In some cases, a detection agent and/or a chromogen comprises a dye and/or a dyed particle, such as, for example, a nanoparticle, a nanodot, and/or a quantum dot. In some embodiments, a detection agent comprises a fluorophore. In some embodiments, the fluorophore comprises alexa fluor 350, alexa fluor 405, alexa fluor 488, alexa fluor 532, alexa fluor 546, alexa fluor 555, alexa fluor 561, alexa fluor 568, alexa fluor 594, alexa fluor 647, alexa fluor 660, alexa fluor 680, alexa fluor 700, alexa fluor 750, DyLight 350, DyLight 405, DyLight 488, DyLight 550, DyLight 594, DyLight 633, DyLight 650, DyLight 680, DyLight 755, DyLight 800, BODIPY FL, coumarin, Cy3, Cy5, fluorescein, Oregon green, pacific blue, pacific green, pacific orange, PE-cyanine7, PerCp-Cyanine5.5, tetramethyl rhodamine, and/or Texas red.

In some embodiments, a fluidic device comprises a reagent that is a detection reagent. Non-limiting examples of suitable detection reagents include myeloperoxidase-specific substrates and leukocyte esterase-specific substrates.

In some embodiments, a detection agent may be stored in a layer (e.g., sample collection layer). Detection reagents may be stored within one region in a layer or within two or more regions in a layer (e.g., sample collection regions).

As described above, some embodiments relate to methods. Further details regarding some methods are provided below.

Some methods comprise flushing a fluidic device with a cleansing fluid (e.g., phosphate buffered saline) after a fluid comprising cells has finished flowing through the device. This may be accomplished while some cells are still positioned at one or more locations in the fluidic device, such as a filter and/or a sample reception region. For instance, a fluidic device may be flushed with a cleansing fluid after cells have been trapped in the device but before the trapped cells are further analyzed. Flushing a fluidic device with a cleansing fluid may help to remove any unbound affinity agents located in the device prior to the flushing, which may reduce any background signal associated with the unbound affinity agents. In some embodiments, the volume of cleansing fluid added to the device is at least 25 microliters, at least 50 microliters, at least 75 microliters, at least 100 microliters, at least 125 microliters, at least 150 microliters, or at least 175 microliters. In some embodiments, the volume of cleansing fluid added to the device is no more than 200 microliters, no more than 175 microliters, no more than 150 microliters, no more than 125 microliters, no more than 100 microliters, no more than 75 microliters, or no more than 50 microliters.

In some embodiments, the method comprises disassembling a fluidic device and/or removing one or more regions and/or layers from a fluidic device. In some embodiments, the region or layer of interest may be removed by biopsy punch and/or by way of peeling. In some embodiments, a region or layer of interest (e.g., a sample collection region) may be configured to be removed from the fluidic device. For example, the region and/or layer to be removed may comprise cuts, gaps, or perforations surrounding the region and/or layer that advantageously facilitate removal of the region and/or layer. In some embodiments, a blotting layer may comprise boundary features such as tabs, loops, and/or holes to facilitate removal of the sample collection region. In some embodiments, a sample collection region is configured to be removed using tweezers.

In some embodiments, a method comprises incubating a removed region and/or layer (e.g., a removed sample collection region) in a developer solution to produce a signal, such as an optical signal. The signal may subsequently be detected (e.g., optically and/or visually). Non-limiting examples of suitable optical signals include colorimetric signals, signals generated by chemiluminescence, and/or signals generated by fluorescence. In some embodiments, the signal may be employed to determine a concentration of immune cells.

A variety of suitable developer solutions may be employed. In some embodiments, a developer solution that comprises a chromogen is employed. For example, affinity agents conjugated to horseradish peroxidase detection agents may be developed using chromogens such as 3-Amino-9-Ethylcarbazole (AEC), 3,3′-Diaminobenzidine (DAB), 3,3′,5,5′-Tetramethylbenzidine (TMB), and/or Stay Yellow. As another example, affinity agents conjugated to alkaline phosphatase detection reagents may be developed using chromogens such as StayGreen, 5-bromo-4-chloro-3indolyl-phosphate-p-toluidine/tetranitroblue tetrazolium (BCIP/TNBT), and 5-bromo-4-chloro-3indolyl-phosphate-p-toluidine/nitrotetrazolium blue chloride (BCIP/NBT).

In some embodiments, a developer solution comprises a dye. Exemplary dyes that may be included in developer solutions include hematoxylin and cosin (i.e., H&E stain), Van Gieson's stain, Toluidine blue, alcian blue, giesma stain, potassium manganate, silver solution, gold chloride, nuclear fast red, Biebrich scarlet acid fuchsin, phosphotungstic acid, phosphomolybdic acid, light green, and orange G solution.

In some embodiments, a developer solution comprises a fluorescent dye. Fluorescent dies may illuminate cell membranes. Exemplary fluorescent dyes include DiO, DiI, Cytopainter, DAPI, propidium iodide, calcein AM, and BOBO-3 iodide.

In some embodiments, a developer solution may comprise one or more primary antibodies configured to bind to one or more cell populations on a layer and/or region. In some embodiments, a developer solution comprises a secondary antibody configured to bind to the primary antibody. In some embodiments, the secondary antibody is conjugated to a detection agent. In some embodiments, the primary antibody is conjugated to a detection agent.

In some embodiments, a developer solution comprises a reactive substrate. In some cases, the substrate may react with a myeloperoxidase, which may be produced by one or more cell types (e.g., neutrophils and monocytes) on the region and/or layer of interest (e.g., sample collection region). Accordingly, in some embodiments, the developer solution comprises a myeloperoxidase-specific substrate. The myeloperoxidase-specific substrate may be part of a kit, purchased from a commercial vendor (e.g., Celltechgen), or synthesized by those skilled in the art. For example, without wishing to be bound by theory, exposing cells expressing myeloperoxidase to a solution comprising peroxide (H₂O₂) and chloride ions (Cl⁻) can produce hypochlorous acid (HCIO), which can be subsequently reacted with taurine to form a taurine choloramine. Taurine chloroamine can react with a chromophore, such as TNB, resulting in a colorless product DTNB. The MPO activity may then be determined by the rate of disappearance of the TNB color. It should also be understood that other substrates (and kits), may also be used to quantify the myeloperoxidase activity (e.g. ELISA kits) or the activity of other enzymes of interest.

In some cases, a method comprises quantifying leukocyte esterase activity as a general marker for total white blood cell count. This may be accomplished, for example, by quantifying the rate at which ethyl butyrate (a substrate) is converted into butyric acid and ethanol. Other reactive substrates may also be used, for example, to quantify the leukocyte esterase concentration, such as for example, an enzyme-linked immunosorbent assay (ELISA). In some embodiments, any form of ELISA may be used including direct ELISA, indirect ELISA, sandwich ELISA, and competitive ELISA, and the like.

In some embodiments, a method comprises quantifying a produced signal. In some embodiments, the produced signal (e.g., a signal produced by incubating a sample collection region in a developer solution) may be measured using any suitable metric, such as for example, color intensity or the percentage of the total area of the region and/or layer occupied by the signal (% Area). In some embodiments, a portion of a fluidic device (e.g., a sample collection layer, a sample collection region) may be visually inspected and qualitatively measured using a grading system. For example, sample collection regions in which % Arca is less than 10% may be given a score of 1, regions in which the % Area is between 10% and 30% may be given a score of 2, regions in which the % Area is between 30% and 50% may be given a score of 3, regions in which the % Area is between 50% and 70% may be given a score of 4, and regions in which the % Area is greater than 70% may be given a score of 5. However, it should be understood that the above example is non-limiting and that any metric and/or grading system known to those of skill in the art may be used to qualitatively measure the target metric.

In some embodiments, a signal may be quantitatively measured using imagining software, such as for example, ImageJ or MetaMorph (see Example 1). For example, microphotographs of a region and/or layer of interest (e.g., a sample collection region) may be collected using, for example, an optical microscope equipped with a camera. The microphotographs can be imported into an imaging software (e.g. ImageJ) and the color threshold adjusted such that pixels are assigned only to the areas where a the signal is present. In some embodiments, the processed image can be converted into a binary image and the % Area of the pixels quantified. In other embodiments, the % Area may be used to generate calibration curves that permit quantification of the target cell concentration.

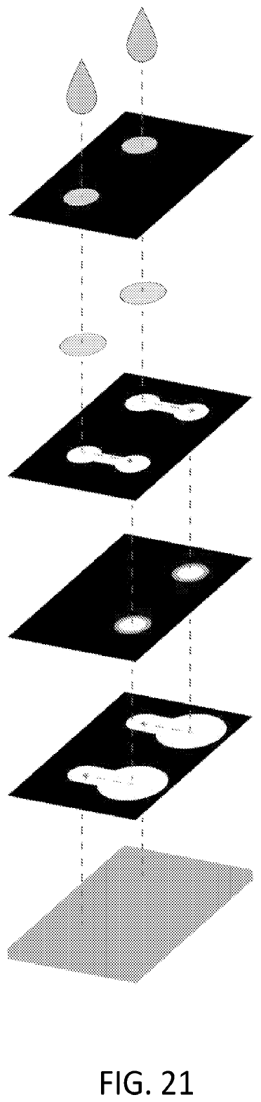

In some embodiments, a method comprises running multiple analyses on the same fluid sample (e.g., multiplexing). For example, in some cases it may be desirable to simultaneously affinity-label and enrich a target cell type within a fluid sample in situ (e.g., removing monocytes from a fluid comprising peripheral blood mononuclear cells, thereby enriching a lymphocyte fraction for in situ affinity labeling and quantification). As such, in some embodiments, a filter, such as a polycarbonate track-etched (PCTE) filter, may be placed between a vertical transport layer and a lateral transport layer, such that the filter is in fluidic communication with a vertical transport region and a first sample reception region (see FIG. 21). In some embodiments, a filter may have a median and/or mode pore sizes that selectively captures the cell of interest. For example, monocytes (˜20 microns) may be separated from lymphocytes (˜6-9 microns) by passing an aliquot of PBMCs through a PCTE filter with a median pore size of 10 microns. In some embodiments, both the filter and the sample collection region are removed and treated with the appropriate developer solution to permit enumeration of both cell types.

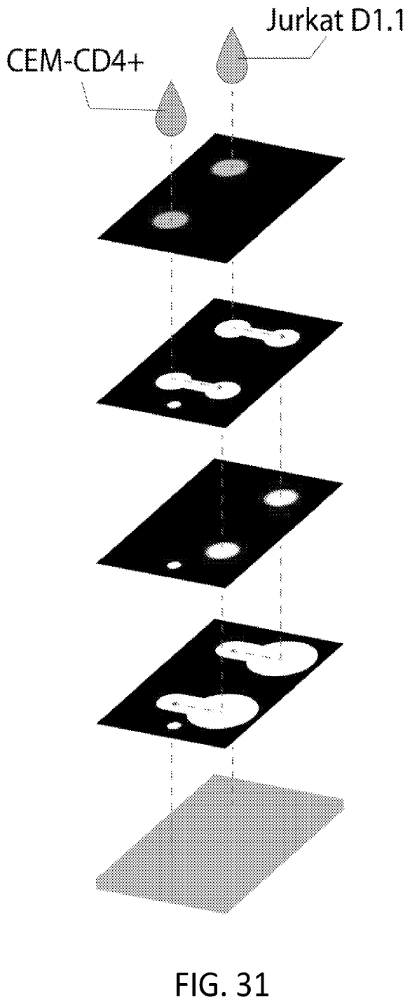

As another example, in some cases it may be desirable to enrich or deplete a target cell of interest using a biomarker (e.g., as opposed to size-exclusion, see FIG. 31). As such, in some embodiments, at least one region (e.g., vertical transport region) and/or layer (e.g. vertical transport layer) may comprise a cellulose binding domain conjugated to either Protein-A or streptavidin. As described elsewhere herein, the cellulose binding domain may be configured to bind to cellulose-based materials, such as paper filters. Protein-A, in turn, may be configured to bind to the fragment crystallizable region (Fc region) of an antibody (i.e., the tail end) with a particularly high affinity for IgGs isotypes (e.g., IgG1, IgG2, and IgG4). Streptavidin, on the other hand, may be configured to bind to any moiety conjugated to a biotin molecule. In this way, antibodies (e.g., with an IgG isotype) and/or biotin-conjugated moieties (e.g. peptides, proteins, aptamers, nucleotides, polynucleotides, nanoparticles) targeting one or more cells types may be introduced into the device and used to either enrich or deplete a fluid sample.

In some embodiments, a vertical transport region, within a vertical transport layer, comprises a Protein-A-cellulose binding domain (see FIG. 31, top layer). In some embodiments, a vertical transport region is configured to receive a fluid sample comprising an affinity agent, for example, an anti-CD4 antibody (Isotype IgG2b, kappa). In some embodiments, the Protein A in the Protein-A-cellulose binding domain is configured to bind to the affinity agent (e.g., anti-CD14), such that the affinity agent (e.g., anti-CD14) becomes immobilized within the vertical transport region. In some embodiments, the vertical transport region is configured to receive a second fluid comprising cells, for example, peripheral blood mononuclear cells (PBMCs). Such a configuration may allow monocytes and dendritic cells to be captured in the vertical transport region (i.e., depleting these cells types from the fluid sample) while permitting the remaining enriched lymphocytes (e.g., T cells, B cells, and NK cells) to flow through the device until they are captured in the sample collection region.

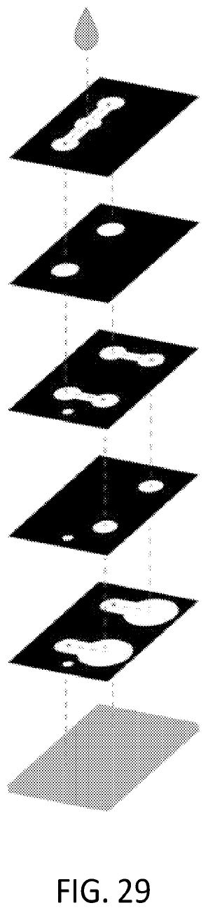

In some embodiments, a method comprises performing a full differential white blood cell count on a fluid comprising white blood cells. This may be accomplished by splitting a single sample into four different channels, and employing each channel to enumerate a cell type of interest (e.g., lymphocytes, monocytes, cosinophils, and/or basophils). Fluidic devices suitable for performing this method may comprise a splitting layer comprising four channels disposed on a vertical transport layer comprising four fluidically isolated vertical transport regions (see FIG. 11 and FIG. 29). In some embodiments, a fluid may be added to the splitting layer (e.g., via the central sample addition region). Addition of the fluid to the splitting layer may drive the fluid into the first split sample region, the second split sample region, the third split sample region, and the fourth split sample region. In some embodiments, the fluid may flow vertically from the first sample split region into the first vertical transport region, from the second sample split region into the second vertical transport region, from the third sample split region into the third vertical transport region, and from the fourth sample split region into the fourth vertical transport region. In some embodiments, the first vertical transport region comprises an affinity agent (e.g., anti-CD14 for labeling of monocytes); in some embodiments, the second vertical transport region comprises a second affinity agent (e.g., anti-CD3 for labeling lymphocytes); in some embodiments, the third vertical transport region comprises a third affinity agent (e.g., anti-CD193 for labeling of cosinophils); in some embodiments, the fourth vertical transport region comprises a fourth affinity agent (e.g., anti-CD63 for labeling of basophils). In some embodiments, flow of the fluid from the sample split region into a vertical transport region solubilizes the affinity agent disposed in the vertical transport region, which may allow the affinity agent to bind to the target cell.

Some methods comprise quantifying cell subtypes (e.g. monocytes, lymphocytes, neutrophils, cosinophils, activated basophils versus quiescent basophils). As such, in some embodiments, a device comprises a filter positioned between a splitting layer and a vertical transport layer and/or between a sample collection layer and a lateral transport layer. Monocytes may become trapped with the filter (e.g., when using a PCTE filter comprising pores having median and/or mode pore sizes of 10 microns), thus allowing an affinity agent targeting activated basophils to be embedded in one of the sample receiving regions of the receiving layer.

Aspects of the disclosure relate to methods of fluidic device fabrication and/or assembly. As described above, fluidic devices described herein may comprise one or more channels (e.g., in one or more of the layers described herein). The channels may be open channels (e.g., the channels may be open along two sides, or open along one side), or the channels may be enclosed. The channels may have a variety of suitable dimensions. In some embodiments, one or more channels are present in a layer, and the channel extends through the thickness of the layer. In other words, some channels may have the same thickness as the layers in which they are positioned. In some embodiments, one or more channels may have dimensions that aid in metering of a fluid sample. The channel(s) may have a volume, dimension, and/or shape that promotes flow of a desired volume of the fluid sample therein and/or therethrough.

A fluidic device may comprise a channel with a thickness or height of greater than or equal to 50 microns, greater than or equal to 100 microns, greater than or equal to 200 microns, greater than or equal to 400 microns, greater than or equal to 500 microns, greater than or equal to 800 microns, greater than or equal to 1000 microns, greater than or equal to 1200 micron, greater than or equal to 1400 microns, greater than or equal to 1600 micron, or greater than or equal to 1800 microns. The fluidic device may comprise a channel with a thickness or height of less than or equal to 2000 microns, less than or equal to 1600 microns, less than or equal to 1400 microns, less than or equal to 1200 microns, less than or equal to 1000 microns, less than or equal to 800 microns, less than or equal to 500 microns, less than or equal to 400 microns, less than or equal to 200 microns, or less than or equal to 100 microns. Combinations of the above-referenced ranges are also possible (e.g., greater than or equal to 50 microns and less than or equal to 2000 microns, greater than or equal to 100 microns and less than or equal to 1000 microns, or greater than or equal to 200 microns and less than or equal to 800 microns). Other ranges are also possible.

Channels in fluidic devices may have a variety of suitable widths. In some embodiments, a fluidic device comprises a channel with a width of greater than or equal to 50 microns, greater than or equal to 100 microns, greater than or equal to 200 microns, greater than or equal to 500 microns, greater than or equal to 1000 microns, greater than or equal to 2000 microns, greater than or equal to 3000 microns, greater than or equal to 4000 microns, greater than or equal to 5000 microns, greater than or equal to 6000 microns, or greater than or equal to 7000 microns. The fluidic device may comprise a channel with a width of less than or equal to 8000 microns, less than or equal to 7000 microns, less than or equal to 6000 microns, less than or equal to 5000 microns, less than or equal to 4000 microns, less than or equal to 3000 microns, less than or equal to 2000 microns, less than or equal to 1000 microns, less than or equal to 500 microns, less than or equal to 200 microns, or less than or equal to 100 microns. Combinations of the above-referenced ranges are also possible (e.g., greater than or equal to 2000 microns and less than or equal to 8000 microns, or greater than or equal to 4000 microns and less than or equal to 6000 microns). Other ranges are also possible.

Channels in fluidic devices may have a variety of suitable aspect ratios (i.e., ratios of the channel length to the channel width). In some embodiments, a fluidic device comprises a channel with an aspect ratio of greater than or equal to 2:1, greater than or equal to 3:1, greater than or equal to 4:1, greater than or equal to 5:1, greater than or equal to 6:1, greater than or equal to 7:1, greater than or equal to 8:1, or greater than or equal to 9:1. The fluidic device may comprise a channel with an aspect ratio of less than or equal to 10:1, less than or equal to 9:1, less than or equal to 8:1, less than or equal to 7:1, less than or equal to 6:1, less than or equal to 5:1, less than or equal to 4:1, or less than or equal to 3:1. Combinations of the above-referenced ranges are also possible (e.g., greater than or equal to 2:1 and less than or equal to 10:1). Other ranges are also possible.

In some embodiments, a fluidic device comprises a channel with a volume of greater than or equal to 5 microliters, greater than or equal to 10 microliters, greater than or equal to 20 microliters, greater than or equal to 30 microliters, or greater than or equal to 40 microliters. The fluidic device may comprise a channel with a volume of less than or equal to 50 microliters, less than or equal to 40 microliters, less than or equal to 30 microliters, less than or equal to 20 microliters, or less than or equal to 10 microliters. Combinations of the above-referenced ranges are also possible (e.g., greater than or equal to 5 microliters and less than or equal to 50 microliters). Other ranges are also possible. In some embodiments, a channel comprises a sample region with a volume in one or more of the ranges described above (e.g., a channel may comprise a sample region with a volume of greater than or equal to 5 microliters and less than or equal to 50 microliters, greater than or equal to 10 microliters and less than or equal to 40 microliters, or greater than or equal to 20 microliters and less than or equal to 30 microliters).

As described above, fluidic devices described herein may comprise one or more regions (e.g., in one or more of the layers described herein). The regions may be open regions, or the regions may be enclosed. The regions may have a variety of suitable dimensions. In some embodiments, one or more regions are present in a layer, and the region extends through the thickness of the layer. In other words, some regions may have the same thickness as the layers in which they are positioned. In some embodiments, the shape of the region may be any geometry selected from the group consisting of regular polygons (e.g. a two dimensional plane shape with straight sides where all sides and angles are the same), irregular polygons (e.g. a two dimension plane shape with straight sides where the sides and angles are not the same), circles, and/or ellipses.

A fluidic device may comprise a region with a thickness or height of greater than or equal to 50 microns, greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, or greater than or equal to 900 microns. The fluidic device may comprise a region with a thickness or height of less than or equal to 1000 microns, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, or less than or equal to 100 microns. Combinations of the above-referenced ranges are also possible (e.g., greater than or equal to 50 microns and less than or equal to 1000 microns, greater than or equal to 100 microns and less than or equal to 900 microns, or greater than or equal to 200 microns and less than or equal to 800 microns). Other ranges are also possible.

Regions in fluidic devices may have a variety of suitable widths. In some embodiments, a fluidic device comprises a region with a width of greater than or equal to 50 microns, greater than or equal to 100 microns, greater than or equal to 200 microns, greater than or equal to 500 microns, greater than or equal to 1000 microns, greater than or equal to 2 millimeters, greater than or equal to 4 millimeters, greater than or equal to 6 millimeters, greater than or equal to 8 millimeters, or greater than or equal to 1 centimeter. The fluidic device may comprise a region with a width of less than or equal to 2 centimeters, less than or equal to 1 centimeter, less than or equal to 8 millimeters, less than or equal to 6 millimeters, less than or equal to 4 millimeters, less than or equal to 2 millimeters, less than or equal to 1000 microns, less than or equal to 500 microns, less than or equal to 200 microns, or less than or equal to 100 microns. Combinations of the above-referenced ranges are also possible (e.g., greater than or equal to 50 microns and less than or equal to 2 centimeters, or greater than or equal to 100 microns and less than or equal to 8000 millimeters). Other ranges are also possible.

Regions in fluidic devices may have a variety of suitable aspect ratios (i.e., ratios of the region length to the region width). In some embodiments, a fluidic device comprises a region with an aspect ratio of greater than or equal to 1:1, greater than or equal to 2:1, greater than or equal to 3:1, greater than or equal to 4:1, greater than or equal to 5:1, greater than or equal to 6:1, greater than or equal to 7:1, greater than or equal to 8:1, or greater than or equal to 9:1. The fluidic device may comprise a region with an aspect ratio of less than or equal to 10:1, less than or equal to 9:1, less than or equal to 8:1, less than or equal to 7:1, less than or equal to 6:1, less than or equal to 5:1, less than or equal to 4:1, less than or equal to 3:1, less than or equal to 2:1, or less than or equal to 1:1. Combinations of the above-referenced ranges are also possible (e.g., greater than or equal to 2:1 and less than or equal to 10:1). Other ranges are also possible.

In some embodiments, a fluidic device comprises a region with a volume of greater than or equal to 5 microliters, greater than or equal to 10 microliters, greater than or equal to 20 microliters, greater than or equal to 30 microliters, or greater than or equal to 40 microliters. The fluidic device may comprise a region with a volume of less than or equal to 50 microliters, less than or equal to 40 microliters, less than or equal to 30 microliters, less than or equal to 20 microliters, or less than or equal to 10 microliters. Combinations of the above-referenced ranges are also possible (e.g., greater than or equal to 5 microliters and less than or equal to 50 microliters). Other ranges are also possible.

Some of the layers comprising regions and/or channels may also comprise a porous, absorbent material. In some embodiments, the region and/or channel may pass through a porous, absorbent material. Regions and/or channels may be formed in a layer and/or a porous, absorbent material (e.g., a layer comprising a porous, absorbent material) by a variety of suitable methods. By way of example, a barrier impermeable to a fluid may be infiltrated into portions of the layer and/or porous, absorbent material to define channels and/or regions therein. This may be accomplished by, e.g., printing (e.g., wax printing, 3D-printing) and/or pattern transfer methods (e.g., by use of photoresists and/or UV-curable materials). The fluid to which the barrier is impermeable (e.g., a fluid comprising cells, one or more components of such a fluid) may, upon entering a channel and/or region defined by an impermeable barrier, be confined to portions of the layer and/or porous, absorbent material of which it can flow through without crossing the impermeable barrier (e.g., channels and/or regions in fluidic communication with the channel and/or region defined by the impermeable barrier).

Barriers impermeable to a variety of fluids may be employed. In some embodiments, the fluid to which a barrier is impermeable is an aqueous fluid, such as a fluid of biological origin. Non-limiting examples of fluids of biological origin include blood (e.g., whole blood) and fluids derived from blood (e.g., plasma), cerebrospinal fluid, tissue biopsies, and milk. The barrier impermeable to a fluid may comprise a variety of suitable materials, non-limiting examples of which include waxes, polymers, and hydrophobic materials (e.g., hydrophobic waxes, hydrophobic polymers, other hydrophobic materials).

In some embodiments, one or more layers of the device are adhered to one or more layers. For example, in some embodiments, a device comprises an adhesive that adheres a vertical transport layer to a lateral transport layer. As another example, in some embodiments, a device comprises an adhesive that adheres a lateral transport layer to a sample collection layer. In some embodiments, all of the layers in a fluidic device are adhered together by adhesives. In some embodiments, one or more layers are permanently adhered or integrally connected to one or more layers. In some embodiments, one or more layers are reversibly adhered to one or more layers. Examples of suitable methods of adhering layers include double-sided adhesive (e.g., double-sided medical adhesive), liquid adhesive, sonic welding, and/or compression. In some embodiments, one or more layers are adhered to one or more layers (and/or a support structure) with an adhesive. Examples of suitable adhesives include double-sided adhesive (e.g., double-side medical adhesive), compression tape, 3M brand adhesive, and/or Flexcon brand adhesive. In some embodiments, the adhesive is placed on a surface of a layer. In some embodiments, the adhesive is placed around the perimeter of a layer where it contacts another layer (or substrate) to adhere it to the other layer (or substrate). In some embodiments, the adhesive (e.g., between two layers, or between a layer and the substrate) provides a full seal (e.g., a seal around the entire perimeter of the layer through which fluid cannot pass).

In some embodiments, the adhesive is applied manually. In some embodiments, the adhesive is applied with a laser cutter, ultrasonic welding, and/or UV curing. In some embodiments, the adhesive has a low tack. In some embodiments, one or more layers is adhered to one or more layers in such a way that they cannot be pulled apart manually without damaging one or more of the layers. For example, in some embodiments, one layer is adhered to another such that they cannot be pulled apart manually without damaging one or more of the layers. In some embodiments, one or more layers is adhered to one or more layers in such a way that they can be pulled apart manually without damaging one or more of the layers. In some embodiments, one layer is adhered to another in such a way that they can be pulled apart manually, without having to use so much force that it will disrupt either or both layers (e.g., creating mess or contamination), but such that the layers do not come apart during use (e.g., do not come apart during separation of a blood sample).

In some embodiments, a fluidic device comprises a cover layer. Advantageously, the cover layer may enclose and/or protect the fluidic device in which it is positioned. It may be impermeable to one or more fluids to be introduced into the fluidic device, may be impermeable to one or more components of an environment external to the fluidic device, may strengthen the fluidic device, and/or may decrease the tendency of the fluidic device to be damaged during handling.

A fluidic device may comprise a cover layer that is the uppermost layer and/or a cover layer that is the lowermost layer. The cover layer may further comprise one or more openings, which may be in fluidic communication with one or more features of a layer to which it is adjacent. For instance, an uppermost cover layer may comprise one or more openings in fluidic communication with a central sample addition region and/or a vertical transport region. In some embodiments, a cover layer lacks openings and prevents fluidic communication between a layer to which it is adjacent an environment external to the fluidic device through the cover layer. For instance, a lowermost cover layer may seal the bottom of the fluidic device from direct fluidic communication with an environment beneath the fluidic device.

The cover layers described herein typically comprise materials that are relatively impermeable to a variety of fluids (e.g., aqueous fluids), relatively impermeable to a variety of gases (e.g., gases in the ambient environment), and/or relatively scuff and/or abrasion resistant. In some embodiments, a fluidic device comprises a cover layer that is a laminating sheet (such as a Fellowes laminating sheet) and/or an adhesive film. When laminating sheets and/or adhesive films are employed, the fluidic device may be assembled by laminating the other layers thereof in between two laminating sheets and/or adhesive films.

In some embodiments, the article comprises a support structure. In some embodiments, the support structure comprises a plastic, an acrylic, and/or a metal. In some embodiments, the support structure is a plastic scaffold or an acrylic scaffold.

In some embodiments, the support structure is adjacent one or more layers. In some embodiments, the support structure is adjacent the lateral transport layer, vertical transport layer, sample collection layer, and/or any other layer positioned in the fluidic device. In some embodiments, the support structure is in direct contact with one or more layers. In some embodiments, the support structure is in direct contact with the lateral transport layer, vertical transport layer, sample collection layer, and/or any other layer positioned in the fluidic device.

In some embodiments, a support structure is adhered to one or more layers (e.g., a vertical transport layer). Examples of suitable means to adhere (e.g., the support structure to one or more layers) are discussed elsewhere herein (e.g., in reference to adhering one layer to another layer). In some embodiments, a support structure is not adhered to one or more layers (e.g., not adhered to any layers). For example, in some embodiments, a portion of a fluidic device (e.g., the lateral transport layer, the vertical transport layer, the sample collection layer, and/or any other layer in the fluidic device) sits on the support structure.

In some embodiments, the support structure comprises a cavity. In some embodiments, the cavity is used for holding a portion of the fluidic device (e.g., the lateral transport layer, the vertical transport layer, the sample collection layer, and/or any other layer in the fluidic device). In some embodiments, the cavity is circular, oval, square, rectangular, and/or diamond shaped. In some embodiments, the cavity is of a similar shape as a cross-section (e.g., a horizontal cross-section) of a portion of the fluidic device (e.g., one or more of the layers therein). For example, in some embodiments, the cavity and/or the cross-section of a portion of the fluidic device (e.g., one or more layers) are both circular, oval, square, rectangular, and/or diamond shaped.

In some embodiments, the depth of the cavity is less than the thickness of the support structure, such that, when viewed from above, a layer of the support structure is present throughout the surface area of the support structure. In some embodiments, the cavity is configured such that a portion of the fluidic device (e.g., the lateral transport layer, the vertical transport layer, the sample collection layer, and/or any other layer therein) can sit inside the cavity. In some embodiments, the cavity is configured such that a portion of the article (e.g., the lateral transport layer, vertical transport layer, and/or third layer) can sit inside the cavity, with the bottom surface of the bottommost layer of the fluidic device in contact with the support structure.

In some embodiments, a cavity is present throughout the thickness of a support structure, such that, when viewed from above, the cavity is a hole in the support structure. In some embodiments, the cavity has different maximum horizontal dimensions at different thickness of the support structure. For example, in some embodiments, the cavity has a larger maximum horizontal dimension at one opening than at the other. In some embodiments, the larger maximum horizontal dimension at one opening is greater than or equal to the maximum horizontal dimension of a portion of the article (e.g., the third layer). In some embodiments, the smaller maximum horizontal dimension at the other opening is less than the maximum horizontal dimension of another portion of the fluidic device (e.g., a layer therein). In some embodiments, the cavity is configured such that a portion of the fluidic device (e.g., the lateral transport layer, the vertical transport layer, the sample collection layer, and/or any other layer therein) can sit inside the cavity. In some embodiments, the cavity is configured such that a portion of the fluidic device (e.g., the lateral transport layer, the vertical transport layer, the sample collection layer, and/or any other layer therein) can sit inside the cavity, but the bottom surface of the bottommost layer is not in contact with the support structure. In some embodiments, the cavity is configured such that a portion of the fluidic device (e.g., the lateral transport layer, the vertical transport layer, the sample collection layer, and/or any other layer in the fluidic device) can sit inside the cavity, but the bottom surface of the bottommost layer is not in contact with the support structure, such that the bottommost layer can be removed from the fluidic device through the bottom of the support structure (e.g., through the opening with the smaller maximum horizontal dimension), while the remaining portions of the fluidic device can remain in the support structure.

In some embodiments, the cavity is configured such that the height of the edges (e.g., circumference) of the cavity prevent a portion of the fluidic device (e.g., the lateral transport layer, the vertical transport layer, the sample collection layer, and/or any other layer in the fluidic device) from significant horizontal movement, but the portion of the fluidic device (e.g., the lateral transport layer, the vertical transport layer, the sample collection layer, and/or any other layer in the fluidic device) can still be picked up vertically. In some embodiments, the height of the edges of the cavity are greater than or equal to ⅕ the thickness of a layer (e.g., the bottommost layer), greater than or equal to ¼ the thickness of a layer (e.g., the bottommost layer), greater than or equal to ⅓ the thickness of a layer (e.g., the bottommost layer), greater than or equal to ½ the thickness of a layer (e.g., the bottommost layer), or greater than or equal to the thickness of a layer (e.g., the third layer). In some embodiments, the height of the edges of the cavity are less than or equal to 3 times the thickness of a layer (e.g., the bottommost layer), 2 times the thickness of a layer (e.g., the bottommost layer), the thickness of a layer (e.g., the bottommost layer), ½ the thickness of a layer (e.g., the bottommost layer), ⅓ the thickness of a layer (e.g., the bottommost layer), or ¼ the thickness of a layer (e.g., the bottommost layer). Combinations of these ranges are also possible (e.g., greater than or equal to ⅕ and less than or equal to 3 times the thickness of a layer (e.g., the bottommost layer)).

In some embodiments, a fluidic device may comprise one or more features designed to aid identification of the fluidic device and/or one or more samples contained therein. For instance, the fluidic device may comprise a QR code, which may be linked to an online database including one or more types of information, such as information about a patient from which samples on contained on the device have originate and/or information about a hospital and/or clinic used by the patient (and/or at which the fluidic device was used to obtain the samples). In some embodiments, a QR code may be used to improve tracking of the fluidic device.

The following examples are intended to illustrate certain embodiments of the present disclosure, but do not exemplify the full scope of the disclosure.

### Example 1

### Abstract

A fluidic device allowing for white blood cell (WBC) transport in both the lateral and vertical directions is described. This fluidic device comprises a coffee filter in two of the layers, a material that allows for this type of cell transport. Additionally, this Example describes labeling WBCs in situ with an enzyme-labeled affinity reagent and enumeration of WBCs according to their immunophenotype. Using two cultured leukocyte lines (Jurkat D1.1 T cells and MAVER-1 B cells), the specific, colorimetric enumeration of each target cell population is described across the physiological range for total lymphocytes, 1000-4000 cells μL⁻¹.

### Description

WBCs are larger than red blood cells, ranging from 7-20 μm in diameter. Due to the presence of a nucleus, WBCs are also believed to be less flexible than red blood cells. For adequate WBC transport in a porous material, it is believed that the diameters of the pores must be large enough to allow for WBCs to pass through them. The inclusion of lateral, directed flow in fluidic devices, such as patterned paper devices, may allow for many more opportunities in paper-based device design, including the ability to incorporate complex sample-processing (e.g., selective cell labeling), multiplexing, and/or to allow for proper mixing of any affinity-based labelling reagents in situ.

This Example describes fluidic devices including a porous material that allows for the transport of WBCs by wicking in both the vertical and lateral directions: a coffee filter. Such fluidic devices are believed to be capable of the controlled transport, labeling, and detection of intact WBCs. Using two leukocyte cell lines with complementary immunophenotypes, Jurkat D1.1 T cells (CD3+/CD19−) and MAVER-1 B cells (CD3−/CD19+), it is demonstrated that the exemplary fluidic devices described in this Example can detect and enumerate WBC subsets based on their immunophenotype. The fluidic devices described in this Example are capable of labeling and detecting an intact WBC. These fluidic devices device labels WBCs with a reporter-antibody conjugate to provide a specific, colorimetric readout that corresponds to cell count. The colorimetric signal readout can be visualized qualitatively by eye or scanned and analyzed for a quantitative readout. The fluidic devices described in this Example are believed to be suitable for point-of-care cytometry and/or performing hematology analyses.

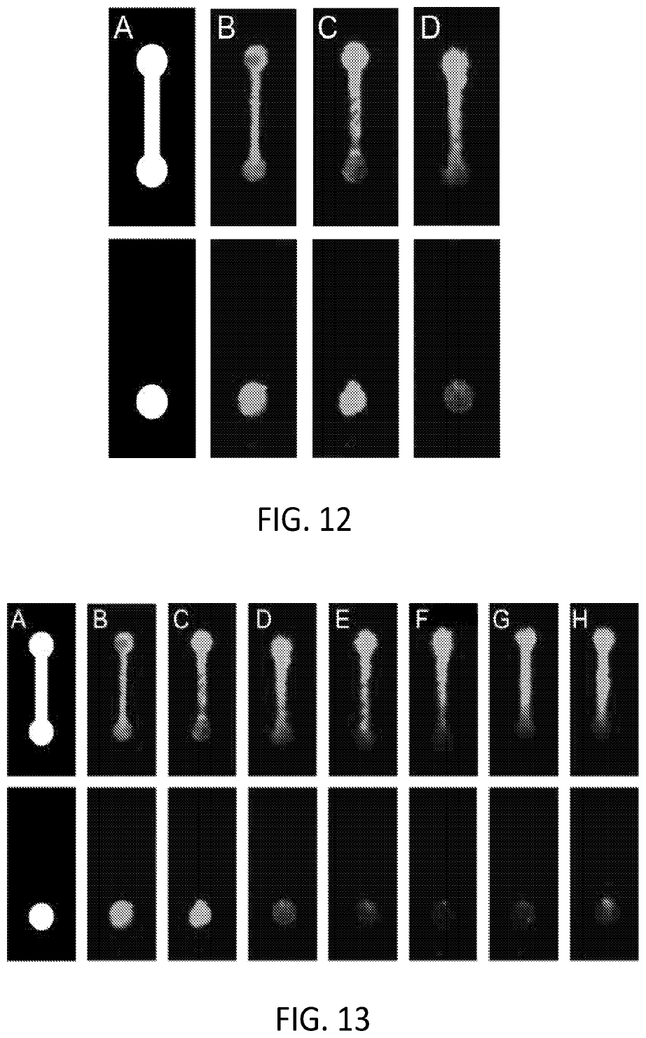

To build a fluidic device capable of vertical and lateral transport of WBCs, a broad material screen was performed. A material that would allow for the transport of WBCs in both the lateral and vertical directions was sought. It was believed that transport along a lateral channel would allow mixing of the cells with labelling reagents and vertical transport would allow cell movement to a cell collection layer for readout. Another goal was to reduce and/or eliminate capture based on the physical properties (e.g., size and charge) of the cells in order to promote cell transport through the fluidic device to the cell collection layer. The materials screened included paper towels (e.g., Bounty Basic), synthetic wipers (e.g., Technicloth), and coffee filters (Table 1). Each material was evaluated based on its ability to (i) maintain a patterned wax barrier and (ii) transport cells in both the lateral and vertical directions. To assess cell transport, two-layer fluidic devices were fabricated. These devices comprised (i) a test material with a patterned single channel (a lateral transport layer) and (ii) a membrane (polyether sulfone, PES) patterned with a circular sample collection region (a sample collection layer) (FIG. 12A). To track cell transport, a MAVER-1s (a B cell line) was stained with a general membrane fluorescent dye (DiI). The stained cells were added to one end of the patterned channel and their transport through the channel to the sample collection layer was visualized with a fluorescent scanner (Azure c400 Gel Imaging System). Visualization of fluorescent cells on the sample collection layer indicated cell transport (FIGS. 12B-C, which depict fluidic devices comprising sample transport layers formed from a Kimwipe and a Clever coffee filter, respectively). The absence of fluorescent cells or the presence of fluorescent cells that were not easily observed on the sample collection layer indicated no or poor cell transport, respectively (FIG. 12D, which depicts a fluidic device comprising a sample transport layer formed from Technicloth synthetic wipers, TX1109). Kimwipes and Clever coffee filters showed the most intense signal on the sample collection layer (FIGS. 13A-I, which depicts images from fluidic devices to which stained cells were not added (FIG. 13A), and fluidic devices comprising cell transport layers formed from a Kimwipe (FIG. 13B), a Clever coffee filter (FIG. 13C), a Technicloth synthetic wiper, TX 1109 (FIG. 13D), a Professional WypAll X60 Wiper (FIG. 13E), a Wypall X70 wiper (FIG. 13F), a DURX 670 non-woven polyester/cellulose wiper (FIG. 13G), a TX612 Technicloth nonwoven wiper (FIG. 13H), and Ahlstrom 55 filter paper (FIG. 13I)). Kimwipes were found to be fragile, which was observed to make handling and wax printing difficult.

TABLE 1

| | | Maintains wax | Transports |
|---|---|---|---|
| Material | Supplier | barrier? | WBCs? |
| WypAll X70 Wipers | Fisher Scientific | Y | Y |
| WypAll L40 General-Purpose Wipers | Fisher Scientific | N | — |
| WypAll L10 Utility Wipers | Fisher Scientific | N | — |
| Professional WypAll X60 Wipers | Fisher Scientific | Y | Y |
| Professional WyPall X90 Cloth Wipers | Fisher Scientific | N | — |
| TX1109 technicloth II nonwoven wipers | Fisher Scientific | Y | Y |
| Clever coffee paper filters | Amazon | Y | Y |
| TX409 absorbond polyester wipers | Fisher Scientific | N | — |
| TX612 technicloth nonwoven wipers | Fisher Scientific | Y | Y |
| Viva Strong and Soft | Amazon | N | — |
| Bounty Basic | Amazon | N | — |
| Bounty Duratowel | Amazon | N | — |
| Viva Vantage Strong and Scrubs | Amazon | N | — |
| Target 2-ply toilet paper | Target | N | — |
| Vectra Premium Polyester Wipers, TX1029 | Fisher Scientific | Y | N |
| Bounty Select-a-Size 2 × More Absorbent | Amazon | N | — |
| Paper Towels | | | |
| Cottonelle toilet paper | Amazon | Y | N |
| MicroPolx 1100 Sealed Edge Cleanroom | Fisher Scientific | Y | N |
| Wipers | | | |
| CleanWIPE Wipers HT4208-5 | VWR | N | — |
| CleanWIPE Wipers HT4211 | VWR | N | — |
| ProWipe 750 non-woven | VWR | N | — |
| polyproylene/cellulose | | | |
| DURX 670 non-woven polyester/cellulose | VWR | Y | Y |
| ValuSeal IonX Sealed Edge Cleanroom | VWR | Y | N |
| Laundered 100% Polyester Knit Wipe | | | |
| LabX 123 Low-linting pipers | VWR | Y | N |
| Kimwipe | Fisher Scientific | Y | Y |
| Lens paper | Fisher Scientific | Y | N |
| Singed Polyester Felt Filter Media Fabric | Amazon | N | — |
| Sheet 50 Micron | | | |
| Singed Polyester Felt Filter Media Fabric | Amazon | N | — |
| Sheet 25 Micron | | | |

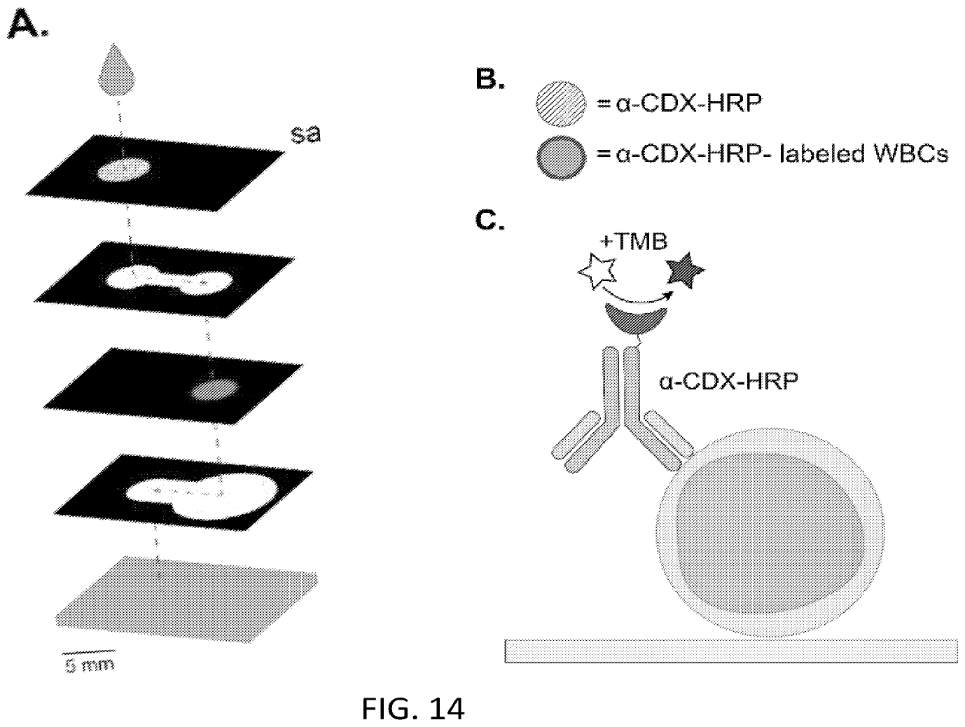

A fluidic device in which the WBCs are the “analyte” being detected via an antibody-reporter conjugate were created (FIG. 14). Briefly, the device comprised five layers: (i) a vertical transport layer, (ii) a lateral transport layer, (iii) a sample collection layer, (iv) a wash layer, and (v) a blotting layer (FIG. 14A). These layers were formed from a Clever coffee filter, a Clever coffee filter, a PES membrane having a maximum pore size of 0.8 μm, a Whatman 4 filter, and a ShamWow, respectively. When the sample, containing a suspension of WBCs, was added to the vertical transport layer, the WBCs interacted with an affinity reagent stored therein, a CD marker antibody conjugated to HRP (FIG. 14B). Next, the cell suspension wicked vertically to the lateral transport layer below, where the channel allowed the WBCs to mix with and bind the affinity reagent. Next, the cells wicked vertically to the subsequent sample collection layer below, which retains the WBCs due to their much larger diameters (7-13 μm for MAVER-1 and Jurkat D1.1). The remaining fluid wicked through the wash layer to the final, blotting layer. Upon completion of wicking, the fluidic device was peeled to expose the readout layer, which retained both labeled and unlabeled WBCs (FIG. 14B). Through the addition of the HRP substrate 3,3′,5,5′-tetramethylbenzidine (TMB), labeled WBCs were detected colorimetrically (FIG. 14C). The signal produced corresponded to the number of WBCs in the sample collection region, allowing for the enumeration of the desired WBC populations.

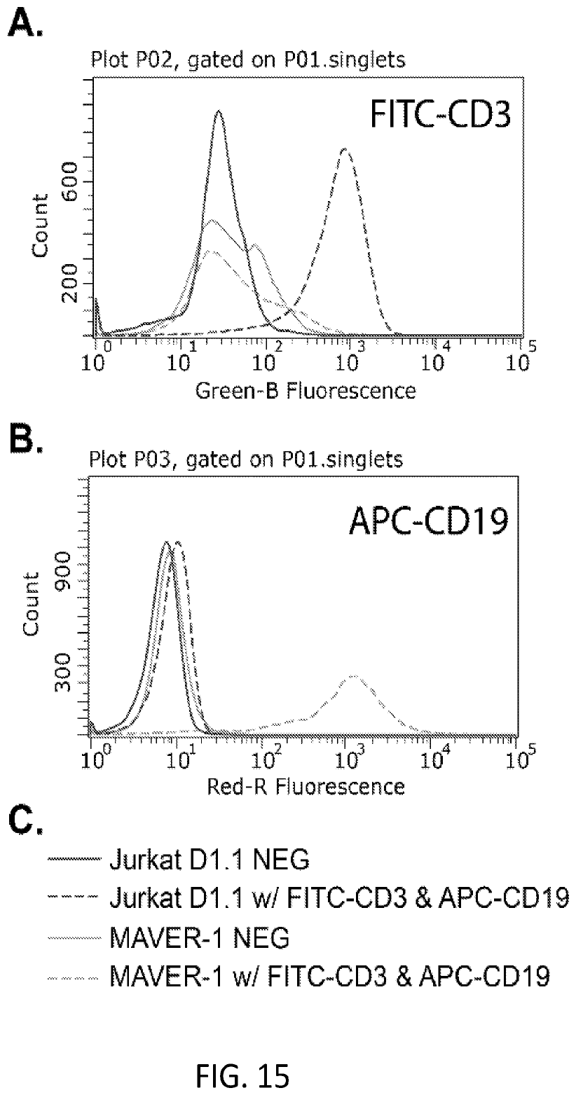

The vertical transport region was either treated with anti-CD3-HRP or anti-CD19-HRP: CD3 is a surface marker uniquely expressed on all T-lymphocytes and CD19 is a surface marker uniquely expressed on all B-lymphocytes. Therefore, if the device is treated with anti-CD3-HRP, it is believed that the readout zone will contain anti-CD3-HRP-labeled WBCs, which would permit the colorimetric detection of T-lymphocytes. Conversely, if the device is treated with anti-CD19-HRP, it is believed that the readout zone will contain anti-CD19-HRP-labeled WBCs, which would permit the colorimetric detection of B-lymphocytes. Two immortalized lymphocyte cell lines were selected to demonstrate the proof-of-concept testing of the fluidic devices due to their complementary immunophenotypes: Jurkat D1.1 (T-cell, CD3+/CD19−) and MAVER-1 (B-cell, CD3−/CD19+). Their surface marker signatures were confirmed by flow cytometry, as shown in FIG. 15. Additionally, these results indicated that the selected surface marker antibodies (anti-human CD3, clone SK7 and anti-human CD19, clone SJ25C1) adequately and specifically labeled each cell type.

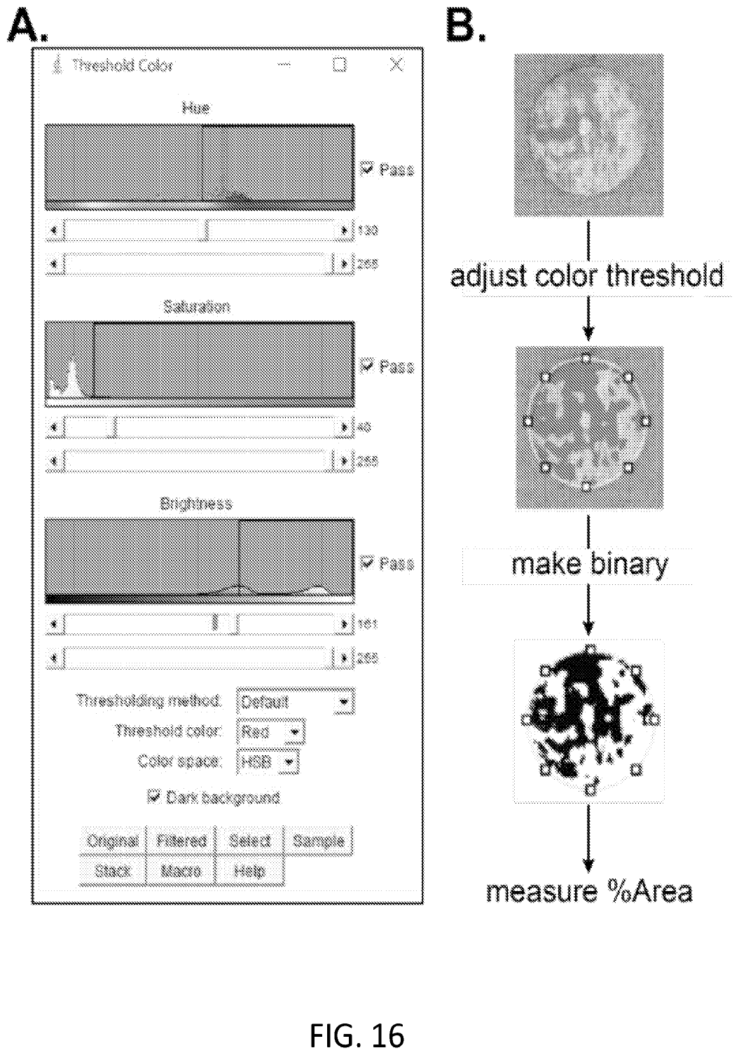

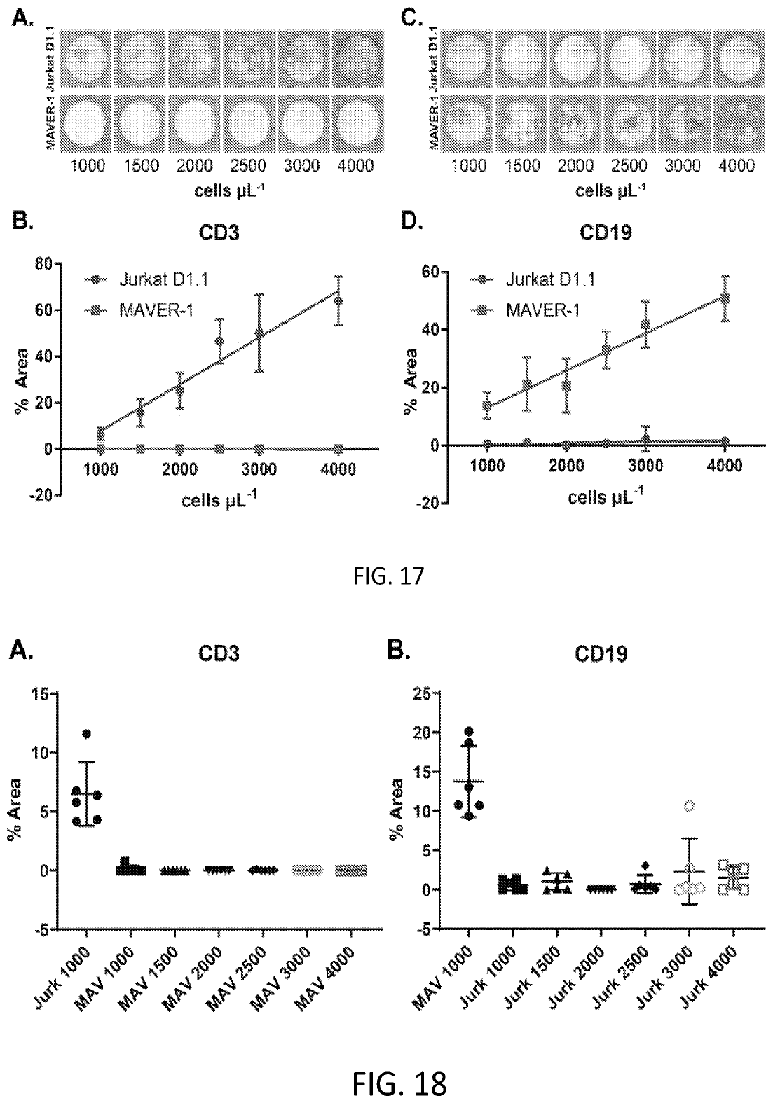

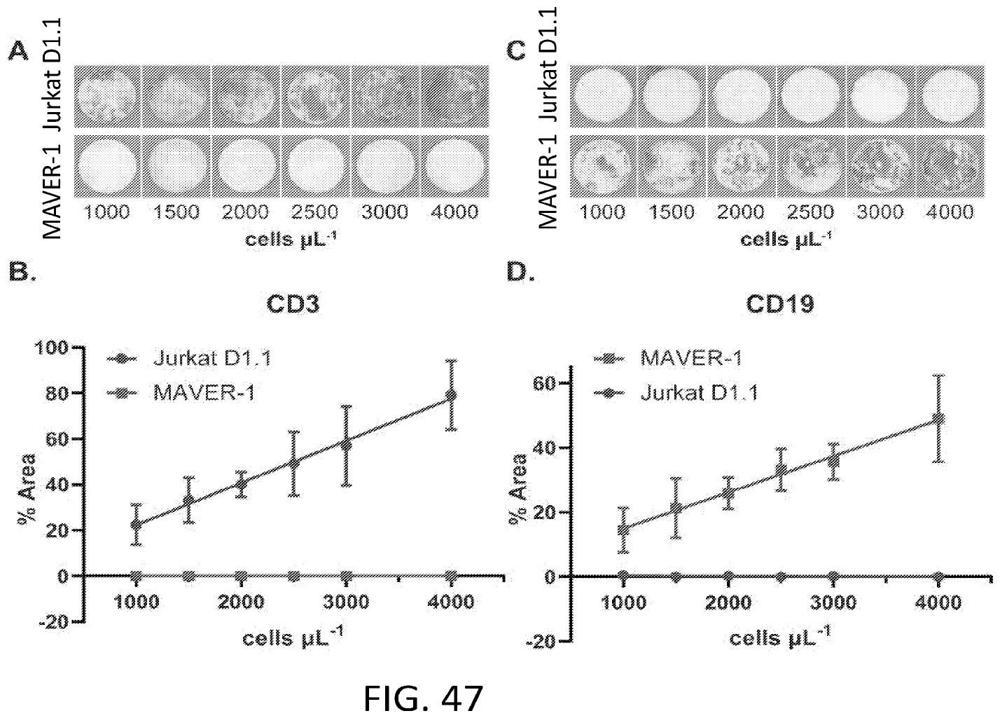

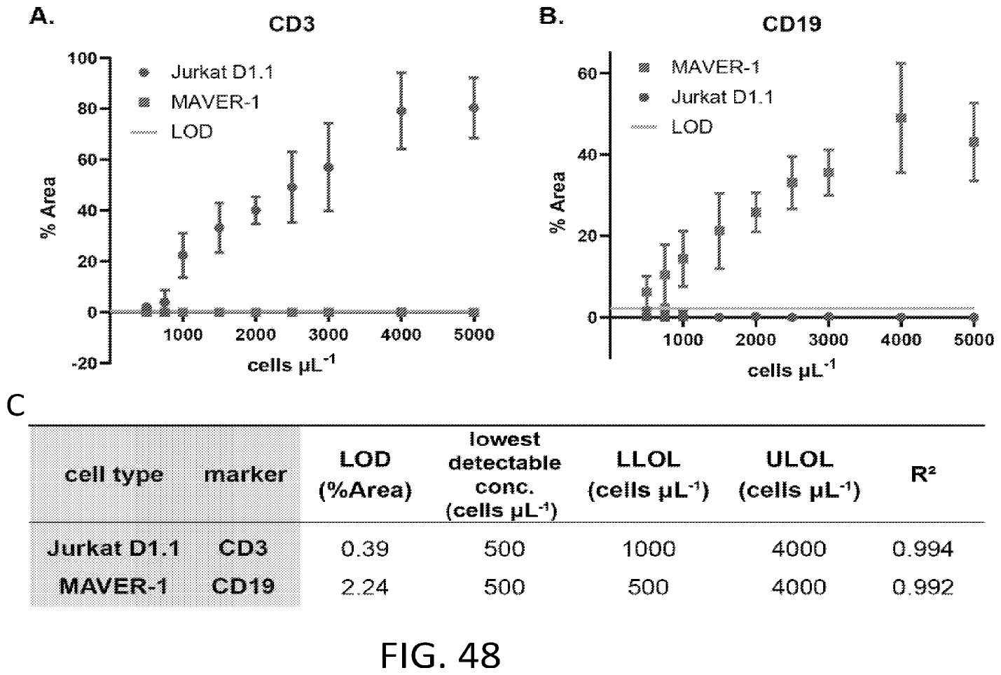

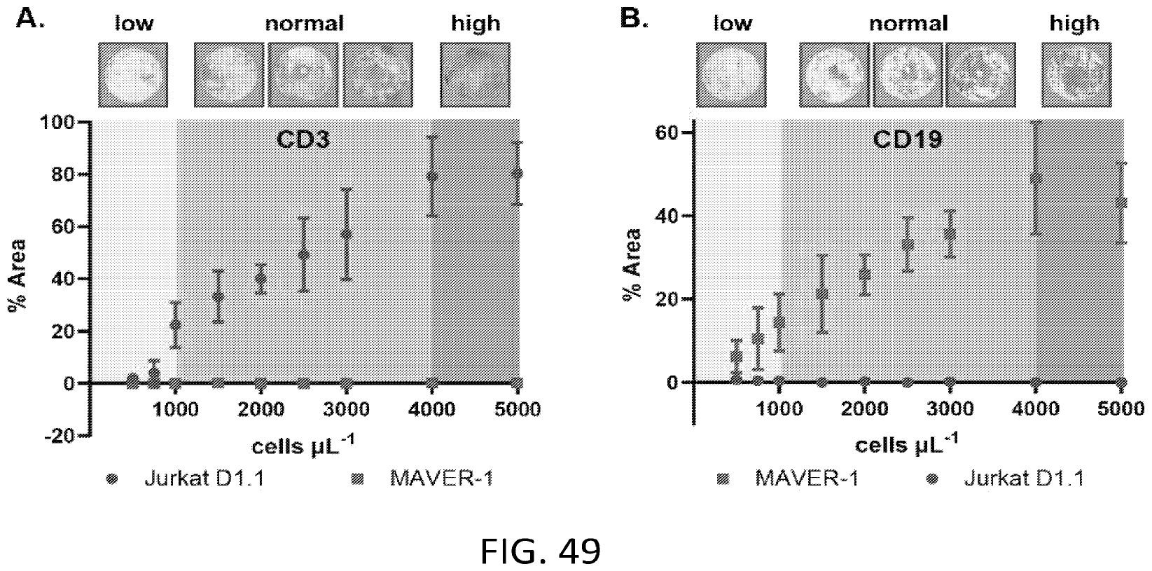

To demonstrate that the fluidic devices described herein could generate a specific, colorimetric signal for both T and B lymphocytes across the relevant physiological range, calibration curves were generated for each individual CD-marker using the appropriate positive- and negative-control cell cultures. Each cell type was assayed across the expected physiological range for total lymphocytes, 1000-4000 cells μL⁻¹. Briefly, to generate each calibration curve, aliquots of both Jurkat D1.1 and MAVER-1 cells were removed from their flasks and pelleted by centrifugation at 400×g for 8 min. The cells were then resuspended in PBS and their concentrations were determined using an automated hemocytometer (Countess II). Dilutions were then prepared at each target cell count. A total of 75 μL of cell suspension (in 3×25 μL aliquots) was added to fluidic devices treated with anti-CD3-HRP to enumerate T-cells and devices treated with anti-CD19-HRP treated to enumerate B-cells. Through the addition of TMB, the labeled cells produced a colorimetric signal which increased with cell concentration. The colorimetric signal was quantified by measuring the % Arca in ImageJ (FIG. 16). % Area was used rather than average color intensity because it was found to be more representative of the signal produced by the labeled cells. The final calibration curves are shown in FIG. 17. Further calibration curves performed on the same fluidic device with the same cell types are shown in FIG. 47. FIGS. 48 and 49 further show data from cell counts beyond the normal expected range, including both pathologically high and low counts.

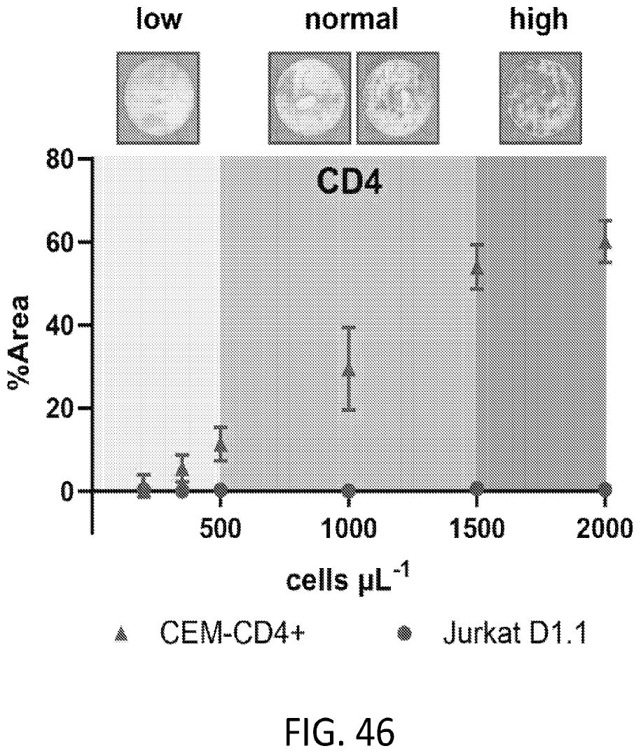

As evident in FIGS. 17 and 47, the labelling was specific for the target cell type (i.e., CD3+ Jurkat D1.1 and CD19+ MAVER-1 cells), as virtually no signal was observed from negative control cell lines for each assay across the entire physiological range. Additionally, the signals produced for each target cell type were linear across the physiological range, demonstrating that the fluidic devices produced a quantitative, colorimetric signal that corresponds to cell count. The CD3+ Jurkat D1.1 cells were detectable from fluids having cell concentrations of 1000-4000 cells μL⁻¹, with strong linear correlation between signal (quantified by % Area) and concentration (R²=0.955, FIG. 17B). Likewise, the CD19+ MAVER-1 cells were detectable from fluids having cell concentrations of 1000-4000 cells μL⁻¹ with an equally strong linear correlation (R²=0.959) between % Area and concentration (FIG. 17D). For both assays, the signal produced by the lowest tested concentration for the positive control (1000 cells μL⁻¹) was significantly different from the signals produced at all concentrations for the negative controls (1000-4000 cells μL⁻¹, FIG. 18), indicating that the lowest tested concentration was detectable above any background signal produced by negative controls. While the signals were quantified using image analysis, they were also detectable by eye across the entire physiological range for both target cell types, as shown in the representative scans in FIGS. 17A, 17C, 46A, and 46C. In this Example, a demonstration of a fluidic device capable of in situ labeling and detection of WBC subsets is presented. A material capable of transport of intact WBCs, a coffee filter, was also identified. The fluidic devices were employed enumerate two lymphocyte subsets, T- and B-cells, across the physiological range for total lymphocytes. The colorimetric signal produced was: (i) specific for the target cell population, (ii) linear across the intended physiological range when quantified by image analysis, and (iii) detectable by eye at all concentrations tested. It is also possible that the inclusion of both vertical and lateral flow channels within a fluidic device would allow for the potential incorporation of more complex sample processing, such as selective filtration of non-target cells. Additionally, multiplexing may also be supported. It is also believed that the fluidic devices described in this Example can be adapted for other target cell populations (e.g., WBCs or bacteria) using similar strategies of labeling cells via specific surface markers.

### Materials

The following cells were purchased: purified anti-human CD3 (clone SK7), purified anti-human CD19 (clone SJ25C1), FITC anti-human CD3 (clone SK7), and APC anti-human CD19 (clone SJ25C1) from BioLegend. Lightning-Link Horseradish Peroxidase Antibody Labeling Kit was purchased from Novus Biologicals. 3,3′,5,5′-tetramethylbenzidine (TMB Enhanced One Component HRP Membrane Substrate) was purchased from Surmodics, Inc. Bovine serum albumin (BSA) and phosphate-buffered saline (PBS, 10× stock diluted to 1× for use) were purchased from Fisher Bioreagents. Tween 20 and sucrose were purchased from Amresco. Blocker Casein in PBS (1% (w/v) casein) was purchased from Thermo Scientific. Whatman chromatography paper grade 4 (GE Healthcare Life Sciences), Clever coffee filters, and Shamwow were purchased from Amazon. 0.8 μm PES was purchased from Sterlitech. Flexmount Select DF051521 (permanent adhesive double-faced liner) and Flexmount Select DF021621 (removable/permanent adhesive-double faced liner) were purchased from FLEXcon (Spencer, MA). MAVER-1 (CRL-3008) and Jurkat D1.1 (CRL-10915) were purchased from ATCC. RPMI was purchased from Corning. Fetal bovine serum (FBS) and 1% penicillin-streptomycin were purchased from Gibco. DiI and DiO fluorescent stains were purchased from Invitrogen. Amicon Ultra 100K MW centrifugal filters were purchased from VWR. Suppliers for materials purchased for the material screen are listed in Table 1.

### Fabrication of the Fluidic Device

The wax-printed layers of the fluidic device (FIG. 14) were designed in Adobe Illustrator and patterned materials using hydrophobic wax printing (Xerox ColorQube 8580 printer). The wax was printed directly on the PES membrane for the sample collection layer and the Whatman 4 filter for the wash layer. A wax-transfer method was employed to print the wax onto the coffee filters. After printing or wax-transfer, the wax was melted to form hydrophobic barriers by using a Promo Heat press (PRESS-CS-15) at 280° F. For the sample collection and wash layers, the layers were heated with the Promo Heat press for 45 s to melt the wax through the full thickness of the layers. As the coffee filters are more porous, the wax flowed faster, so the vertical and lateral transport layers were heated for only 20 s to form the desired hydrophobic barriers. The adhesive layers were designed in Adobe Illustrator and cut using a BOSS LS1630 Laser cutter. A double-sided film with both removable and permanent adhesive was employed above the sample collection layer to facilitate peeling the fluidic device upon assay completion. All other layers were assembled using a double-sided permanent adhesive.

### Pore Size Characterization of Clever Coffee Filters

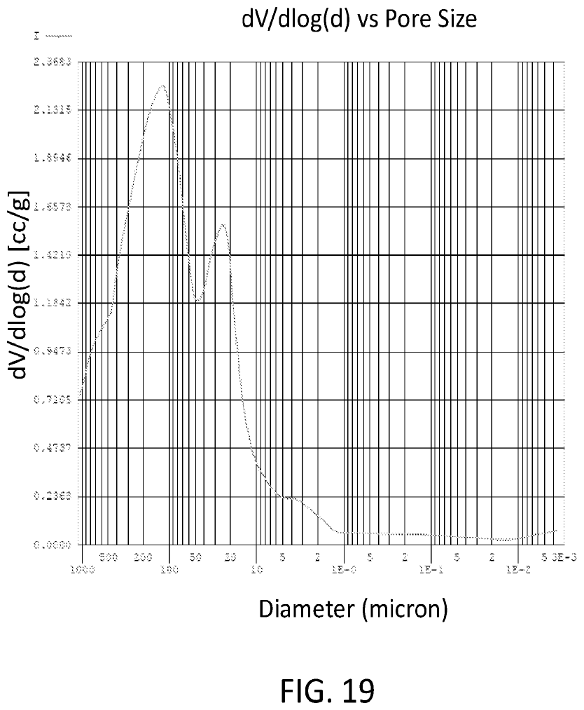

The pore sizes of the Clever coffee filters was characterized by mercury intrusion porosimetry (PoreMaster, Quantichrome). The pore size distribution showed two main populations of pore diameters: the first having a mode pore size of ˜25 μm and a second having a mode pore size ˜ 120 μm (FIG. 19). Both pore sizes were greater than the expected diameters of all white blood cells.

### Antibody Conjugation to Horse Radish Peroxidase (HRP)

To enable affinity-based colorimetric detection of the cells, the selected surface marker antibodies (i.e., anti-human CD3, anti-human CD19) were conjugated to horseradish peroxidase (HRP) using the Lightning-Link Horseradish Peroxidase Antibody Labeling Kit from Novus Biologicals following their established protocol. Briefly, purified antibody was first concentrated using 100K molecular weight cutoff centrifugation filters (to ˜4 mg mL⁻¹) to allow for optimal conjugation conditions. The appropriate volume of Lightning-Link modifier was added to the concentrated antibody, and this solution was then added directly to the provided vial of lyophilized material. The vial was allowed to incubate for ˜4 hours at room temperature in the dark. Finally, the appropriate volume of quencher reagent was added. The final volume of each conjugate was adjusted to 400 μL to dilute the conjugate to a workable concentration (˜1 mg mL⁻¹) for device treatment. The conjugates were stored in the dark at 4° C. until use.

### Fluidic Device Treatment

The vertical transport layer of the fluidic device were treated with 4 μL BSA (100 mg mL⁻¹ in PBS), which was allowed to dry at room temperature for 2 min and then at 65° C. for 5 min. The same region of the vertical transport layer (the vertical transport region) was then treated with 4 μL of 1:10 anti-CD3 HRP or 4 μL of 1:10 anti-CD19, which were diluted from ˜600 μg mL⁻¹ and ˜1000 μg mL⁻¹, respectively, using PBS. These volumes were determined to provide a strong signal for positive controls and a low background signal for the negative controls (data not shown). The conjugate was allowed to dry at room temperature for ˜20 min. To treat the sample collection layer, 10 μL of casein blocking buffer (1% (v/v) casein, 0.025 g mL⁻¹ sucrose, 0.1% (v/v) Tween 20, PBS) was added thereto. This layer was then allowed to dry for 2 min at room temperature and then for 12 min at 65° C. The remaining layers were not further treated.

### Fluidic Device Operation

To operate the fluidic devices, the cell suspension was added to the vertical transport region in 25 μL increments (3×25 μL). The addition of cells was followed by 2×25 μL of wash buffer, PBS. The addition of the wash buffer served two purposes: (i) to encourage cell transport to the sample collection layer and (ii) to remove unbound, HRP-conjugated antibody from the sample collection region to reduce the amount of background signal. Upon completion of flow, the fluidic device was peeled to expose the sample collection region. The sample collection layer was then removed from the fluidic device and affixed to a plastic tray so that the TMB droplet would incubate on the sample collection regions of the sample collection layer for the desired time (10 min). 15 μL of TMB was added to the sample collection region and allowed to react with labeled cells for 10 min. The TMB-HRP reaction was terminated by blotting all remaining TMB fluid through the sample collection region so that the membrane was dry. The fluidic devices were then scanned using an EPSON Perfection V600 Photo scanner.

### Cell Culture

MAVER-1 and Jurkat D1.1 cells were both cultured in suspension in RPMI 1640 medium supplemented with 10% fetal bovine serum (FBS) and 1% penicillin-streptomycin. The cell cultures were maintained at in a humid environment at 37° C. with 5% CO₂.

### Flow Cytometry

The immunophenotype signatures for each cell line were determined by flow cytometry (Luminex Corporation Guava easyCyte 12HT). Between 1-2× 10⁶ cells suspended in PBS were incubated with a cocktail of FITC-anti human CD3 and APC-anti human CD19 (5 μL of each) for 30 min at 4° C. on a tube rotator. A negative control of each cell type (i.e., no added antibody) was incubated alongside the positive control samples. The cells were washed twice with PBS by centrifugation at 400×g for 8 min. The flow cytometer was run using a low flow rate measuring 3×10⁵ events per well. Prior to gating, the proper compensation controls were applied using the easyCyte software. The cell populations were then gated for single cells and analyzed for green fluorescence (FIG. 15A) and red fluorescence (FIG. 15B) intensities.

### Image Analyses

To analyze the colorimetric signal produced by labeled cells on the sample collection regions, ImageJ was used to perform image analysis. The percentage of the total sample collection region that was occupied by cells (% Area) was quantified. To do this, ImageJ was used to adjust the color threshold using the settings shown in FIG. 16A to assign pixels to only the areas where a dark purple color is present (FIG. 16B), indicating the presence of HRP-labeled WBCs. Then, the image was converted to binary, and the % Area of pixels in the entire zone was measured (FIG. 16B).

### Statistical Analyses

To determine whether the signal produced by the lowest concentration tested (1000 cells μL⁻¹) for the positive control was detectable above the background signal produced by the negative controls at all concentrations (1000-4000 cells μL⁻¹), an ordinary one-way ANOVA with Tukey's multiple comparisons test (α=0.05) was performed. The results from this comparison showed that the mean signal produced by the CD3+ Jurkat D1.1 cells at 1000 cells μL⁻¹ was significantly different from the mean signals produced by the CD3-MAVER-1 cells at all concentrations tested (FIG. 18A). Additionally, the results showed that the mean signal produced by the CD19+ MAVER-1 cells at 1000 cells μL⁻¹ was significantly different from the mean signals produced by the CD19-Jurkat D1.1 cells at all concentrations tested (FIG. 18B).

### Fluorescent Mapping of Cells on the Sample Collection Layer

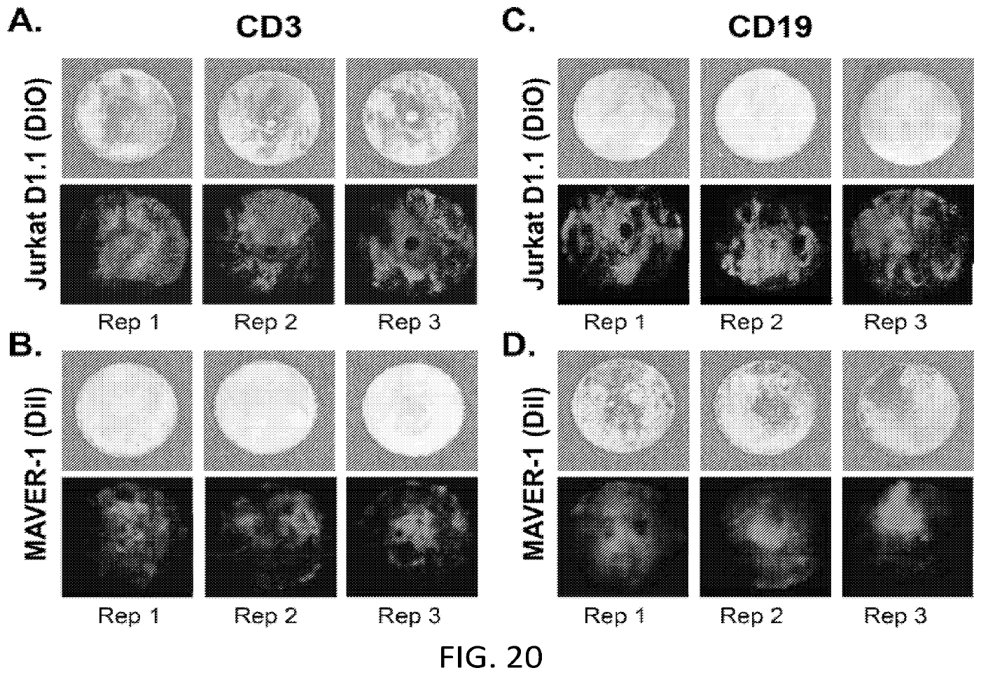

To confirm that the irregular colorimetric pattern produced by the captured cells on the sample collection layer was indeed due to how the cells are spreading, both cell populations were fluorescently labeled with a general membrane stain (DiO for Jurkat D1.1s and DiI for MAVER-1s) and added to devices treated with anti-CD3-HRP and anti-CD19-HRP. Then, TMB was added to the sample collection regions (as described in the device operation section above) and the sample collection regions were imaged the colorimetric signal on a scanner. Next, the sample collection regions were affixed face-down on a glass microscope slide and fluorescence images were acquired using a Leica DMi8 microscope equipped with a CoolLED pE-4000 light source and an Andor Revolution DSD2 confocal imaging system at 2.5× magnification. The results from this experiment are shown in FIG. 20. As evident in FIG. 20, the green-fluorescent signal produced from the DiO Jurkat D1.1s maps well with the colorimetric readout produced from the CD3-HRP labeled Jurkat D1.1s (FIG. 20A). The same result is seen for the DiI MAVER-1s, which produce a red-fluorescent signal that overlays with the colorimetric signal due to the CD19-HRP (FIG. 20D). Additionally, this experiment indicates that both assays are specific, as the CD19-DiO Jurkat D1.1s were present in the sample collection region of the fluidic devices treated with CD19-HRP, and no colorimetric signal was present (FIG. 20C). Conversely, the DiI MAVER-1s were clearly present on the capture zone of the devices treated with CD3-HRP, but they produced no colorimetric signal as they are CD3-negative (FIG. 20B).

### Example 2

This Example illustrates the ability of fluidic devices comprising polycarbonate track etched membranes (PCTEs) to capture monocytes.

Fluidic devices as shown in FIG. 21 were employed. These fluidic devices comprised a filter positioned between the top two layers (shown as gray circles). The filters were PCTE membranes comprising pores having both median and mode pore sizes of 10 microns, 12 microns, or 14 microns. All fluidic devices used a PES membrane comprising pores having a mode pore sizes of 5 microns for the sample collection layer (see layer labeled as “readout” in FIG. 21). The remaining layers in the fluidic devices were formed from the same materials described in Example 1.

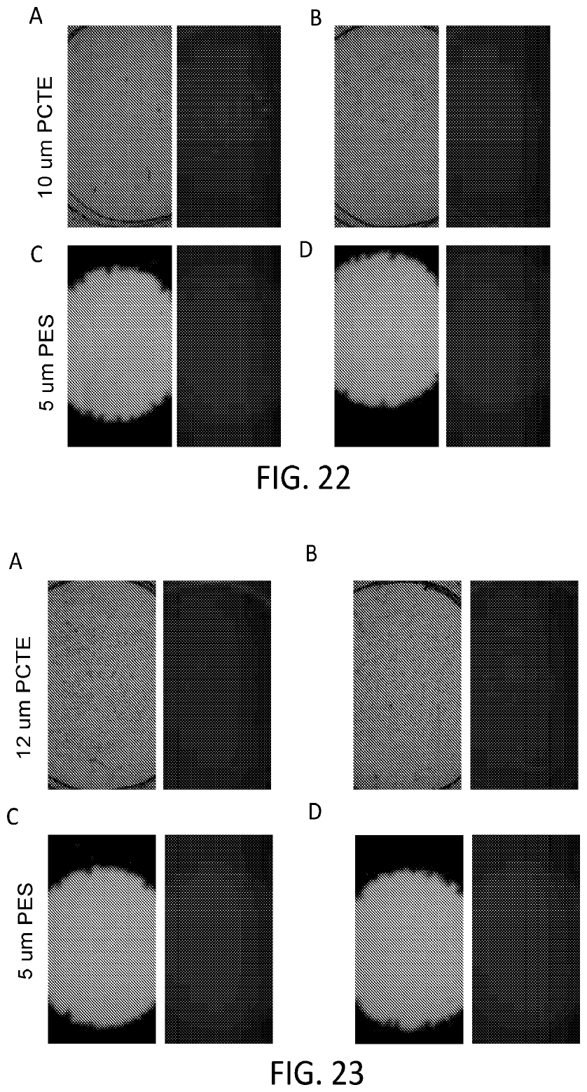

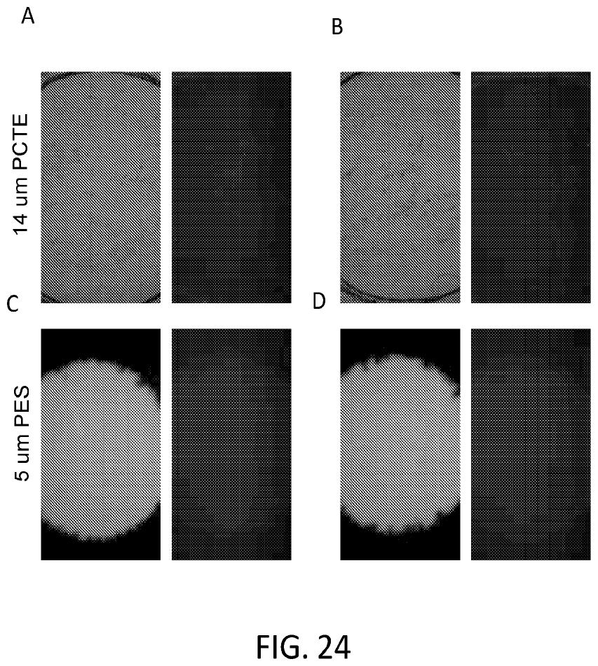

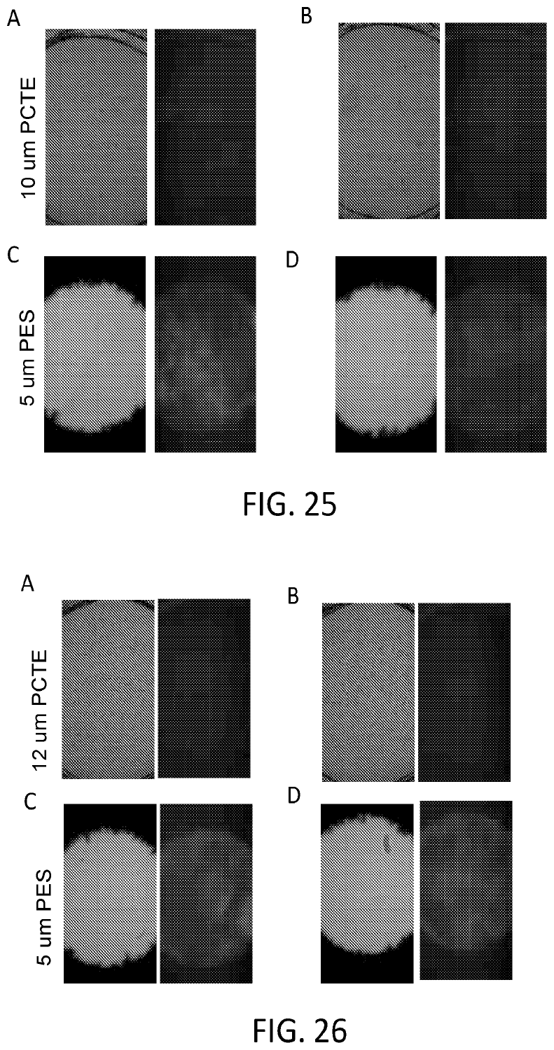

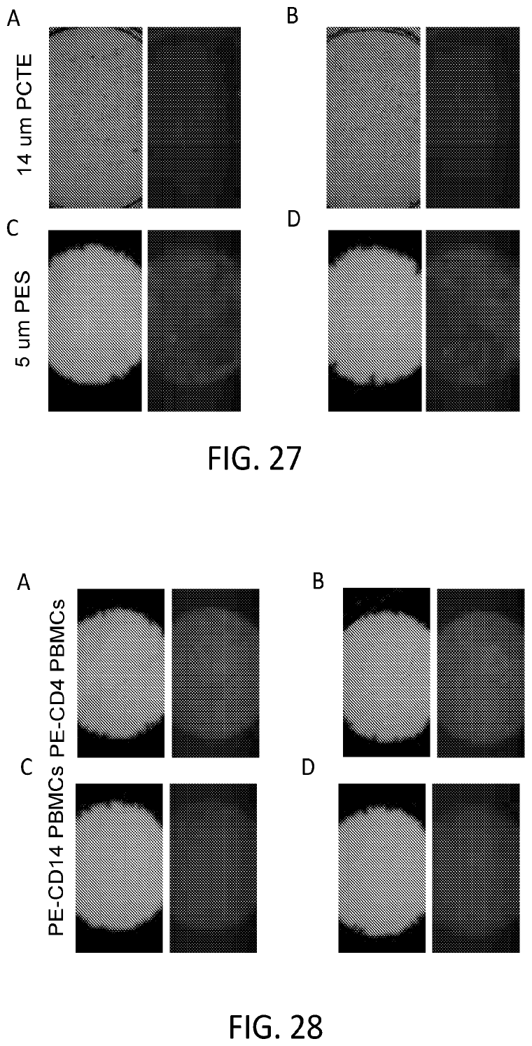

Monocytes were labeled in a sample of peripheral blood mononuclear cell (PBMCs) using PE-anti-CD14, suspended in 1X phosphate buffered saline (PBS), and then added to the above-described fluidic devices. Upon completion of the flow of the fluids comprising the monocytes through the fluidic devices, the filters and sample collection layers were removed and imaged via fluorescence microscopy. The resultant images indicated that monocytes were present on the PCTE membranes having 10 micron (FIGS. 22A-B), 12 micron (FIGS. 23A-B), and 14 micron (FIGS. 24A-B) pores and absent from the sample collection layer (see FIGS. 22C, 22D, 23C. 23D, 24C, and 24D). This suggests that the monocytes were captured by the filers and did not reach the sample collection layer.

### Example 3

This Example illustrates the ability of some fluidic devices comprising filters to both pass CD4+ T-cells through the filters and capture CD4+ monocytes on the filters.

CD4+ T-cells and CD4+ monocytes were labeled using PE-anti-CD4, suspended in 1×PBS, and added to the fluidic devices described in Example 2. Upon completion of flow, the PCTE membranes and the sample collection layers were removed and imaged via fluorescence microscopy. The resultant images indicated that CD4+ monocytes (and potentially some CD4+ T cells) were present on the PCTE membranes having 10 micron (FIGS. 22A-B), 12 micron (FIGS. 23A-B), and 14 micron (FIGS. 24A-B) median and mode pore sizes. The results also showed that CD4+ T-cells passed through the PCTE filters having 10 micron (FIGS. 22C-D), 12 micron (FIGS. 23C-D), and 14 micron (FIGS. 24C-D) pores to the sample collection layer.

### Example 4

This Example illustrates the ability of some fluidic devices lacking filters to pass PBMCs therethrough.

Suspensions of PMBCs including CD4+ T cells (25-60%), CD8+ T-cells (5-30%), B cells (5-10%), NK cells (10-30%), monocytes (5-10%) and dendritic cells (1-2%) were prepared in 1×PBS from whole blood. Separate aliquots of PBMCs were labeled with either PE-anti-CD4 or PE-anti-CD14 and added to devices having a structure similar to that described in Example 2 but lacking a filter.

Upon completion of the flow, the fluidic devices were peeled apart and the sample collection layers were imaged by fluorescent microscopy. PE-anti-CD4 labeled PBMCs (mainly CD4+ T-cells) were visible on the sample collection layers of devices to which they were added (FIGS. 28A-B). Similarly, PE-anti-CD14 PBMCs (monocytes) were visible on the sample collection layers of the devices to which they were added (FIGS. 28C-D).

Collectively this data shows that devices lacking filters do not impede cell flow through the device. FIGS. 28C-D show intense fluorescent staining on the sample collection region, suggesting that a high population of CD14+ cells have successfully traversed through the various layers of the device. Likewise, FIGS. 28A-B show intense fluorescent signal on the sample collection region, suggesting that a high population of CD4+ cells successfully traversed through the various layers of the device.

### Example 5

This Example describes performing multiplexing in a fluidic device.

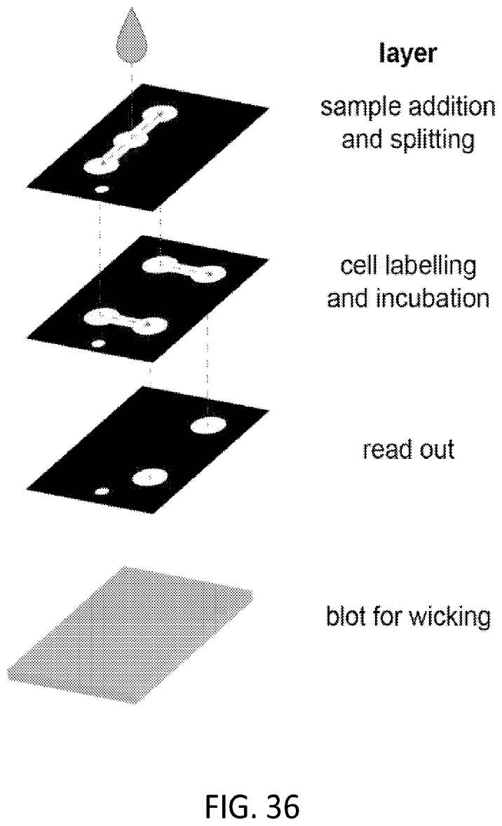

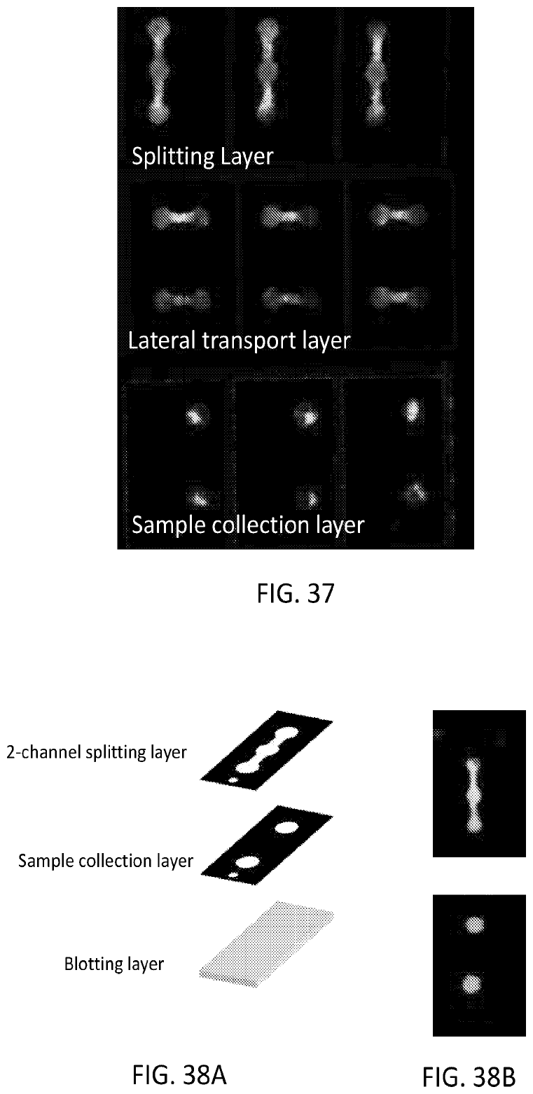

MAVER-1 B cells were pre-stained with DiI (a cell membrane stain) and suspended in 1×PBS to form a fluid sample. The fluid sample was then added to the multiplexed device shown in FIG. 36. Following addition of the fluid sample to the central sample addition region of a 2-channel splitting layer, the fluid sample was wicked in opposite directions along a first channel and a second channel towards a first split sample region and a second split sample region, thus splitting the sample into two distinct flow paths. The fluid in the first split sample region flowed laterally downward through the first split sample region to a first sample reception region in a lateral transport layer placed under the 2-channel splitting layer. Likewise, the fluid in the second split sample flowed laterally downward through the second split sample region to a second sample reception region in the lateral transport layer. The fluid samples in the first and second sample reception regions then flowed laterally through channels in the lateral transport layer toward the third and fourth sample reception regions. The fluid samples then flowed vertically through the third and fourth sample reception regions, and onto sample collection regions on a sample collection layer placed under the lateral transport layer. After the fluid sample was finished flowing through the fluidic device, the layers were peeled apart and imaged using fluorescent imager. As shown in the FIG. 37, fluorescently-labeled MAVER-1 cells were present in both channels in the 2-channel splitting layer, along the channels in the lateral transport layer, and within all sample collection regions of the sample collection layer, suggesting that fluid samples comprising cells can be split into multiple flow paths for multiple analyses. Experiments were run in triplicate.

It should also be noted that similar processes can be performed in fluidic devices further comprising other components and/or layers, such as the fluidic device shown in FIG. 29, which is similar to the fluidic device shown in FIG. 36 but further comprises filters and a wash layer.

### Example 6

This Example describes performing multiplexing in a fluidic devices comprising splitting layers having various designs.

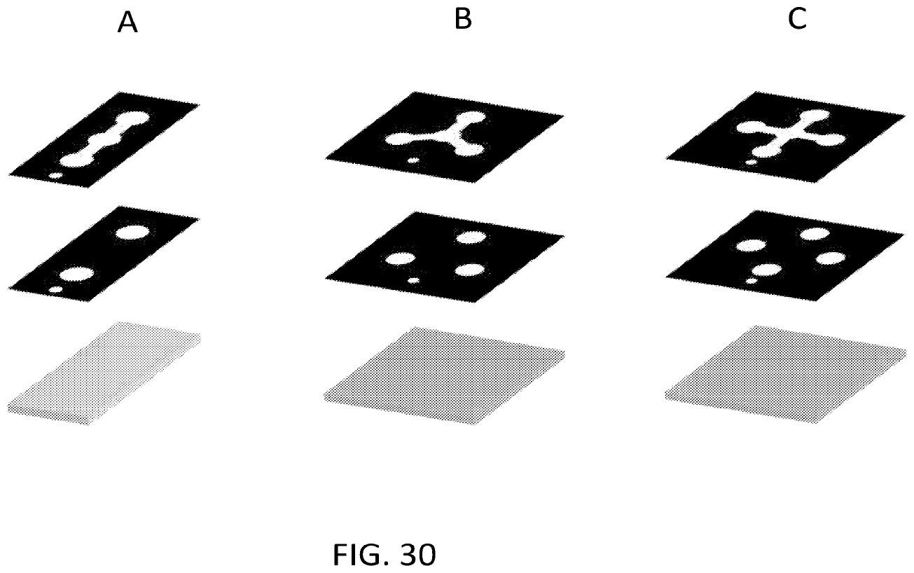

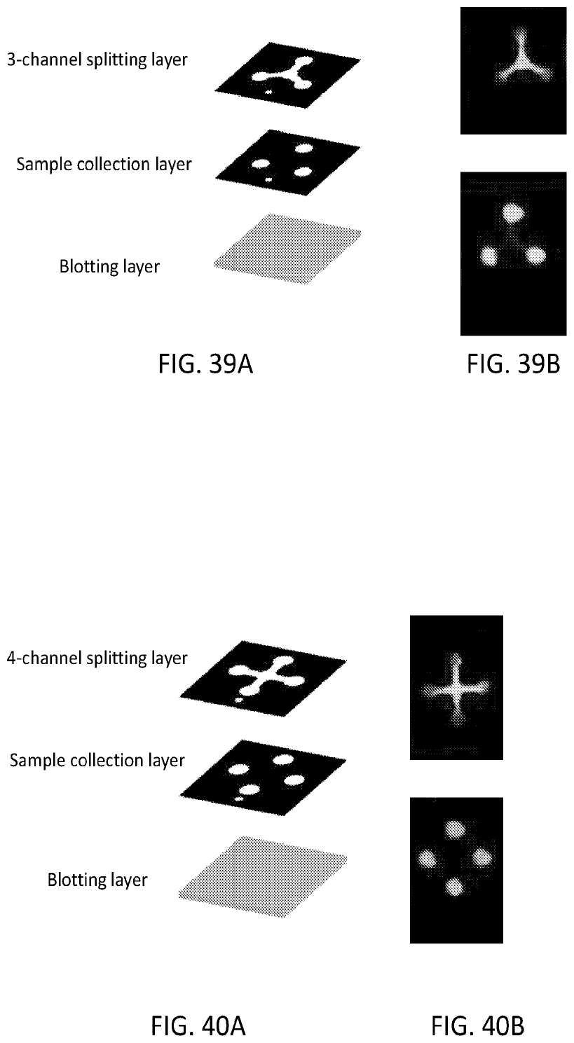

Fluid samples were prepared as described in Example 5 and added to devices comprising either a 2-channel splitting layer (FIG. 38A), a 3-channel splitting layer (FIG. 39A), or a 4-channel splitting layer (FIG. 40A). These fluidic devices are also shown in FIG. 30. After flow was complete, the layers were peeled apart and imaged using a fluorescent imager. As shown in FIGS. 38-40, the fluid sample was split into 2 (FIG. 38B), 3 (FIG. 39B), and 4 channels (FIG. 40B), and passed vertically through the splitting layer onto a sample collection layer disposed beneath it. This example confirmed that 2-plexed, 3-plexed, and/or 4-plexed readouts are possible from a single aliquot of the starting sample.

It should also be noted that similar processes can be performed in fluidic devices further comprising other layers, such as filters and/or wash layers.

### Example 7

This Example describes a fluidic device comprising a vertical transport layer treated with a protein A-cellulose binding domain conjugate (ProA-CBD).

Fluidic devices as shown in FIG. 31 were employed. These fluidic devices comprised a vertical transport layer with two vertical transport regions positioned on top of a lateral transport layer with two channels. The vertical transport region comprised a protein A-cellulose binding domain conjugate capable of binding to the Fc portion of an antibody. All fluidic devices used a PES membrane comprising pores having median and mode pore sizes of 5 microns for the sample collection layer. The remaining layers in the fluidic devices (i.e., the wash layer and the blotting layer) were formed from the same materials described in Example 1.

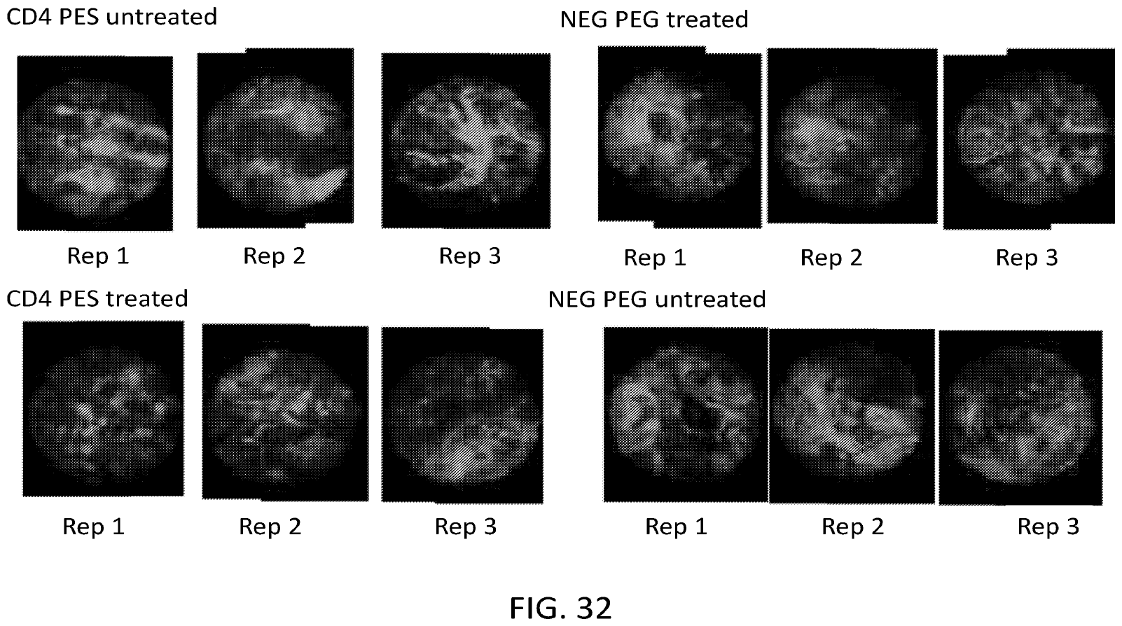

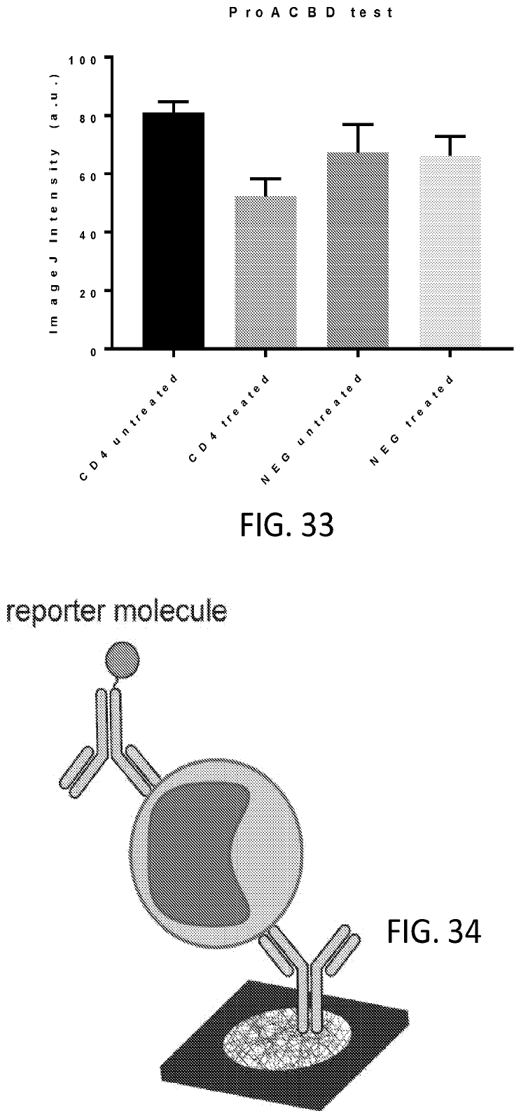

CEM-CD4+ cells, which are a T lymphoblast cell line, were stained with DiI, a red fluorescent general membrane marker, and incubated with purified anti-human CD4 antibodies. Additionally, an aliquot of DiI stained CEM-CD4+ cells were not labeled with the anti-human CD4 antibody were used as a negative control. The anti-human CD4 antibody-labeled cells were then added to devices having the structure shown in FIG. 31. Upon completion of the flow of the fluids comprising the CEM-CD4+ cells through the fluidic devices, the sample collection layers were removed and imaged via fluorescence microscopy. The results showed a loss in fluorescent signal when anti-CD4-labeled CEMs were added to devices treated with ProA-CBD (FIG. 32B; FIG. 33, see CD4 treated), in the vertical transport region, compared to untreated devices (FIG. 32A; FIG. 33, see CD4 untreated). The results also showed no loss in fluorescent signal when non-labeled CEM cells were added to devices, regardless of whether or not the device had been treated with ProA-CBD (FIGS. 32C-D; FIG. 33, see NEG untreated and NEG treated), suggesting that the presence of ProA-CBD does not bind cells non-specifically. Collectively, these results suggest that devices comprising ProA-CBD can be used to deplete a target cell population within a fluid sample.

While not explicitly shown in this example, it is also contemplated that the ProA-CBD could be used to achieve specific capture of a desired cell type, as opposed to depleting a cell type from a fluid sample, as described herein. In such devices, the vertical transport layer would be removed from the device and stained for quantification.

### Example 8

This Example describes a fluidic device comprising a vertical transport layer chemically treated to allow for specific capture of one or more desired cell types from a liquid solution.

Fluidic devices as shown in FIG. 14 were employed. These fluidic devices comprised a vertical transport layer made of polyester positioned on top of a lateral transport layer. The polyester vertical transport layer was chemically modified with anti-CD4 or anti-CD20 antibodies. All fluidic devices employed a PES membrane having mean and mode pore sizes of 5 microns for the sample collection layer. The remaining layers in the fluidic devices (i.e., the wash layer and the blotting layer) were formed from the same materials described in Example 1.

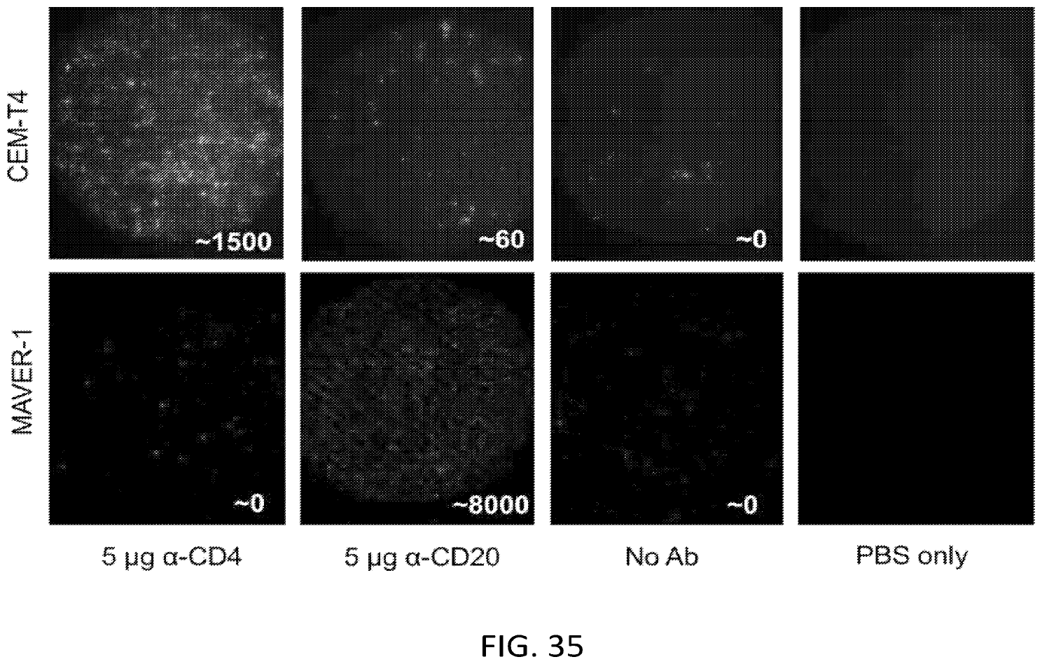

CEM cells (CD4+/CD20−) were stained with DiO (FIG. 35, green fluorescent signal) and MAVER-1 B cells (CD4−/CD20+) were stained with DiI (FIG. 35, red fluorescent signal). The CEM labeled cells were then added to devices whose vertical transport layer was chemically modified with anti-CD4, whereas the MAVER01 labeled cells were added to devices modified with anti-CD20 antibodies. Upon completion of the flow of the fluids comprising the CEM (CD4+/CD20−) or MAVER-1 (CD4−/CD20+) cells through the fluidic devices, vertical transport layers were removed and imaged via fluorescence microscopy (FIG. 35). The results showed that CEM (CD4+/CD20−) cells become trapped within vertical transport layers modified with CD4 (FIG. 35A), but not CD20 (FIG. 35B); likewise MAVER-1 cells become trapped within vertical transport layers modified with CD20 (FIG. 35C), but not CD4 (FIG. 35D). Control experiments confirmed that both cell types freely passed through unmodified polyester membranes (FIGS. 35E-F) and that the carrier fluid (e.g. phosphate-buffered saline) is non-fluorescent (FIGS. 35G-H).

### Example 9

This Example compares the pore size distribution for three different porous, absorbent layers, assesses these pore size distributions in comparison to MAVER-1 cell size, and describes how the pore size distribution may affect MAVER-1 cell transport therethrough.

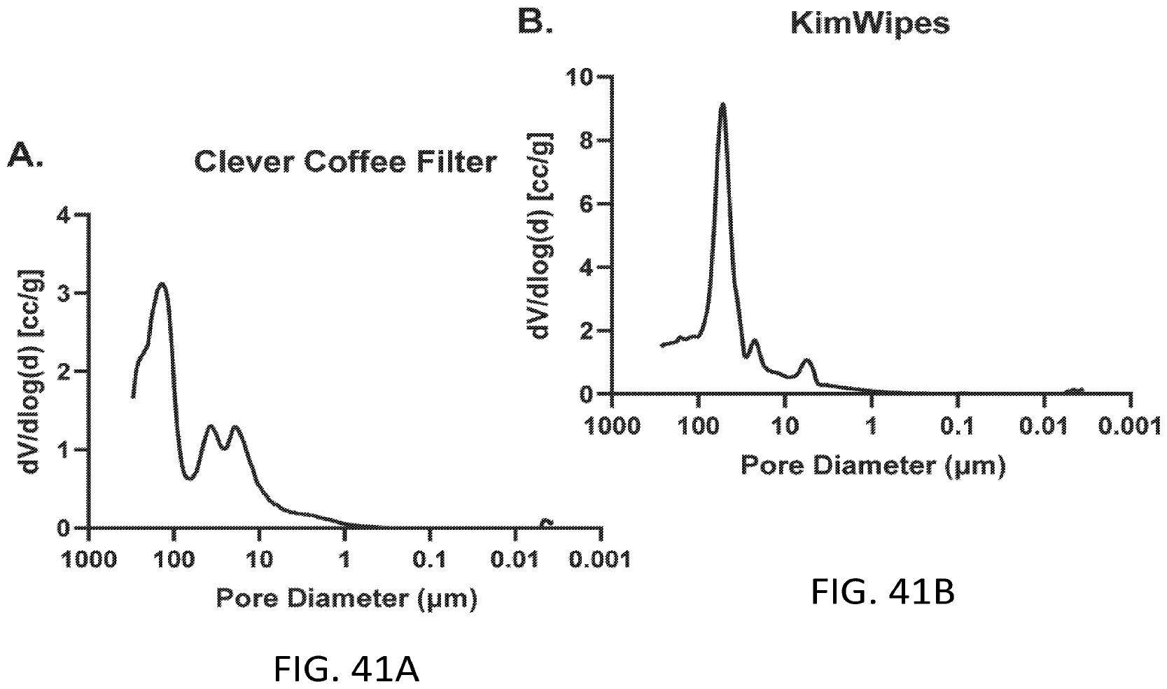

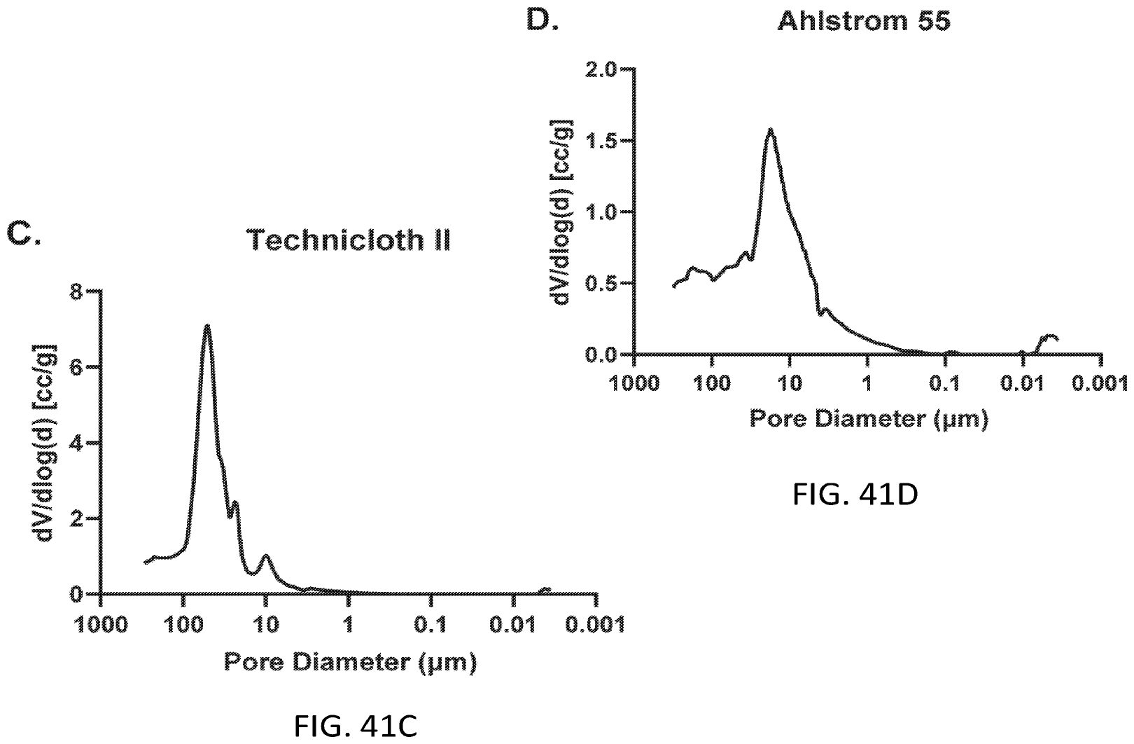

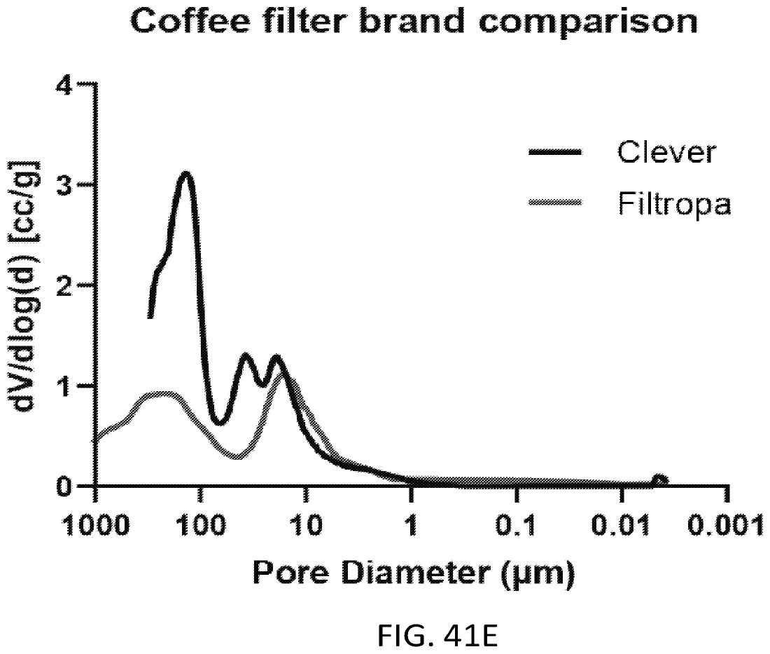

Mercury intrusion porosimetry measurements were performed on a Clever coffee filter, a Kimwipe, a Technicloth II TX1109 synthetic wiper, an Ahlstrom 55 filter paper, and a Filtropa coffee filter. From these measurements, the pore size distribution, volume median pore diameter, peak pore size, and percent porosity of these materials. The results are shown in FIGS. 41A-41E and Tables 2 and 3.

TABLE 2

| | Volume median | Peak pore sizes | % |
|---|---|---|---|
| Material | pore diameter (μm)* | (μm)^{†} | Porosity* |
| Clever coffee filters | 108.6 | 18.5, 36.5, 135 | 69 |
| KimWipes | 52.8 | 5.5, 22.2, 52.8 | 87 |
| Technicloth II | 48.5 | 9.8, 23.5, 51.3 | 85 |
| Ahlstrom 55 | 18.8 | 3.5, 5, 6.4, 17.3, | 76 |
| | | 36.4, 57.5, 180 | |

*measured by mercury intrusion porosimetry
^{†}calculated by peak fitting

TABLE 3

| Material | % pores <10 μm | % pores <20 μm | % pores <30 μm |
|---|---|---|---|
| Clever coffee | 0.43 | 2.12 | 3.99 |
| filters | | | |
| KimWipes | 0.79 | 2.11 | 4.40 |
| Technicloth II | 0.68 | 2.16 | 6.61 |
| Ahlstrom 55 | 2.76 | 10.11 | 15.47 |

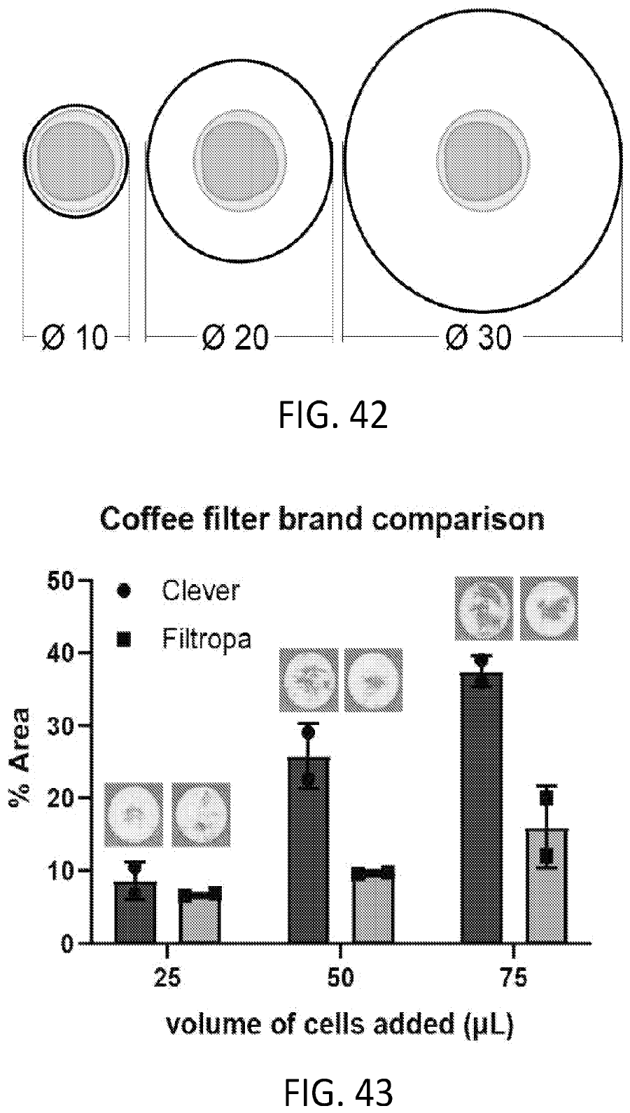

FIG. 42 compares the size of MAVER-1 cells (shown in gray) to 10 micron, 20 micron and 30 micron pores. These cutoffs were selected as they roughly indicate 1X, 2X and 3X the diameter of the MAVER-1 cells that were used to perform the material screen. As shown in Table 3, the materials with lower percentages of pores below each cutoff allowed for better white blood cell (WBC) transport. Specifically, the Clever coffee filters and KimWipes had the lowest percentages (<1% below 10 μm, ˜2% below 20 μm, and <5% below 30 μm), and correspondingly also demonstrated good MAVER-1 transport as shown in FIG. 13. The Technicloth II synthetic wipers had similar percentages when compared to the Clever coffee filters and KimWipes at the 10 and 20 μm cutoffs but deviated slightly (>5%) at the 30 μm cutoff, which may suggest that clogging can occur at pore sizes as large as 30 μm in diameter, as the cell transport in Technicloth II wipers was reduced in comparison to Clever coffee filters and KimWipes (FIG. 13). Ahlstrom 55 demonstrated higher percentages of pores at each cutoff (>2% below 10 μm, ˜10% below 20 μm, and ˜15% below 30 μm), which may suggest a reason for poor WBC transport through Ahlstrom 55.

A fluidic device having the design described in Example 1 and shown in FIG. 14 was fabricated except that it employed a Filtropa coffee filter lacking an affinity reagent instead of a treated Clever coffee filter as the vertical transport layer. Cell transport through this fluidic device was assessed in the same manner described in Example 1, except that it was performed on fluid samples comprising pre-labeled CEM-CD4+ cells with anti-CD4-HRP at a concentration of 2000 cells μL⁻¹ in volumes of 25, 50, and 75 μL. The results of this test are shown in FIG. 43.

Although the two coffee filters demonstrate similar signal at a fluid sample volume of 25 μL, the Clever brand coffee filter outperforms the Filtropa brand coffee filter at larger fluid sample volumes. This may be explained by the different pore size distribution of the Filtropa brand coffee filters, which exhibited a volume median pore diameter of 81.4 μm, peak pore sizes of 16.4 μm and 209.7 μm, an amount of pores below 10 μm of 0.53%, an amount of pores below 20 μm of 2.07%, and an amount of pores below 30 μm of 3.15%. In comparison with the Clever brand coffee filter, (i) the volume median pore diameter for Filtropa is smaller and (ii) one of the peak pore populations is at a pore size of 16.4 μm, which is smaller than the smallest peak pore size of Clever brand.

### Example 10

This Example describes the use of a fluidic device to capture and detect CEM-CD4+ T cells.

A fluidic device was designed to detect CD4+ T cells. This fluidic device was the same as the fluidic device described in Example 1 and shown in FIG. 14A except that the vertical transport layer was functionalized with anti-CD4-HRP instead of the affinity reagent described in Example 1. CD4+T lymphocyte counts normally range between 500 and 1500 cells μL⁻¹ in a healthy adult. However, low CD4+ T cell counts can be used to identify persons with advanced HIV disease/AIDS and to inform clinical management.

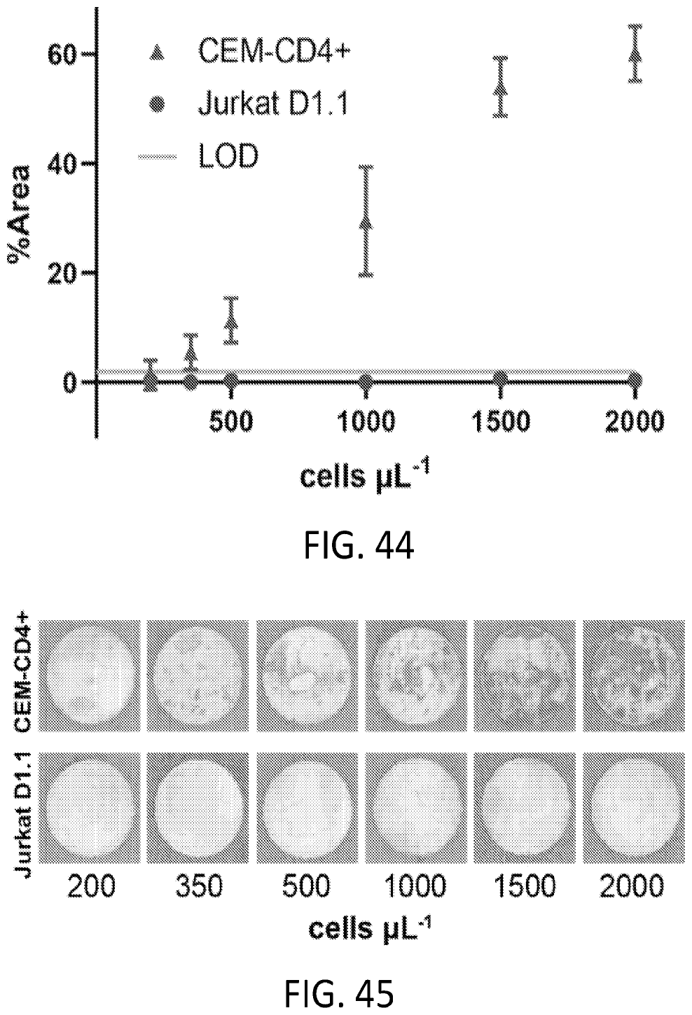

The fluidic device designed to detect CD4+ T cells was then exposed to fluids comprising CD4+ (CEM-CD4+) and CD4-T cell lines (Jurkat D1.1) in varying amounts. Calibration curves were then built in a similar manner as described above in Example 1 but that included concentrations spanning the physiological range for this subset of T cells, 200-2000 cells μL⁻¹. The full CD4 calibration curves are shown in FIG. 44 and associated representative scans are shown in FIG. 45. This CD4 assay is also highly specific for the target cell type, CEM-CD4+ cells. The CD4 assay is linear from 200-1500 cells μL⁻¹ (R²=0.993), and the lowest detectable concentration by image analysis is 350 cells μL⁻¹. The signal at 200 cells μL⁻¹ falls below the LOD and is undetectable by image analysis, and the signal produced at 2000 cells μL⁻¹ saturates the calibration curve. However, these features can be beneficial for providing semi-quantitative readouts. For instance, FIG. 46 demonstrates how this assay may be employed to provide a semi-quantitative readout for HIV disease/AIDS.

### Example 11

This Example describes the fluorescent imaging of the sample collection layer of an exemplary fluidic device after the passage therethrough of a fluid sample comprising fluorescently labeled MAVER-1 cells.

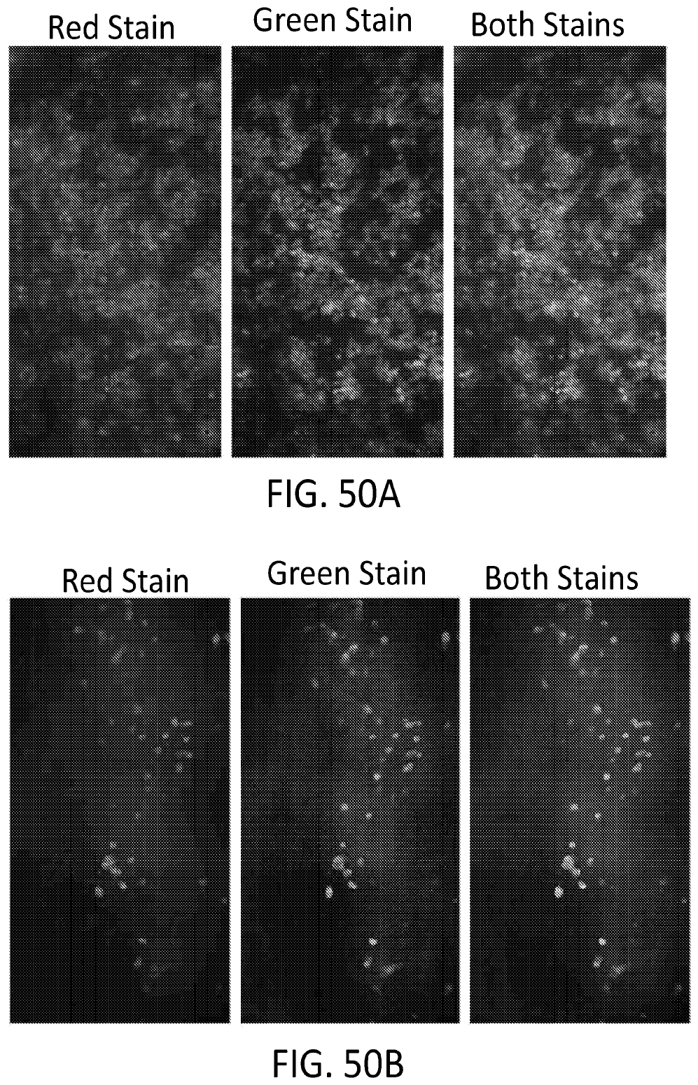

MAVER-1 cells were fluorescently labeled with a general membrane stain (DiI, red) and a nuclear stain (SYTO 9, green), suspended in 1×PBS to form a fluid sample as described in Example 1, and then added to fluidic devices having the design described in Example 1 and shown in FIG. 14A. After flow was complete, the sample collection layers were affixed face-down on a glass microscope slide and subjected to fluorescence imaging using a Leica DMi8 microscope equipped with a CoolLED pE-4000 light source and an Andor Revolution DSD2 confocal imaging system at 20× magnification in confocal mode. For one set of devices, the sample collection layer was affixed face-down to the glass microscope slide once completely dry (FIG. 50A). For the other set of devices, the sample collection layer was affixed face-down to the glass microscope slide while still wet, with the hypothesis that intact cells could sediment from the sample collection layer onto the microscope slide to facilitate imaging (FIG. 50B). As shown in FIGS. 50A and 50B, the red and green fluorescence signals show good colocalization, indicating that the signals from the fluorescent membranes (red) and fluorescent nuclei (green) overlap well and suggesting that the cells are still intact. Autofluorescence and scattering from the dry sample collection layer makes individual cells difficult to resolve. However, cells recovered from the wet sample collection layer onto the glass slide are apparent, and more clearly confirm that the white blood cells stay intact when they reach the sample collection layer after being transported throughout the entire fluidic device.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A fluidic device, comprising:
a first layer comprising a porous, absorbent material having a median pore size of greater than or equal to 15 microns and a mode pore size of greater than or equal to 15 microns, wherein the first layer comprises a channel, a first sample reception region, and a second sample reception region, and wherein the channel places the first sample reception region in fluidic communication with the second sample reception region; and
a second layer wherein the second layer comprises a vertical transport region in fluidic communication with the first sample reception region and/or a sample collection region in fluidic communication with the second sample reception region.

2. A fluidic device, comprising:
a first layer comprising a first porous, absorbent material, wherein the first layer comprises a channel, a first sample reception region, and a second sample reception region, and wherein the channel places the first sample reception region in fluidic communication with the second sample reception region; and
a second layer comprising a second porous, absorbent material having a median pore size of greater than or equal to 15 microns and a mode pore size of greater than or equal to 15 microns, wherein the second layer comprises a vertical transport region in fluidic communication with the first sample reception region.

3. A method, comprising:
laterally transporting a fluid comprising a plurality of cells through a channel, wherein:
the channel is positioned in a first layer comprising a porous, absorbent material having a median pore size of greater than or equal to 15 microns and a mode pore size of greater than or equal to 15 microns,
the first layer further comprises a first sample reception region and a second sample reception region,
the channel places the first sample reception region in fluidic communication with the second sample reception region,
the cells are transported from the first sample reception region to the second sample reception region through the channel,
the first layer is positioned in a fluidic device further comprising a second layer, and
the second layer comprises a vertical transport region in fluidic communication with the first sample reception region and/or a sample collection region in fluidic communication with the second sample reception region.

4-7. (canceled)

8. The method of claim 3, wherein the fluid comprises cells.

9. (canceled)

10. The fluidic device of claim 1, wherein the second layer comprises the vertical transport region.

11. (canceled)

12. The fluidic device of claim 2, wherein the pores in the first porous, absorbent material are interconnected and the second porous, absorbent material comprises laterally isolated pores.

13-22. (canceled)

23. The fluidic device of claim 1, wherein the second layer comprises the sample collection region.

24. The fluidic device of claim 1, wherein the first layer is disposed on the second layer.

25. The fluidic device of claim 1, wherein the second layer comprises a polyethersulfone filter, a leukosorb membrane, or a nitrocellulose membrane.

26-27. (canceled)

28. The fluidic device of claim 1, wherein the second layer comprises a porous material having a median pore size of less than or equal to 1 micron.

29. The fluidic device of claim 1, wherein the second layer comprises the vertical transport region and the device further comprises a third layer comprising the sample collection region.

30. The fluidic device of claim 29, wherein the second layer and the third layer are positioned on opposite sides of the first layer.

31. The fluidic device of claim 29, further comprising a fourth layer, wherein the third layer is disposed on the fourth layer, wherein the fourth layer comprises a wash region, and wherein the wash region is in fluidic communication with the sample collection region of the second layer.

32-44. (canceled)

45. The fluidic device of claim 1, further comprising a filter.

46-50. (canceled)

51. The fluidic device of claim 45, wherein the filter comprises laterally isolated pores.

52. The fluidic device of claim 1, further comprising a splitting layer—wherein the splitting layer is disposed on the first layer, wherein the splitting layer comprises a 2-channel splitting layer, a 3-channel splitting layer, or a 4-channel splitting layer.

53-55. (canceled)

56. The fluidic device of claim 52, wherein the splitting layer comprises a central sample addition region.

57-58. (canceled)

59. The fluidic device of claim 1, wherein the vertical transport region comprises an affinity agent.

60. The fluidic device of claim 59, wherein the affinity agent is conjugated to a chromogen or a fluorophore.

61-79. (canceled)

80. The fluidic device of claim 1, wherein the sample collection region comprises a myeloperoxidase-specific substrate and/or a leukocyte esterase-specific substrate.

81-106. (canceled)
